(19) United States
(12) Patent Application Publication
(10) Pub. No.: US 2011/0308592 A1
(43) Pub. Date: Dec. 22, 2011

(54) USE OF SUBSTITUTED PERYLENES IN ORGANIC SOLAR CELLS

(75) Inventors: Martin KÖNEMANN, Mannheim (DE); Jae Hyung HWANG, Viernheim (DE); Gabriele MATTERN, Schifferstadt (DE); Regina HÖH, Waldsee (DE); Christian DÖRR, Niederkirchen (DE)

(73) Assignee: BASF SE, Ludwigshafen (DE)

(21) Appl. No.: 13/163,286

(22) Filed: Jun. 17, 2011

Related U.S. Application Data
(60) Provisional application No. 61/356,055, filed on Jun. 18, 2010.

(30) Foreign Application Priority Data
May 5, 2011 (EP) 11164893.7

(51) Int. Cl. H01L 51/46; H01L 31/06; C07D 487/04; C07D 221/18; C07C 13/62; C07D 209/56

(52) U.S. Cl. 136/255; 585/27; 548/417; 546/26; 546/28; 136/263

(57) ABSTRACT

The present invention relates to an organic solar cell with a photoactive region which comprises at least one organic donor material in contact with at least one organic acceptor material, wherein the donor material and the acceptor material form a donor-acceptor heterojunction and wherein the photoactive region comprises at least one substituted perylene.

### BACKGROUND OF THE INVENTION

The present invention relates to an organic solar cell with a photoactive region which comprises at least one organic donor material in contact with at least one organic acceptor material, wherein the donor material and the acceptor material form a donor-acceptor heterojunction and wherein the photoactive region comprises at least one substituted perylene.

### STATE OF THE ART

Periflanthene derivatives, i.e. compounds with a diindeno[1,2,3-cd:1′,2′,3′-Im]perylene base skeleton:

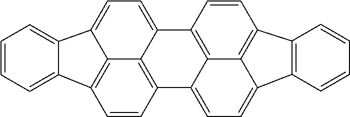

are the subject of intensive studies and have found use, for example, as chromophores and in systems based on electroluminescence, such as organic light-emitting diodes (OLEDs).

The synthesis and the electroluminescence of dibenzotetraphenylperiflanthene is described by J. D. Debad, J. C. Morris, V. Lynch, P. Magnus and A. J. Bard in J. Am. Chem. Soc. 1996, 118, pages 2374-2379.

M. Wehmeier, M. Wagner and K. Müllen describe, in Chem. Eur. J. 2001, 7, no. 10, p. 2197-2205, the preparation of novel perylene chromophores, including:

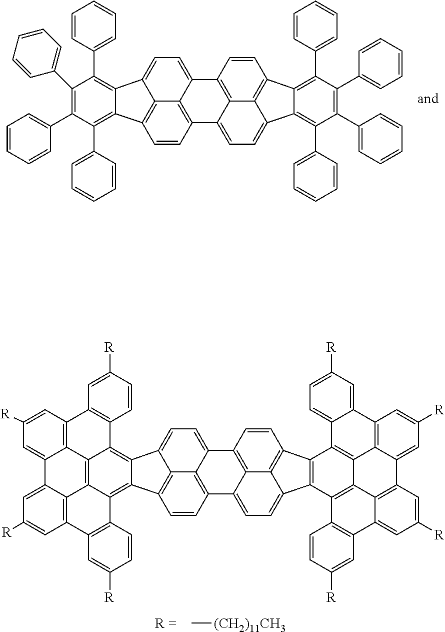

Owing to diminishing fossil raw materials and the CO₂ which is formed in the combustion of these raw materials and is active as a greenhouse gas, direct energy generation from sunlight is playing an increasing role. “Photovoltaics” is understood to mean the direct conversion of radiative energy, principally solar energy, to electrical energy.

In contrast to inorganic solar cells, the light does not directly generate free charge carriers in organic solar cells, but rather excitons are formed first, i.e. electrically neutral excited states in the form of electron-hole pairs. These excitons can be separated only by very high electrical fields or at suitable interfaces. In organic solar cells, sufficiently high fields are unavailable, and so all existing concepts for organic solar cells are based on exciton separation at photoactive interfaces (organic donor-acceptor interfaces or interfaces to an inorganic semiconductor). For this purpose, it is necessary that excitons which have been generated in the volume of the organic material can diffuse to this photoactive interface. The diffusion of excitons to the active interface thus plays a critical role in organic solar cells. In order to make a contribution to the photocurrent, the exciton diffusion length in a good organic solar cell must at least be in the order of magnitude of the typical penetration depth of light, in order that the predominant portion of the light can be utilized. The efficiency of an organic solar cell is characterized by its open-circuit voltage V_{OC}. Further important characteristics are the short-circuit current I_{SC}, the fill factor FF and the resulting efficiency η.

The first organic solar cell with an efficiency in the percent range was described by Tang et al. in 1986 (C W. Tang et al., Appl. Phys. Lett. 48, 183 (1986)). It consisted of a two-layer system with copper phthalocyanine (CuPc) as the p-semiconductor and perylene-3,4:9,10-tetracarboxylic acid bisimidazole (PTCBI) as the n-semiconductor.

Since then, there has been no lack of attempts to improve the efficiency of organic solar cells. Some approaches to the achievement or improvement of the properties of organic solar cells are listed below:
The use of an exciton-blocking layer, for example of bathocuproin.
One of the contact metals used has a large work function and the other a small work function, such that a Schottky barrier is formed by the organic layer.
Various dopants serve, inter alia, to improve the transport properties.
Arrangement of a plurality of individual solar cells so as to form a so-called tandem cell, which can be improved further, for example, by using p-i-n structures with doped transport layers of large band gap.

Instead of increasing the exciton diffusion length, it is alternatively also possible to reduce the mean distance to the next interface. To this end, it is possible to use mixed layers composed of donors and acceptors which form an interpenetrating network in which internal donor-acceptor heterojunctions are possible. S. Ushida et al. describe, in Appl. Phys. Lett., Vol. 84, no. 21, p. 4218-4220, an organic solar cell which was obtained by vacuum deposition of CuPc/C₆₀ to obtain a specific form of the mixed layer in the form of a donor-acceptor bulk heterojunction (BHJ). The advantage of such a mixed layer is that the excitons generated only have to cover a very short distance before they arrive at a domain boundary where they are separated. In spite of the above-described advantages, a critical factor for the bulk heterojunction (BHJ) is to find suitable materials and production processes which lead to mixed layers which have continuous transport pathways for both electrons and holes to their particular contacts. Since the individual materials each make up only a portion of the mixed layer, the transport properties for the charge carriers additionally in many cases deteriorate significantly compared to the pure layers. In addition, there are substance classes, for example particular oligothiophenes, which are surprisingly completely unsuitable for use in BHJ cells. One possible cause might be that these molecules mix too well with the second semiconductor material used to produce the mixed layer and therefore do not form any percolation pathways. However, there is currently still no demonstrable explanation. It is therefore virtually unforeseeable whether a particular electron or hole conductor material is suitable at all, and certainly not whether it is advantageously suitable for use in an organic solar cell with photoactive donor-acceptor transitions in the form of a bulk heterojunction.

JP 2008-135540 describes the use of perylene derivatives of the general formula

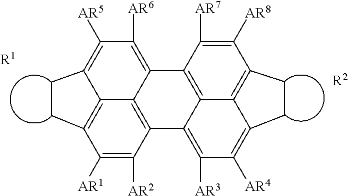

where R¹ and R² are each fused rings which may be substituted by alkyl, alkenyl, aryl, aralkyl or heterocyclyl, and AR¹-AR⁸ may each be alkyl, alkenyl, aryl, aralkyl or heterocyclyl, as an electron donor material for producing organic solar cells. In the examples, exclusively dibenzotetraphenylperiflanthene is used to produce an organic solar cell with photoactive donor-acceptor transitions in the form of a flat heterojunction. The use of differently substituted periflanthenes is not demonstrated, nor is the production of organic solar cells with donor-acceptor transitions in the form of a bulk heterojunction.

WO2010/031833 describes the use of dibenzotetraphenylperiflanthene of the formula

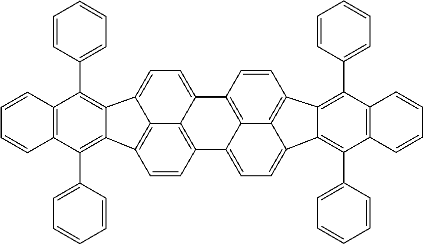

as an electron donor material in an organic solar cell with photoactive donor-acceptor transitions in the form of a bulk heterojunction.

European patent application 09164587.9 (WO 2011/000939), which was yet to be published at the priority date of the present application, describes an organic solar cell with a photoactive region which comprises at least one organic donor material in contact with at least one organic acceptor material, wherein the donor material and the acceptor material form a donor-acceptor heterojunction and wherein the photoactive region comprises at least one substituted periflanthene of the formula (A)

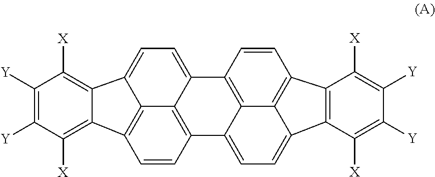

in which
X is independently selected from hydrogen and in each case unsubstituted or substituted alkyl, aryl, heteroaryl or oligo(het)aryl,
Y is independently selected from in each case unsubstituted or substituted alkyl, aryl, heteroaryl or oligo(het)aryl,
   where in each case at least two adjacent radicals, selected from the X and Y radicals, together with the carbon atoms of the benzene ring to which they are bonded, may also be a fused ring system having 1, 2, 3, 4, 5, 6, 7 or 8 further rings.

It is an object of the invention to provide an organic solar cell in which the efficiency of energy conversion is improved.

It has now been found that, surprisingly, substituted periflanthenes are particularly advantageously suitable as semiconductor materials for producing organic solar cells with photoactive donor-acceptor transitions, especially with photoactive donor-acceptor transitions in the form of a bulk heterojunction. They serve here especially as donor materials (p-semiconductors).

### SUMMARY OF THE INVENTION

The invention first provides an organic solar cell with a photoactive region which comprises at least one organic donor material in contact with at least one organic acceptor material, wherein the donor material and the acceptor material form a donor-acceptor heterojunction and wherein the photoactive region comprises at least one substituted perylene of the formula (I)

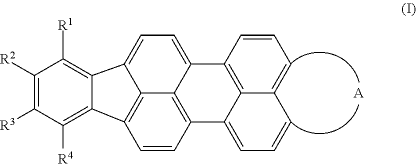

in which
R¹ and R⁴ are independently selected from hydrogen and in each case unsubstituted alkyl, substituted alkyl, unsubstituted aryl, substituted aryl, unsubstituted heteroaryl, substituted heteroaryl, unsubstituted oligo(het)aryl and substituted oligo(het)aryl;
R² and R³ are independently selected from hydrogen and in each case unsubstituted alkyl, substituted alkyl, unsubstituted aryl, substituted aryl, unsubstituted heteroaryl, substituted heteroaryl, unsubstituted oligo(het)aryl and substituted oligo(het)aryl;
   where in each case at least two adjacent radicals, selected from the R¹, R², R³ and R⁴ radicals, together with the carbon atoms of the benzene ring to which they are bonded, may also be a fused ring system having 1, 2, 3, 4, 5, 6, 7 or 8 further rings, and
A together with the carbon atoms to which it is bonded is a fused monocyclic, dicyclic, tricyclic, tetracyclic, pentacyclic or hexacyclic ring system having at least one exocyclic keto group, where the ring system optionally bears one or more substituents bonded via a single bond.

### DESCRIPTION OF FIGURES

FIG. 1 shows a solar cell suitable for the use of substituted perylenes of the formula (I) and having normal structure.

FIG. 2 shows a solar cell with inverse structure.

FIG. 3 shows the structure of a solar cell with normal structure and with a donor-acceptor interface in the form of a bulk heterojunction.

FIG. 4 shows the structure of a solar cell with inverse structure and with a donor-acceptor interface in the form of a bulk heterojunction.

FIG. 5 shows the structure of a tandem cell.

FIG. 6 shows the structure of a solar cell with a donor-acceptor interface in the form of a bulk heterojunction configured as a gradient.

### DESCRIPTION OF THE INVENTION

In the context of the invention, the expression “unsubstituted or substituted alkyl, aryl, heteroaryl or oligo(het)aryl” represents unsubstituted or substituted alkyl, unsubstituted or substituted aryl, unsubstituted or substituted heteroaryl or unsubstituted or substituted oligo(het)aryl.

In the context of the present invention, the expression “alkyl” comprises straight-chain or branched alkyl. Alkyl is preferably C₁-C₃₀-alkyl, especially C₁-C₂₀-alkyl and most preferably C₁-C₁₂-alkyl. Examples of alkyl groups are especially methyl, ethyl, n-propyl, isopropyl, n-butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl.

The expression “alkyl” also comprises alkyl radicals whose carbon chains may be interrupted by one or more nonadjacent groups selected from —O—, —S—, —NR^{d}—, —C(═O)—, —S(═O)— and/or —S(═O)₂—. R^{d} is preferably hydrogen, alkyl, cycloalkyl, heterocycloalkyl, aryl or hetaryl.

Substituted alkyl groups may, depending on the length of the alkyl chain, have one or more (e.g. 1, 2, 3, 4, 5 or more than 5) substituents. These are preferably each independently selected from cycloalkyl, heterocycloalkyl, aryl, hetaryl, fluorine, chlorine, bromine, cyano and nitro.

Aryl-substituted alkyl radicals (“aralkyl” or arylalkyl) have at least one unsubstituted or substituted aryl group, as defined below. The alkyl group in “aralkyl” may bear at least one further substituent and/or be interrupted by one or more nonadjacent groups selected from —O—, —S—, —NR^{c}—, —C(═O)—, —S(═O)— and/or —S(═O)₂—. R^{c} is preferably hydrogen, alkyl, cycloalkyl, heterocycloalkyl, aryl or hetaryl. Arylalkyl is preferably phenyl-C₁-C₁₀-alkyl, more preferably phenyl-C₁-C₄-alkyl, for example benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl, 4-phenbut-2-yl, 1-(phenmeth)eth-1-yl, 1-(phenmethyl)-1-(methyl)eth-1-yl or 1-(phenmethyl)-1-(methyl)prop-1-yl; preferably benzyl and 2-phenethyl.

Halogen-substituted alkyl groups (“haloalkyl”) comprise a straight-chain or branched alkyl group in which at least one hydrogen atom or all hydrogen atoms are replaced by halogen. The halogen atoms are preferably selected from fluorine, chlorine and bromine, especially fluorine and chlorine. Examples of haloalkyl groups are especially chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, —CH₂—C₂F₅, —CF₂—C₂F₅, —CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl.

The above remarks regarding unsubstituted or substituted alkyl also apply to unsubstituted or substituted alkoxy, unsubstituted or substituted alkylamino, unsubstituted or substituted alkylthio, etc.

In the context of the invention, “cycloalkyl” denotes a cycloaliphatic radical having preferably 3 to 10, more preferably 5 to 8, carbon atoms. Examples of cycloalkyl groups are especially cyclopropyl, cyclobutyl, cyclopentyl, cyclohexyl, cycloheptyl or cyclooctyl.

Substituted cycloalkyl groups may, depending on the ring size, have one or more (e.g. 1, 2, 3, 4, 5 or more than 5) substituents. These are preferably each independently selected from alkyl, alkoxy, alkylamino, alkylthio, cycloalkyl, heterocycloalkyl, aryl, hetaryl, fluorine, chlorine, bromine, cyano and nitro. In the case of substitution, the cycloalkyl groups preferably bear one or more, for example one, two, three, four or five, C₁-C₆-alkyl groups. Examples of substituted cycloalkyl groups are especially 2- and 3-methylcyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-sec.-butylcyclohexyl, 2-, 3- and 4-tert-butyl-cyclohexyl, 2-, 3- and 4-methylcycloheptyl, 2-, 3- and 4-ethylcycloheptyl, 2-, 3- and 4-propylcycloheptyl, 2-, 3- and 4-isopropylcycloheptyl, 2-, 3- and 4-butylcycloheptyl, 2-, 3- and 4-sec-butylcycloheptyl, 2-, 3- and 4-tert-butylcycloheptyl, 2-, 3-, 4- and 5-methyl-cyclooctyl, 2-, 3-, 4- and 5-ethylcyclooctyl, 2-, 3-, 4- and 5-propylcyclooctyl.

In the context of the present invention, the expression “aryl” comprises mono- or polycyclic aromatic hydrocarbon radicals, having 6 to 18, preferably 6 to 14, more preferably 6 to 10, carbon atoms. Examples of aryl are especially phenyl, naphthyl, indenyl, fluorenyl, anthracenyl, phenanthrenyl, naphthacenyl, chrysenyl, pyrenyl, etc., and especially phenyl or naphthyl.

Substituted aryls may, depending on the number and size of their ring systems, have one or more (e.g. 1, 2, 3, 4, 5 or more than 5) substituents. These are preferably each independently selected from alkyl, alkoxy, alkylamino, alkylthio, cycloalkyl, heterocycloalkyl, aryl, hetaryl, fluorine, chlorine, bromine, cyano and nitro. The alkyl, alkoxy, alkylamino, alkylthio, cycloalkyl, heterocycloalkyl, aryl and hetaryl substituents on the aryl may in turn be unsubstituted or substituted. Reference is made to the substituents mentioned above for these groups. The substituents on the aryl are preferably selected from alkyl, alkoxy, haloalkyl, haloalkoxy, aryl, fluorine, chlorine, bromine, cyano and nitro. Substituted aryl is more preferably substituted phenyl which generally bears 1, 2, 3, 4 or 5, preferably 1, 2 or 3, substituents.

Substituted aryl is preferably aryl substituted by at least one alkyl group (“alkaryl”). Alkaryl groups may, depending on the size of the aromatic ring system, have one or more (e.g. 1, 2, 3, 4, 5, 6, 7, 8, 9 or more than 9) alkyl substituents. The alkyl substituents may be unsubstituted or substituted. Each alkyl substituent may have 1 to 30 carbon atoms. In this regard, reference is made to the above statements regarding unsubstituted and substituted alkyl. In a preferred embodiment, the alkaryl groups have exclusively unsubstituted alkyl substituents. Alkaryl is preferably phenyl which bears 1, 2, 3, 4 or 5, preferably 1, 2 or 3, more preferably 1 or 2, alkyl substituents having 1 to 30 carbon atoms.

Aryl which bears one or more radicals is, for example, 2-, 3- and 4-methylphenyl, 2,4-, 2,5-, 3,5- and 2,6-dimethylphenyl, 2,4,6-trimethylphenyl, 2-, 3- and 4-ethylphenyl, 2,4-, 2,5-, 3,5- and 2,6-diethylphenyl, 2,4,6-triethylphenyl, 2-, 3- and 4-propylphenyl, 2,4-, 2,5-, 3,5- and 2,6-dipropylphenyl, 2,4,6-tripropylphenyl, 2-, 3- and 4-isopropylphenyl, 2,4-, 2,5-, 3,5- and 2,6-diisopropylphenyl, 2,4,6-triisopropylphenyl, 2-, 3- and 4-butylphenyl, 2,4-, 2,5-, 3,5- and 2,6-dibutylphenyl, 2,4,6-tributylphenyl, 2-, 3- and 4-isobutylphenyl, 2,4-, 2,5-, 3,5- and 2,6-diisobutylphenyl, 2,4,6-triisobutylphenyl, 2-, 3- and 4-sec-butylphenyl, 2,4-, 2,5-, 3,5- and 2,6-di-sec-butylphenyl, 2,4,6-tri-sec-butylphenyl, 2-, 3- and 4-tert-butylphenyl, 2,4-, 2,5-, 3,5- and 2,6-di-tert-butylphenyl and 2,4,6-tri-tert-butylphenyl; 2-, 3- and 4-methoxyphenyl, 2,4-, 2,5-, 3,5- and 2,6-dimethoxyphenyl, 2,4,6-trimethoxyphenyl, 2-, 3- and 4-ethoxyphenyl, 2,4-, 2,5-, 3,5- and 2,6-diethoxyphenyl, 2,4,6-triethoxyphenyl, 2-, 3- and 4-propoxyphenyl, 2,4-, 2,5-, 3,5- and 2,6-dipropoxyphenyl, 2-, 3- and 4-isopropoxyphenyl, 2,4-, 2,5-, 3,5- and 2,6-diisopropoxyphenyl and 2-, 3- and 4-butoxyphenyl; 2-, 3- and 4-cyanophenyl.

The above remarks regarding unsubstituted or substituted aryl also apply to unsubstituted or substituted aryloxy and unsubstituted or substituted arylthio. Examples of aryloxy are phenoxy and naphthyloxy.

In the context of the present invention, the expression “heterocycloalkyl” comprises nonaromatic, unsaturated or fully saturated, cycloaliphatic groups having generally 5 to 8 ring atoms, preferably 5 or 6 ring atoms. In the heterocycloalkyl groups, compared to the corresponding cycloalkyl groups, 1, 2, 3, 4 or more than 4 of the ring carbon atoms are replaced by heteroatoms or heteroatom-containing groups. The heteroatoms or heteroatom-containing groups are preferably selected from —O—, —S—, —NR^{f}', —C(═O)—, —S(═O)— and/or —S(═O)₂—. R^{f} is preferably hydrogen, alkyl, cycloalkyl, heterocycloalkyl, aryl or hetaryl. Examples of heterocycloalkyl groups are especially pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, dihydrothien-2-yl, tetrahydrofuranyl, dihydrofuran-2-yl, tetrahydropyranyl, 1,2-oxazolin-5-yl, 1,3-oxazolin-2-yl and dioxanyl.

Substituted heterocycloalkyl groups may, depending on the ring size, have one or more (e.g. 1, 2, 3, 4, 5 or more than 5) substituents. These are preferably each independently selected from alkyl, alkoxy, alkylamino, alkylthio, cycloalkyl, heterocycloalkyl, aryl, hetaryl, fluorine, chlorine, bromine, cyano and nitro. In the case of substitution, the heterocycloalkyl groups preferably bear one or more, for example one, two, three, four or five, C₁-C₆-alkyl groups, e.g. 2,2,6,6-tetramethylpiperidinyl.

In the context of the present invention, the expression “heteroaryl” (hetaryl) comprises heteroaromatic, mono- or polycyclic groups. In addition to the ring carbon atoms, these have 1, 2, 3, 4 or more than 4 of the ring heteroatoms. The ring heteroatoms are preferably selected from oxygen, nitrogen, selenium and sulfur. The hetaryl groups have preferably 5 to 18, e.g. 5, 6, 8, 9, 10, 11, 12, 13 or 14, ring atoms.

Monocyclic hetaryl groups are preferably 5- or 6-membered hetaryl groups, such as 2-furyl(furan-2-yl), 3-furyl(furan-3-yl), 2-thienyl(thiophen-2-yl), 3-thienyl(thiophen-3-yl), selenophen-2-yl, selenophen-3-yl, 1H-pyrrol-2-yl, 1H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

Polycyclic hetaryl has 2, 3, 4 or more than 4 fused rings. The fused-on rings may be aromatic, saturated or partly unsaturated. Examples of polycyclic hetaryl groups are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl; benzothiadiazolyl, benzoxazinyl, benzopyrazolyl, benzimidazolyl, benzotriazolyl, benzotriazinyl, benzoselenophenyl, thienothiophenyl, thienopyrimidyl, thiazolothiazolyl, dibenzopyrrolyl(carbazolyl), dibenzofuranyl, dibenzothiophenyl, naphtho[2,3-b]-thiophenyl, naphtha[2,3-b]furyl, dihydroindolyl, dihydroindolizinyl, dihydroisoindolyl, dihydroquinolinyl, dihydroisoquinolinyl.

Substituted heteroaryls may, depending on the number and size of their ring systems, have one or more (e.g. 1, 2, 3, 4, 5 or more than 5) substituents. These are preferably each independently selected from alkyl, alkoxy, alkylamino, alkylthio, cycloalkyl, heterocycloalkyl, aryl, hetaryl, fluorine, chlorine, bromine, cyano and nitro. Halogen substituents are preferably fluorine, chlorine or bromine. The substituents are preferably selected from C₁-C₆-alkyl, C₁-C₆-alkoxy, hydroxyl, carboxyl, halogen and cyano.

The above remarks regarding unsubstituted or substituted heteroaryl also apply to unsubstituted or substituted heteroaryloxy and unsubstituted or substituted heteroarylthio.

In the context of the present application, the expression “oligo(het)aryl” denotes unsubstituted or substituted groups having at least two repeat units. The repeat units may all have the same definition, some of the repeat units may have different definitions or all repeat units may have different definitions. The repeat unit is selected from aryldiyl groups, hetaryldiyl groups and combinations thereof. The aryldiyl group is a divalent group derived from an aromatic, preferably a group derived from benzene or naphthalene, such as 1,2-phenylene (o-phenylene), 1,3-phenylene (m-phenylene), 1,4-phenylene (p-phenylene), 1,2-naphthylene, 2,3-naphthylene, 1,4-naphthylene, etc. The hetaryldiyl group is a divalent group derived from a heteroaromatic, preferably a group derived from thiophene or furan. The terminal group of the oligo(het)aryl groups is a monovalent group. This preferably likewise derives from the aforementioned repeat units. The oligo(het)aryl groups may be unsubstituted or substituted. Substituted oligo(het)aryls may, depending on the number and size of their ring systems, have one or more (e.g. 1, 2, 3, 4, 5, 6, 7, 8, 9 or more than 9) substituents. These substituents are preferably each independently selected from unsubstituted alkyl, haloalkyl, fluorine or chlorine.

Suitable repeat units are as follows:

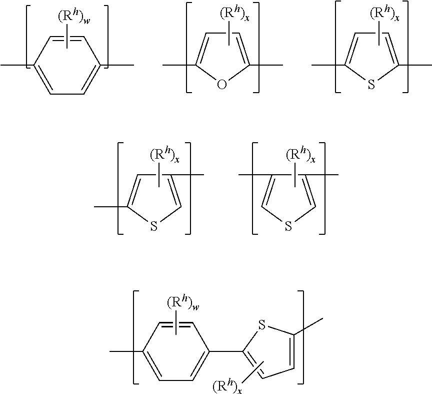

in which the R^{h} radicals are each independently unsubstituted alkyl, haloalkyl, fluorine or chlorine, w is 0, 1, 2, 3 or 4 and x is 0, 1 or 2.

Preferred oligoaryl groups are biphenylyl, p-terphenylyl, m-terphenylyl, o-terphenylyl, quaterphenylyl, e.g. p-quaterphenylyl, quinquephenylyl, e.g. p-quinquephenylyl.

Examples of oligohetaryl groups are

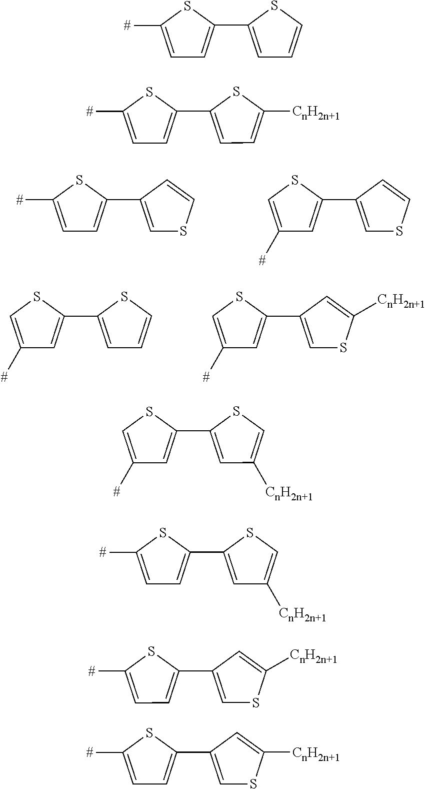

in which # represents a bonding site to the rest of the molecule. n is 1, 2, 3, 4, 5, 6, 7 or 8.

Halogen represents fluorine, chlorine, bromine or iodine.

Specific examples of the R¹, R², R³, R⁴, R^{I}, R^{II}, and R⁵ to R²⁹ radicals specified in the above formula (I) and the formulae which follow are:

methyl, ethyl, n-propyl, isopropyl, n-butyl, isobutyl, sec-butyl, tert-butyl, n-pentyl, n-hexyl, n-heptyl, n-octyl, n-nonyl, n-decyl, n-undecyl, n-dodecyl, n-tridecyl, n-tetradecyl, n-hexadecyl, n-octadecyl and n-eicosyl, 2-methoxyethyl, 2-ethoxyethyl, 2-propoxyethyl, 2-butoxyethyl, 3-methoxypropyl, 3-ethoxypropyl, 3-propoxypropyl, 3-butoxypropyl, 4-methoxybutyl, 4-ethoxybutyl, 4-propoxybutyl, 3,6-dioxaheptyl, 3,6-dioxaoctyl, 4,8-dioxanonyl, 3,7-dioxaoctyl, 3,7-dioxanonyl, 4,7-dioxaoctyl, 4,7-dioxanonyl, 2- and 4-butoxybutyl, 4,8-dioxadecyl, 3,6,9-trioxadecyl, 3,6,9-trioxaundecyl, 3,6,9-trioxadodecyl, 3,6,9,12-tetraoxamidecyl and 3,6,9,12-tetraoxatetradecyl;
2-methylthioethyl, 2-ethylthioethyl, 2-propylthioethyl, 2-butylthioethyl, 3-methylthiopropyl, 3-ethylthiopropyl, 3-propylthiopropyl, 3-butylthiopropyl, 4-methylthiobutyl, 4-ethylthiobutyl, 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiamidecyl and 3,6,9,12-tetrathiatetradecyl;
2-monomethyl- and 2-monoethylaminoethyl, 2-dimethylaminoethyl, 2- and 3-dimethylaminopropyl, 3-monoisopropylaminopropyl, 2- and 4-monopropylaminobutyl, 2- and 4-dimethylaminobutyl, 6-methyl-3,6-diazaheptyl, 3,6-dimethyl-3,6-diazaheptyl, 3,6-diazaoctyl, 3,6-dimethyl-3,6-diazaoctyl, 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazamidecyl and 3,6,9,12-tetramethyl-3,6,9,12-tetraazamidecyl;
(1-ethylethylidene)aminoethylene, (1-ethylethylidene)aminopropylene, (1-ethylethylidene)aminobutylene, (1-ethylethylidene)aminodecylene and (1-ethylethylidene)aminododecylene;
propan-2-on-1-yl, butan-3-on-1-yl, butan-3-on-2-yl and 2-ethylpentan-3-on-1-yl;
2-methylsulfinylethyl, 2-ethylsulfinylethyl, 2-propylsulfinylethyl, 2-isopropylsulfinylethyl, 2-butylsulfinylethyl, 2- and 3-methylsulfinylpropyl, 2- and 3-ethylsulfinylpropyl, 2- and 3-propylsulfinylpropyl, 2- and 3-butylsulfinylpropyl, 2- and 4-methylsulfinylbutyl, 2- and 4-ethylsulfinylbutyl, 2- and 4-propylsulfinylbutyl and 4-butylsulfinylbutyl;
2-methylsulfonylethyl, 2-ethylsulfonylethyl, 2-propylsulfonylethyl, 2-isopropylsulfonylethyl, 2-butylsulfonylethyl, 2- and 3-methylsulfonylpropyl, 2- and 3-ethylsulfonylpropyl, 2- and 3-propylsulfonylpropyl, 2- and 3-butylsulfonylproypl, 2- and 4-methylsulfonylbutyl, 2- and 4-ethylsulfonylbutyl, 2- and 4-propylsulfonylbutyl and 4-butylsulfonylbutyl;
carboxymethyl, 2-carboxyethyl, 3-carboxypropyl, 4-carboxybutyl, 5-carboxypentyl, 6-carboxyhexyl, 8-carboxyoctyl, 10-carboxydecyl, 12-carboxydodecyl and 14-carboxyl-tetradecyl;
sulfomethyl, 2-sulfoethyl, 3-sulfopropyl, 4-sulfobutyl, 5-sulfopentyl, 6-sulfohexyl, 8-sulfooctyl, 10-sulfodecyl, 12-sulfododecyl and 14-sulfotetradecyl;
2-hydroxyethyl, 2- and 3-hydroxypropyl, 3- and 4-hydroxybutyl and 8-hydroxyl-4-oxaoctyl;
2-cyanoethyl, 3-cyanopropyl, 3- and 4-cyanobutyl;
2-chloroethyl, 2- and 3-chloropropyl, 2-, 3- and 4-chlorobutyl, 2-bromoethyl, 2- and 3-bromopropyl and 2-, 3- and 4-bromobutyl;
2-nitroethyl, 2- and 3-nitropropyl and 2-, 3- and 4-nitrobutyl; methoxy, ethoxy, propoxy, butoxy, pentoxy and hexoxy;
methylthio, ethylthio, propylthio, butylthio, pentylthio and hexylthio;
methylamino, ethylamino, propylamino, butylamino, pentylamino, hexylamino, dicyclopentylamino, dicyclohexylamino, dicycloheptylamino, diphenylamino and dibenzylamino;
formylamino, acetylamino, propionylamino and benzoylamino;
carbamoyl, methylaminocarbonyl, ethylaminocarbonyl, propylaminocarbonyl, butyl-aminocarbonyl, pentylaminocarbonyl, hexylaminocarbonyl, heptylaminocarbonyl, octylaminocarbonyl, nonylaminocarbonyl, decylaminocarbonyl and phenylamino-carbonyl;
aminosulfonyl, n-dodecylaminosulfonyl, N,N-diphenylaminosulfonyl, and N,N-bis(4-chlorophenyl)aminosulfonyl;
methoxycarbonyl, ethoxycarbonyl, propoxycarbonyl hexoxycarbonyl, dodecyloxycarbonyl, octadecyloxycarbonyl, phenoxycarbonyl, (4-tert-butylphenoxy)carbonyl and (4-chlorophenoxy)carbonyl;
methoxysulfonyl, ethoxysulfonyl, propoxysulfonyl, butoxysulfonyl, hexoxysulfonyl, dodecyloxysulfonyl, octadecyloxysulfonyl, phenoxysulfonyl, 1- and 2-naphthyloxysulfonyl, (4-tert-butylphenoxy)sulfonyl and (4-chlorophenoxy)sulfonyl;
fluorine, chlorine, bromine and iodine;
cyclopropyl, cyclobutyl, cyclopentyl, 2- and 3-methylcyclopentyl, 2- and 3-ethylcyclo-pentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, cycloheptyl, 2-, 3- and 4-methyl-cycloheptyl, 2-, 3- and 4-ethylcycloheptyl, 3- and 4-propylcycloheptyl, 3- and 4-iso-propylcycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethylcyclooctyl and 3-, 4- and 5-propylcyclooctyl; 3- and 4-hydroxycyclohexyl, 3- and 4-nitrocyclohexyl and 3- and 4-chlorocyclohexyl;
1-, 2- and 3-cyclopentenyl, 1-, 2-, 3- and 4-cyclohexenyl, 1-, 2- and 3-cycloheptenyl and 1-, 2-, 3- and 4-cyclooctenyl;
2-dioxanyl, 4-morpholinyl, 4-thiomorpholinyl, 2- and 3-tetrahydrofuryl, 1-, 2- and 3-pyrrolidinyl, 1-piperazyl, 1-diketopiperazyl and 1-, 2-, 3- and 4-piperidyl;
phenyl, 2-naphthyl, 2- and 3-pyrryl, 2-, 3- and 4-pyridyl, 2-, 4- and 5-pyrimidyl, 3-, 4- and 5-pyrazolyl, 2-, 4- and 5-imidazolyl, 2-, 4- and 5-thiazolyl, 3-(1,2,4-triazyl), 2-(1,3,5-triazyl), 6-quinaldyl, 3-, 5-, 6- and 8-quinolinyl, 2-benzoxazolyl, 2-benzothiazolyl, 5-benzothiadiazolyl, 2- and 5-benzimidazolyl and 1- and 5-isoquinolyl;
1-, 2-, 3-, 4-, 5-, 6- and 7-indolyl, 1-, 2-, 3-, 4-, 5-, 6- and 7-isoindolyl, 5-(4-methylisoindolyl), 5-(4-phenylisoindolyl), 1-, 2-, 4-, 6-, 7- and 8-(1,2,3,4-tetrahydroisoquinolinyl), 3-(5-phenyl)-(1,2,3,4-tetrahydroisoquinolinyl), 5-(3-dodecyl-(1,2,3,4-tetrahydroisoquinolinyl), 1-, 2-, 3-, 4-, 5-, 6-, 7- and 8-(1,2,3,4-tetrahydroquinolinyl) and 2-, 3-, 4-, 5-, 6-, 7- and 8-chromanyl, 2-, 4- and 7-quinolinyl, 2-(4-phenylquinolinyl) and 2-(5-ethylquinolinyl);
2-, 3- and 4-methylphenyl, 2,4-, 3,5- and 2,6-dimethylphenyl, 2,4,6-trimethylphenyl, 2-, 3- and 4-ethylphenyl, 2,4-, 3,5- and 2,6-diethylphenyl, 2,4,6-triethylphenyl, 2-, 3- and 4-propylphenyl, 2,4-, 3,5- and 2,6-dipropylphenyl, 2,4,6-tripropylphenyl, 2-, 3- and 4-isopropylphenyl, 2,4-, 3,5- and 2,6-diisopropylphenyl, 2,4,6-triisopropylphenyl, 2-, 3- and 4-butylphenyl, 2,4-, 3,5- and 2,6-dibutylphenyl, 2,4,6-tributylphenyl, 2-, 3- and 4-isobutylphenyl, 2,4-, 3,5- and 2,6-diisobutylphenyl, 2,4,6-triisobutylphenyl, 2-, 3- and 4-sec-butylphenyl, 2,4-, 3,5- and 2,6-di-sec-butylphenyl and 2,4,6-tri-sec-butylphenyl; 2-, 3- and 4-methoxyphenyl, 2,4-, 3,5- and 2,6-dimethoxyphenyl, 2,4,6-trimethoxy-phenyl, 2-, 3- and 4-ethoxyphenyl, 2,4-, 3,5- and 2,6-diethoxyphenyl, 2,4,6-triethoxyphenyl, 2-, 3- and 4-propoxyphenyl, 2,4-, 3,5- and 2,6-dipropoxyphenyl, 2-, 3- and 4-isopropoxyphenyl, 2,4- and 2,6-diisopropoxyphenyl and 2-, 3- and 4-butoxy-phenyl; 2-, 3- and 4-chlorophenyl and 2,4-, 3,5- and 2,6-dichlorophenyl; 2-, 3- and 4-hydroxyphenyl and 2,4-, 3,5- and 2,6-dihydroxyphenyl; 2-, 3- and 4-cyanophenyl; 3- and 4-carboxyphenyl; 3- and 4-carboxamidophenyl, 3- and 4-N-methylcarboxamido-phenyl and 3- and 4-N-ethylcarboxamidophenyl; 3- and 4-acetylaminophenyl, 3- and 4-propionylaminophenyl and 3- and 4-butyrylaminophenyl; 3- and 4-N-phenylamino-phenyl, 3- and 4-N-(o-tolyl)aminophenyl, 3- and 4-N-(m-tolyl)aminophenyl and 3- and 4-(p-tolyl)aminophenyl; 3- and 4-(2-pyridyl)aminophenyl, 3- and 4-(3-pyridyl)aminophenyl, 3- and 4-(4-pyridyl)aminophenyl, 3- and 4-(2-pyrimidyl)aminophenyl and 4-(4-pyrimidyl)aminophenyl;
4-phenylazophenyl, 4-(1-naphthylazo)phenyl, 4-(2-naphthylazo)phenyl, 4-(4-naphthylazo)phenyl, 4-(2-pyriylazo)phenyl, 4-(3-pyridylazo)phenyl, 4-(4-pyridylazo)phenyl, 4-(2-pyrimidylazo)phenyl, 4-(4-pyrimidylazo)phenyl and 4-(5-pyrimidylazo)phenyl;
phenoxy, phenylthio, 2-naphthoxy, 2-naphthylthio, 2-, 3- and 4-pyridyloxy, 2-, 3- and 4-pyridylthio, 2-, 4- and 5-pyrimidyloxy and 2-, 4- and 5-pyrimidylthio.

Specific preferred fluorinated R¹, R², R³, R⁴, R^{I}, R^{II}, and R⁵ to R²⁹ radicals are as follows:

2,2,2-trifluoroethyl, 2,2,3,3,3-pentafluoropropyl, 2,2-difluoroethyl, 2,2,3,3,4,4,4-hepta-fluorobutyl, 2,2,3,3,3-pentafluoropropyl, 1H,1H-pentadecafluorooctyl, 3-bromo-3,3-difluoropropyl, 3,3,3-trifluoropropyl, 3,3,3-trifluoropropyl, 1H,1H,2H,2H-perfluorodecyl, 3-(perfluorooctyl)propyl, 4,4-difluorobutyl, 4,4,4-trifluorobutyl, 5,5,6,6,6-penta-fluorohexyl, 2,2-difluoropropyl, 2,2,2-trifluoro-1-phenylethylamino, 1-benzyl-2,2,2-trifluoroethyl, 2-bromo-2,2-difluoroethyl, 2,2,2-trifluoro-1-pyridin-2-ylethyl, 2,2-difluoro-propyl, 2,2,2-trifluoro-1-(4-methoxyphenyl)ethylamino, 2,2,2-trifluoro-1-phenylethyl, 2,2-difluoro-1-phenylethyl, 1-(4-bromophenyl)-2,2,2-trifluoroethyl, 3-bromo-3,3-difluoropropyl, 3,3,3-trifluoropropylamino, 3,3,3-trifluoro-n-propyl, 1H,1H,2H,2H-perfluorodecyl, 3-(perfluorooctyl)propyl, pentafluorophenyl, 2,3,5,6-tetrafluorophenyl, 4-cyano(2,3,5,6)-tetrafluorophenyl, 4-carboxyl-2,3,5,6-tetrafluorophenyl, 2,4-difluorophenyl, 2,4,5-trifluorophenyl, 2,4,6-trifluorophenyl, 2,5-difluorophenyl, 2-fluoro-5-nitrophenyl, 2-fluoro-5-trifluoromethylphenyl, 2-fluoro-5-methylphenyl, 2,6-difluorophenyl, 4-carboxamido-2,3,5,6-tetrafluorophenyl, 2-bromo-4,6-difluorophenyl, 4-bromo-2-fluorophenyl, 2,3-difluorophenyl, 4-chloro-2-fluorophenyl, 2,3,4-trifluorophenyl, 2-fluoro-4-iodophenyl, 4-bromo-2,3,5,6-tetrafluorophenyl, 2,3,6-trifluorophenyl, 2-bromo-3,4,6-trifluorophenyl, 2-bromo-4,5,6-trifluorophenyl, 4-bromo-2,6-difluorophenyl, 2,3,4,5-tetrafluorophenyl, 2,4-difluoro-6-nitrophenyl, 2-fluoro-4-nitrophenyl, 2-chloro-6-fluorophenyl, 2-fluoro-4-methylphenyl, 3-chloro-2,4-difluorophenyl, 2,4-dibromo-6-fluorophenyl, 3,5-dichloro-2,4-difluorophenyl, 4-cyano-1-fluorophenyl, 1-chloro-4-fluorophenyl, 2-fluoro-3-trifluoromethylphenyl, 2-trifluoromethyl-6-fluorophenyl, 2,3,4,6-tetrafluorophenyl, 3-chloro-2-fluorophenyl, 5-chloro-2-fluorophenyl, 2-bromo-4-chloro-6-fluorophenyl, 2,3-dicyano-4,5,6-trifluorophenyl, 2,4,5-trifluoro-3-carboxyphenyl, 2,3,4-trifluoro-6-carboxyphenyl, 2,3,5-trifluorophenyl, 4-trifluoromethyl-2,3,5,6-tetrafluorophenyl, 1-fluoro-5-carboxyphenyl, 2-chloro-4,6-difluorophenyl, 6-bromo-3-chloro-2,4-difluorophenyl, 2,3,4-trifluoro-6-nitrophenyl, 2,5-difluoro-4-cyanophenyl, 2,5-difluoro-4-trifluoromethylphenyl, 2,3-difluoro-6-nitrophenyl, 4-trifluoromethyl-2,3-difluorophenyl, 2-bromo-4,6-difluorophenyl, 4-bromo-2-fluorophenyl, 2-nitrotetrafluorophenyl, 2,2′,3,3′,4′,5,5′,6,6′-nonafluorobiphenyl, 2-nitro-3,5,6-trifluorophenyl, 2-bromo-6-fluorophenyl, 4-chloro-2-fluoro-6-iodophenyl, 2-fluoro-6-carboxyphenyl, 2,4-difluoro-3-trifluorophenyl, 2-fluoro-4-trifluorophenyl, 2-fluoro-4-carboxyphenyl, 4-bromo-2,5-difluorophenyl, 2,5-dibromo-3,4,6-trifluorophenyl, 2-fluoro-5-methylsulfonylphenyl, 5-bromo-2-fluorophenyl, 2-fluoro-4-hydroxymethylphenyl, 3-fluoro-4-bromomethylphenyl, 2-nitro-4-trifluoromethylphenyl, 4-trifluoromethylphenyl, 2-bromo-4-trifluoromethylphenyl, 2-bromo-6-chloro-4-(trifluoromethyl)phenyl, 2-chloro-4-trifluoromethylphenyl, 3-nitro-4-(trifluoromethyl)-phenyl, 2,6-dichloro-4-(trifluoromethyl)phenyl, 4-trifluorophenyl, 2,6-dibromo-4-(trifluoromethyl)phenyl, 4-trifluoromethyl-2,3,5,6-tetrafluorophenyl, 3-fluoro-4-trifluoromethylphenyl, 2,5-difluoro-4-trifluoromethylphenyl, 3,5-difluoro-4-trifluoromethylphenyl, 2,3-difluoro-4-trifluoromethylphenyl, 2,4-bis(trifluoromethyl)-phenyl, 3-chloro-4-trifluoromethylphenyl, 2-bromo-4,5-di(trifluoromethyl)phenyl, 5-chloro-2-nitro-4-(trifluoromethyl)phenyl, 2,4,6-tris(trifluoromethyl)phenyl, 3,4-bis-(trifluoromethyl)phenyl, 2-fluoro-3-trifluoromethylphenyl, 2-iodo-4-trifluoromethylphenyl, 2-nitro-4,5-bis(trifluoromethyl)phenyl, 2-methyl-4-(trifluoromethyl)phenyl, 3,5-dichloro-4-(trifluoromethyl)phenyl, 2,3,6-trichloro-4-(trifluoromethyl)phenyl, 4-(trifluoromethyl)benzyl, 2-fluoro-4-(trifluoromethyl)benzyl, 3-fluoro-4-(trifluoromethyl)benzyl, 3-chloro-4-(trifluoromethyl)benzyl, 4-fluorophenethyl, 3-(trifluoromethyl)phenethyl, 2-chloro-6-fluorophenethyl, 2,6-dichlorophenethyl, 3-fluorophenethyl, 2-fluorophenethyl, (2-trifluoromethyl)phenethyl, 4-fluorophenethyl, 3-fluorophenethyl, 4-trifluoromethylphenethyl, 2,3-difluorophenethyl, 3,4-difluorophenethyl, 2,4-difluorophenethyl, 2,5-difluorophenethyl, 3,5-difluorophenethyl, 2,6-difluorophenethyl, 4-(4-fluorophenyl)phenethyl, 3,5-di(trifluoromethyl)phenethyl, pentafluorophenethyl, 2,4-di(trifluoromethyl)phenethyl, 2-nitro-4-(trifluoromethyl)-phenethyl, (2-fluoro-3-trifluoromethyl)phenethyl, (2-fluoro-5-trifluoromethyl)phenethyl, (3-fluoro-5-trifluoromethyl)phenethyl, (4-fluoro-2-trifluoromethyl)phenethyl, (4-fluoro-3-trifluoromethyl)phenethyl, (2-fluoro-6-trifluoromethyl)phenethyl, (2,3,6-trifluoro)phenethyl, (2,4,5-trifluoro)phenethyl, (2,4,6-trifluoro)phenethyl, (2,3,4-trifluoro)phenethyl, (3,4,5-trifluoro)phenethyl, (2,3,5-trifluoro)phenethyl, (2-chloro-5-fluoro)phenethyl, (3-fluoro-4-trifluoromethyl)phenethyl, (2-chloro-5-trifluoromethyl)phenethyl, (2-fluoro-3-chloro-5-trifluoromethyl)phenethyl, (2-fluoro-3-chloro)phenethyl, (4-fluoro-3-chloro)phenethyl, (2-fluoro-4-chloro)phenethyl, (2,3-difluoro-4-methyl)phenethyl, 2,6-difluoro-3-chlorophenethyl, (2,6-difluoro-3-methyl)phenethyl, (2-trifluoromethyl-5-chloro)phenethyl, (6-chloro-2-fluoro-5-methyl)phenethyl, (2,4-dichloro-5-fluoro)phenethyl, 5-chloro-2-fluorophenethyl, (2,5-difluoro-6-chloro)phenethyl, (2,3,4,5-tetrafluoro)phenethyl, (2-fluoro-4-trifluoromethyl)phenethyl, 2,3-(difluoro-4-trifluoromethyl)phenethyl, (2,5-di(trifluoromethyl))phenethyl, 2-fluoro-3,5-dibromo-phenethyl, (3-fluoro-4-nitro)phenethyl, (2-bromo-4-trifluoromethyl)phenethyl, 2-(bromo-5-fluoro)phenethyl, (2,6-difluoro-4-bromo)phenethyl, (2,6-difluoro-4-chloro)phenethyl, (3-chloro-5-fluoro)phenethyl, (2-bromo-5-trifluoromethyl)phenethyl and the like.

A specific embodiment of the invention relates to compounds in which at least one of the R¹, R², R³, R⁴, R^{I}, R^{II}, and R⁵ to R²⁹ radicals specified in the formula (I) and the formulae which follow is a C₁-C₃₀-alkyl radical in the form of a so-called swallowtail radical. The R¹, R², R³, R⁴, R^{I}, R^{II}, and R⁵ to R²⁹ radicals are then preferably a radical of the general formula (II)

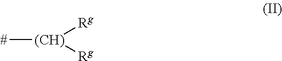

in which
# is a bonding site, and
the R^{g} radicals are selected from C₁- to C₂₈-alkyl, where the sum of the carbon atoms of the R^{g} radicals is an integer from 2 to 29.

In the formula (II), the R^{g} radicals are preferably selected from C₁- to C₁₂-alkyl, especially C₁- to C₈-alkyl.

Preferred radicals of the formula (II) are, for example:

1-ethylpropyl, 1-methylpropyl, 1-propylbutyl, 1-ethylbutyl, 1-methylbutyl, 1-butylpentyl, 1-propylpentyl, 1-ethylpentyl, 1-methylpentyl, 1-pentylhexyl, 1-butylhexyl, 1-propylhexyl, 1-ethylhexyl, 1-methylhexyl, 1-hexylheptyl, 1-pentylheptyl, 1-butylheptyl, 1-propylheptyl, 1-ethylheptyl, 1-methylheptyl, 1-heptyloctyl, 1-hexyloctyl, 1-pentyloctyl, 1-butyloctyl, 1-propyloctyl, 1-ethyloctyl, 1-methyloctyl, 1-octylnonyl, 1-heptylnonyl, 1-hexylnonyl, 1-pentylnonyl, 1-butylnonyl, 1-propylnonyl, 1-ethylnonyl, 1-methylnonyl, 1-nonyldecyl, 1-octyldecyl, 1-heptyldecyl, 1-hexyldecyl, 1-pentyldecyl, 1-butyldecyl, 1-propyldecyl, 1-ethyldecyl, 1-methyldecyl, 1-decylundecyl, 1-nonylundecyl, 1-octylundecyl, 1-heptylundecyl, 1-hexylundecyl, 1-pentylundecyl, 1-butylundecyl, 1-propylundecyl, 1-ethylundecyl, 1-methylundecyl, 1-undecyldodecyl, 1-decyldodecyl, 1-nonyldodecyl, 1-octyldodecyl, 1-heptyldodecyl, 1-hexyldodecyl, 1-pentyldodecyl, 1-butyldodecyl, 1-propyldodecyl, 1-ethyldodecyl, 1-methyldodecyl, 1-dodecyltridecyl, 1-undecyltridecyl, 1-decyltridecyl, 1-nonyltridecyl, 1-octyltridecyl, 1-heptyltridecyl, 1-hexyltridecyl, 1-pentyltridecyl, 1-butyltridecyl, 1-propyltridecyl, 1-ethyltridecyl, 1-methyltridecyl, 1-tridecyltetradecyl, 1-undecyltetradecyl, 1-decyltetradecyl, 1-nonyltetradecyl, 1-octyltetradecyl, 1-heptyltetradecyl, 1-hexyltetradecyl, 1-pentyltetradecyl, 1-butyltetradecyl, 1-propyltetradecyl, 1-ethyltetradecyl, 1-methyltetradecyl, 1-pentadecylhexadecyl, 1-tetradecylhexadecyl, 1-tridecylhexadecyl, 1-dodecylhexadecyl, 1-undecylhexadecyl, 1-decylhexadecyl, 1-nonylhexadecyl, 1-octylhexadecyl, 1-heptylhexadecyl, 1-hexylhexadecyl, 1-pentylhexadecyl, 1-butylhexadecyl, 1-propylhexadecyl, 1-ethylhexadecyl, 1-methylhexadecyl, 1-hexadecyloctadecyl, 1-pentadecyloctadecyl, 1-tetradecyloctadecyl, 1-tridecyloctadecyl, 1-dodecyloctadecyl, 1-undecyloctadecyl, 1-decyloctadecyl, 1-nonyloctadecyl, 1-octyloctadecyl, 1-heptyloctadecyl, 1-hexyloctadecyl, 1-pentyloctadecyl, 1-butyloctadecyl, 1-propyloctadecyl, 1-ethyloctadecyl, 1-methyloctadecyl, 1-nonadecyleicosanyl, 1-octadecyleicosanyl, 1-heptadecyleicosanyl, 1-hexadecyleicosanyl, 1-pentadecyleicosanyl, 1-tetradecyleicosanyl, 1-tridecyleicosanyl, 1-dodecyleicosanyl, 1-undecyleicosanyl, 1-decyleicosanyl, 1-nonyleicosanyl, 1-octyleicosanyl, 1-heptyleicosanyl, 1-hexyleicosanyl, 1-pentyleicosanyl, 1-butyleicosanyl, 1-propyleicosanyl, 1-ethyleicosanyl, 1-methyleicosanyl, 1-eicosanyldocosanyl, 1-nonadecyldocosanyl, 1-octadecyldocosanyl, 1-heptadecyldocosanyl, 1-hexadecyldocosanyl, 1-pentadecyldocosanyl, 1-tetradecyldocosanyl, 1-tridecyldocosanyl, 1-undecyldocosanyl, 1-decyldocosanyl, 1-nonyldocosanyl, 1-octyldocosanyl, 1-heptyldocosanyl, 1-hexyldocosanyl, 1-pentyldocosanyl, 1-butyldocosanyl, 1-propyldocosanyl, 1-ethyldocosanyl, 1-methyldocosanyl, 1-tricosanyltetracosanyl, 1-docosanyltetracosanyl, 1-nonadecyltetracosanyl, 1-octadecyltetracosanyl, 1-heptadecyltetracosanyl, 1-hexadecyltetracosanyl, 1-pentadecyltetracosanyl, 1-pentadecyltetracosanyl, 1-tetradecyltetracosanyl, 1-tridecyltetracosanyl, 1-dodecyltetracosanyl, 1-undecyltetracosanyl, 1-decyltetracosanyl, 1-nonyltetracosanyl, 1-octyltetracosanyl, 1-heptyltetracosanyl, 1-hexyltetracosanyl, 1-pentyltetracosanyl, 1-butyltetracosanyl, 1-propyltetracosanyl, 1-ethyltetracosanyl, 1-methyltetracosanyl, 1-heptacosanyloctacosanyl, 1-hexacosanyloctacosanyl, 1-pentacosanyloctacosanyl, 1-tetracosanyloctacosanyl, 1-tricosanyloctacosanyl, 1-docosanyloctacosanyl, 1-nonadecyloctacosanyl, 1-octadecyloctacosanyl, 1-heptadecyloctacosanyl, 1-hexadecyloctacosanyl, 1-hexadecyloctacosanyl, 1-pentadecyloctacosanyl, 1-tetradecyloctacosanyl, 1-tridecyloctacosanyl, 1-dodecyloctacosanyl, 1-undecyloctacosanyl, 1-decyloctacosanyl, 1-nonyloctacosanyl, 1-octyloctacosanyl, 1-heptyloctacosanyl, 1-hexyloctacosanyl, 1-pentyloctacosanyl, 1-butyloctacosanyl, 1-propyloctacosanyl, 1-ethyloctacosanyl, 1-methyloctacosanyl.

### R¹ and R⁴ Radicals:

In the compounds of the general formula (I), the R¹ and R⁴ radicals may both have the same or a different definition. R¹ and R⁴ preferably have the same definition.

Preferably R¹ and R⁴ are each independently selected are from hydrogen, unsubstituted alkyl, aralkyl, unsubstituted aryl, alkaryl and halogen-substituted aryl. R¹ and R⁴ may also be selected from thiophenyl(thienyl) and oligothiophenyl where the two last-mentioned radicals may carry one or more C₁-C₃₀ alkyl radicals, in particular C₁-C₁₂ alkyl.

In a specific embodiment, R¹ and/or R⁴ are each unsubstituted or substituted C₁- to C₃₀-alkyl. Preferably, R¹ and/or R⁴ are each unsubstituted or substituted C₁- to C₁₂-alkyl. More preferably, R¹ and/or R⁴ are each unsubstituted linear C₁- to C₁₂-alkyl, especially unsubstituted linear C₄- to C₁₂-alkyl, such as n-octyl, n-nonyl, n-decyl, n-undecyl, n-dodecyl. R¹ and R⁴ are especially each unsubstituted or substituted C₁- to C₁₂-alkyl or unsubstituted or substituted C₁- to C₁₂-alkoxy, more especially unsubstituted linear C₁- to C₁₂-alkyl, even more especially unsubstituted linear C₄- to C₁₂-alkyl, such as n-octyl, n-nonyl, n-decyl, n-undecyl, n-dodecyl.

In a further specific embodiment, R¹ and/or R⁴ are each a radical of the general formula (II)

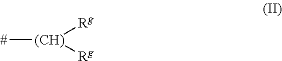

in which
# represents a bonding site, and
the R^{g} radicals are selected from C₁- to C₂₈-alkyl, where the sum of the carbon atoms of the R^{d} radicals is an integer from 2 to 29.

In the case that R¹ and/or R⁴ is a radical of the general formula (II), reference is made completely to the suitable and preferred R^{g} radicals specified above.

When R¹ and/or R⁴ are each substituted alkyl, the alkyl radicals, depending on their chain length, have preferably 1, 2, 3, 4, 5, 6, 7, 8, 9, 10 or more than 10 substituents. The substituents of the alkyl radicals are preferably selected independently from aryl, fluorine, chlorine and nitro.

In a further specific embodiment, R¹ and/or R⁴ are each thienyl which may be unsubstituted or may carry one or more, e.g. 1, 2 or 3, C₁-C₁₂-alkyl radicals. In a further specific embodiment, R¹ and/or R⁴ are each oligothiophenyl where each thiophene unit may be unsubstituted or may carry one or more, e.g. 1 or 2, C₁-C₁₂-alkyl radicals.

In a further specific embodiment, R¹ and/or R⁴ are each alkaryl or aryl, where aryl and the aryl moiety of alkaryl is preferably phenyl, naphthyl, anthracyl or phenanthryl.

R¹ and R⁴ are preferably each selected from hydrogen and groups of the general formulae (III.1) to (III.12)

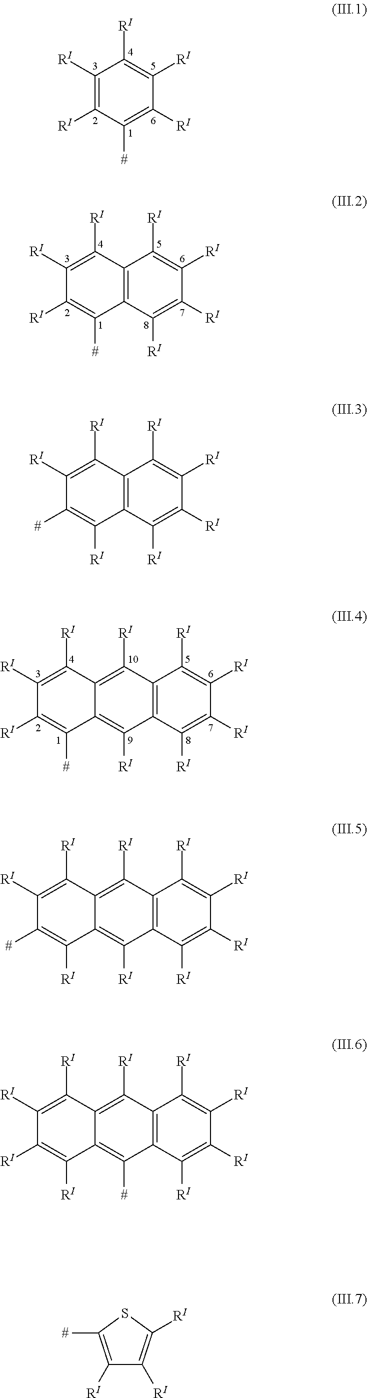

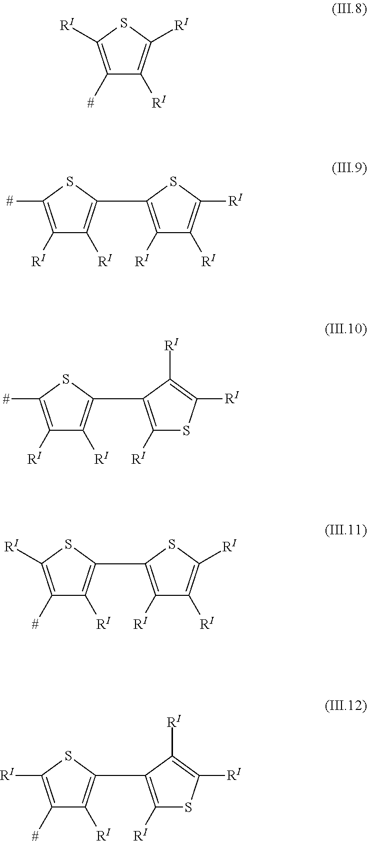

in which
# is the bonding site to the benzene ring, and
R^{I}, are each independently selected from hydrogen, fluorine, chlorine, unsubstituted alkyl and substituted alkyl.

(The numbering of the aromatic ring systems is used hereinafter to specify the position of the substituents.)

Preferably, 0, 1 or 2 of the R^{I} radicals in the groups of the formula (III.1) have a definition other than hydrogen. Monosubstituted groups of the formula (III.1) preferably have an R^{I} radical in the 4 position. Disubstituted groups of the formula (III.1) preferably have two R^{I} radicals in the 3 position and in the 5 position.

Preferably, 0, 1, 2, 3 or 4 of the R^{I} radicals in the groups of the formula (III.2) have a definition other than hydrogen. More preferably, 0 or 1 of the R^{I} radicals in the groups of the formula (III.2) have a definition other than hydrogen. Monosubstituted groups of the formula (III.2) preferably have an R^{I} radical in the 4 position.

Preferably, 0, 1, 2, 3 or 4 of the R^{I} radicals in the groups of the formula (III.3) have a definition other than hydrogen. More preferably, 0 or 1 of the R^{I} radicals in the groups of the formula (III.3) have a definition other than hydrogen.

Preferably, 0, 1, 2, 3, 4, 5 or 6 of the R^{I} radicals in the groups of the formula (III.4) have a definition other than hydrogen. More preferably, 0 or 1 of the R^{I} radicals in the groups of the formula (III.4) have a definition other than hydrogen.

Preferably, 0, 1, 2, 3, 4, 5 or 6 of the R^{I} radicals in the groups of the formula (III.5) have a definition other than hydrogen. More preferably, 0 or 1 of the R^{I} radicals in the groups of the formula (III.5) have a definition other than hydrogen.

Preferably, 0, 1, 2, 3, 4, 5 or 6 of the R^{I} radicals in the groups of the formula (III.6) have a definition other than hydrogen. More preferably, 0 or 1 of the R^{I} radicals in the groups of the formula (III.6) have a definition other than hydrogen.

Preferably, 0, 1 or 2 of the R^{I} radicals in the groups of the formula (III.7) have a definition other than hydrogen. More preferably, 0 or 1 of the R^{I} radicals in the groups of the formula (III.7) have a definition other than hydrogen.

Preferably, 0, 1 or 2 of the R^{I} radicals in the groups of the formula (III.8) have a definition other than hydrogen. More preferably, 0 or 1 of the R^{I} radicals in the groups of the formula (III.8) have a definition other than hydrogen.

Preferably, 0, 1, 2, 3 or 4 of the R^{I} radicals in the groups of the formulae (III.9) to (III.12) have a definition other than hydrogen. Preferably, 0, 1 or 2 of the R^{I} radicals on each thiophene ring have a definition other than hydrogen. More preferably, 0 or 1 of the R^{I} radicals in the groups of the formulae (III.9) to (III.12) have a definition other than hydrogen.

Preferably, the R^{I} radicals in the (III.1) to (III.12) groups are each independently selected from hydrogen, C₁-C₂₀-alkyl, fluorine and chlorine.

More preferably, the R^{I} radicals in the (III.1) to (III.12) groups are each independently selected from hydrogen and C₁- to C₁₂-alkyl.

In a specific embodiment, the R^{I} radicals which have a definition other than hydrogen in the (III.1) to (III.12) groups are selected from unsubstituted C₁- to C₄-alkyl groups, such as methyl, ethyl, n-propyl, isopropyl, n-butyl and tert-butyl.

In a further specific embodiment, the R^{I} radicals which have a definition other than hydrogen in the (III.1) to (III.12) groups are selected from unsubstituted linear C₄- to C₁₂-alkyl groups, especially unsubstituted linear C₈-C₁₂-alkyl, such as n-octyl, n-nonyl, n-decyl, n-undecyl, n-dodecyl.

In a further specific embodiment, the R^{I} radicals which have a definition other than hydrogen in the (III.1) to (III.12) groups are selected from radicals of the general formula (II)

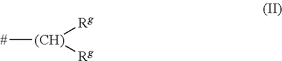

in which
# represents a bonding site, and
the R^{g} radicals are selected from C₁- to C₂₈-alkyl, where the sum of the carbon atoms of the R^{g} radicals is an integer from 2 to 29.

In the case that R^{I} is a radical of the general formula (II), reference is made completely to the suitable and preferred R^{g} radicals specified above.

In particular, R¹ and R⁴ may be independently of each other selected from phenyl, naphthyl, anthracyl, thiophen-2-yl, thiopheny-3-yl and oligothiophenyl, where phenyl, naphthyl, anthracyl, thiophen-2-yl and thiopheny-3-yl may be unsubstituted or may carry one or two C₁-C₁₂ alkyl radicals and where oligothiophenyl may carry one or two C₁-C₁₂ alkyl radical on each thiophene unit. A special embodiment relates to compounds of the formula I, wherein R¹ and/or R⁴ are thiophen-2-yl, thiopheny-3-yl or oligothiophenyl, where thiophen-2-yl and thiopheny-3-yl may be unsubstituted or may carry one C₁-C₁₂ alkyl radical and where oligothiophenyl may carry one C₁-C₁₂ alkyl radical on each thiophene unit.

More particularly, R¹ and R⁴ are each independently selected from hydrogen and groups of the general formulae (III.1a), (III.1b), (III.1c), (III.1d), (III.2a), (III.4a), (III.7a), III.7b), (III.8a), (III.8b), (III.9a), (III.9b), (III.9c), (III.9d), (III.10a), (III.10b), (III.10c), (III.10d), (III.11a), (III.11b), (III.11c), (III.11d), (III.12a), (III.12b), (III.12c) and (III.12d):

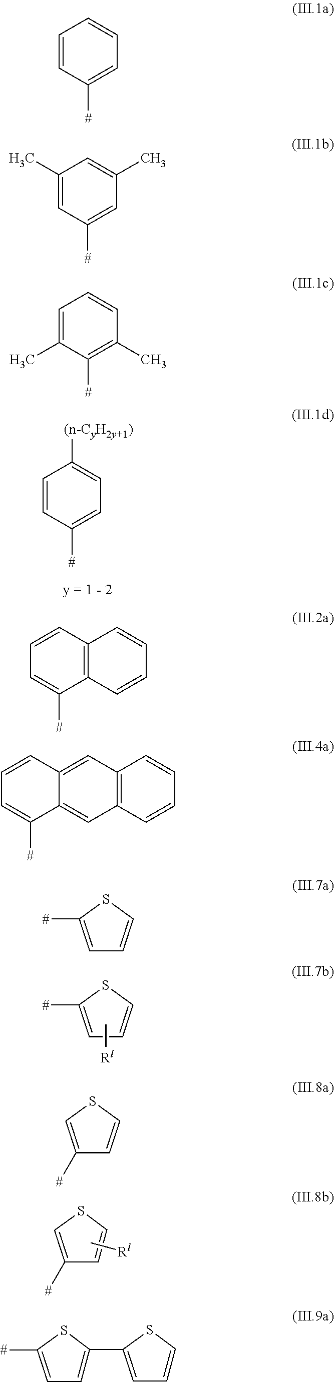

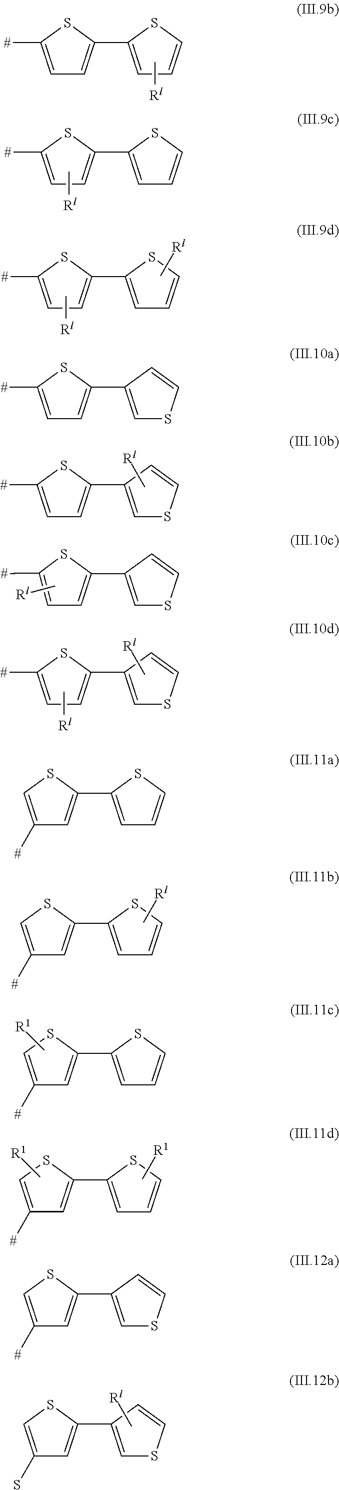

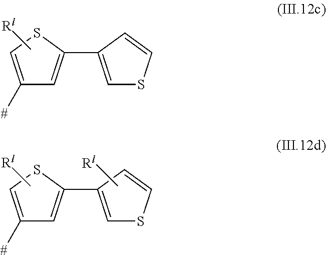

in which
# is the bonding site to the benzene ring and
R¹ is C₁-C₈-alkyl.

In particular, the R¹ and R⁴ groups are each independently selected from hydrogen and phenyl. Likewise in particular, the R¹ and R⁴ groups are each independently selected from hydrogen, phenyl, thiophen-2-yl, thiophen-3-yl and 5-methylthiophen-2-yl. R¹ and R⁴ are especially both hydrogen or are both phenyl. Likewise, R¹ and R⁴ are especially both thiophen-2-yl or both 5-methylthiophen-2-yl.

### R² and R³ Radicals

In the compounds of the general formula (I), the R² and R³ radicals may both have the same definition or each have a different definition. R² and R³ preferably have the same definition.

R² and R³ are preferably each independently selected from hydrogen, unsubstituted alkyl, aralkyl, unsubstituted aryl, alkaryl and halogen-substituted aryl. R² and R³ may also be selected from thiophenyl and oligothiophenyl where the two last-mentioned radicals may carry one or more C₁-C₃₀ alkyl, in particular C₁-C₁₂ alkyl, radicals.

In a specific embodiment, R² and/or R³ are each unsubstituted or substituted C₁- to C₃₀-alkyl. R² and/or R³ are preferably each unsubstituted or substituted C₁- to C₁₂-alkyl. R² and/or R³ are more preferably each unsubstituted linear C₁- to C₁₂-alkyl, especially unsubstituted linear C₄- to C₁₂-alkyl, such as n-octyl, n-nonyl, n-decyl, n-undecyl, n-dodecyl. R² and R³ are especially each unsubstituted or substituted C₁- to C₁₂-alkyl or unsubstituted or substituted C₁- to C₁₂-alkoxy, more especially unsubstituted linear C₁- to C₁₂-alkyl, even more especially unsubstituted linear C₄- to C₁₂-alkyl, such as n-octyl, n-nonyl, n-decyl, n-undecyl, n-dodecyl.

In a further specific embodiment, R² and/or R³ are each a radical of the general formula (II)

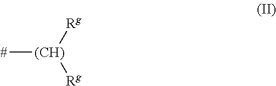

in which
# is a bonding site, and
the R^{g} radicals are selected from C₁- to C₂₈-alkyl, where the sum of the carbon atoms of the R^{g} radicals is an integer from 2 to 29.

In the case that R² and/or R³ is a radical of the general formula (II), full reference is made to the aforementioned suitable and preferred R^{g} radicals.

When the R² and/or R³ groups in the compounds of the general formula (I) are substituted alkyl, the alkyl radicals, depending on their chain length, have preferably 1, 2, 3, 4, 5, 6, 7, 8, 9, 10 or more than 10 substituents. The substituents of the alkyl radicals are preferably each independently selected from aryl, fluorine, chlorine and nitro.

In a further specific embodiment, R² and/or R³ are selected from thienyl which may be unsubstituted or may carry one or more, e.g. 1, 2 or 3, C₁-C₁₂-alkyl radicals. In a further specific embodiment, R² and/or R³ are selected from oligothiophenyl where each thiophene unit may be unsubstituted or may carry one or more, e.g. 1 or 2, C₁-C₁₂-alkyl radicals.

In a further specific embodiment, R² and/or R³ are selected from alkaryl and aryl, where aryl and the aryl moiety of alkaryl is preferably phenyl, naphthyl, anthracyl or phenanthryl.

In a specific embodiment, the R² and R³ groups in the compounds of the general formula (I) are each independently selected from hydrogen and groups of the general formulae (IV.1) to (IV.12)

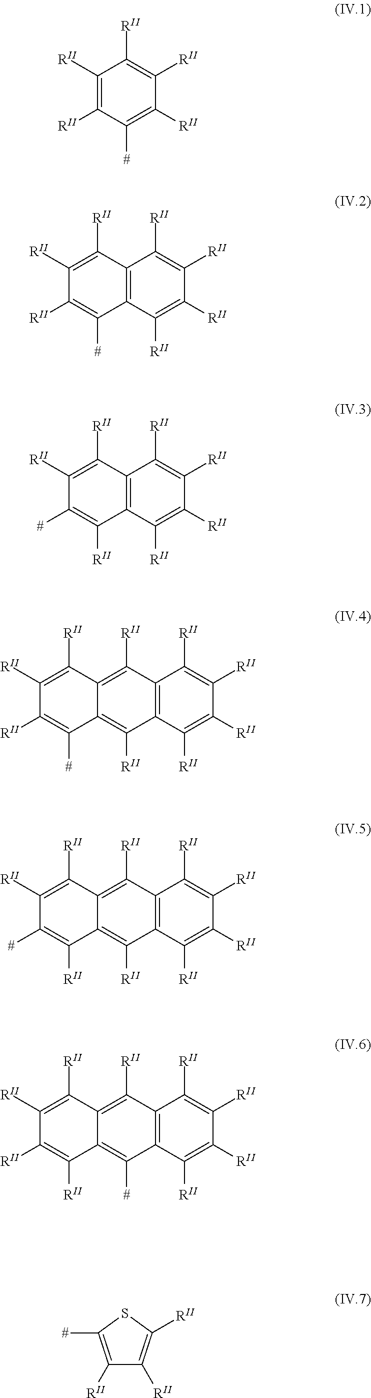

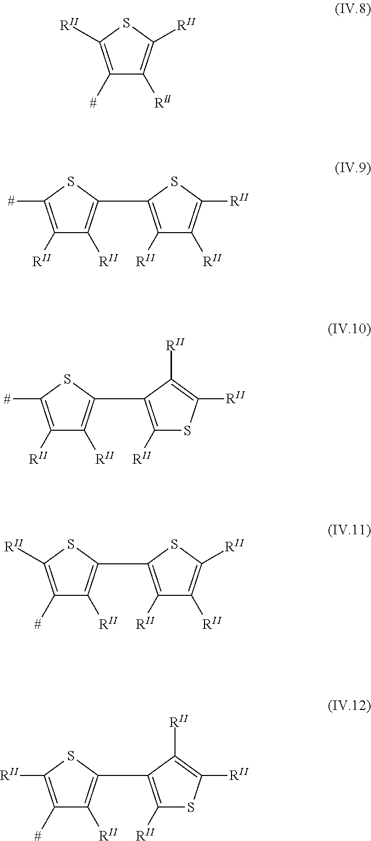

in which
# is the bonding site to the benzene ring, and
R^{II} are each independently selected from hydrogen, fluorine, chlorine, unsubstituted alkyl and substituted alkyl.

Preferably, 0, 1 or 2 of the R^{II} radicals in the groups of the formula (IV.1) have a definition other than hydrogen. Monosubstituted groups of the formula (IV.1) preferably have an R^{II} radical in the 4 position. Disubstituted groups of the formula (IV.1) preferably have two R^{II} radicals in the 3 position and in the 5 position.

Preferably, 0, 1, 2, 3 or 4 of the R^{II} radicals in the groups of the formula (IV.2) have a definition other than hydrogen. More preferably, 0 or 1 of the R^{II} radicals in the groups of the formula (IV.2) have a definition other than hydrogen. Monosubstituted groups of the formula (IV.2) preferably have an R^{II} radical in the 4 position.

Preferably, 0, 1, 2, 3 or 4 of the R^{II} radicals in the groups of the formula (IV.3) have a definition other than hydrogen. More preferably, 0 or 1 of the R^{II} radicals in the groups of the formula (IV.3) have a definition other than hydrogen.

Preferably, 0, 1, 2, 3, 4, 5 or 6 of the R^{II} radicals in the groups of the formula (IV.4) have a definition other than hydrogen. More preferably, 0 or 1 of the R^{II} radicals in the groups of the formula (IV.4) have a definition other than hydrogen.

Preferably, 0, 1, 2, 3, 4, 5 or 6 of the R^{II} radicals in the groups of the formula (IV.5) have a definition other than hydrogen. More preferably, 0 or 1 of the R^{II} radicals in the groups of the formula (IV.5) have a definition other than hydrogen.

Preferably, 0, 1, 2, 3, 4, 5 or 6 of the R^{II} radicals in the groups of the formula (IV.6) have a definition other than hydrogen. More preferably, 0 or 1 of the R^{II} radicals in the groups of the formula (IV.6) have a definition other than hydrogen.

Preferably, 0, 1 or 2 of the R^{II} radicals in the groups of the formula (IV.7) have a definition other than hydrogen. More preferably, 0 or 1 of the R^{II} radicals in the groups of the formula (IV.7) have a definition other than hydrogen.

Preferably, 0, 1 or 2 of the R^{II} radicals in the groups of the formula (IV.8) have a definition other than hydrogen. More preferably, 0 or 1 of the R^{II} radicals in the groups of the formula (IV.8) have a definition other than hydrogen.

Preferably, 0, 1, 2, 3 or 4 of the R^{II} radicals in the groups of the formulae (IV.9) to (IV.12) have a definition other than hydrogen. Preferably, 0, 1 or 2 of the R^{II} radicals on each thiophene ring have a definition other than hydrogen. More preferably, 0 or 1 of the R^{II} radicals in the groups of the formulae (IV.9) to (IV.12) have a definition other than hydrogen.

In a specific embodiment, the R^{II} radicals which have a definition other than hydrogen in the (IV.1) to (IV.12) groups are selected from unsubstituted C₁- to C₄-alkyl groups, such as methyl, ethyl, n-propyl, isopropyl, n-butyl and tert-butyl.

In a further specific embodiment, the R^{II} radicals which have a definition other than hydrogen in the (IV.1) to (IV.12) groups are selected from unsubstituted linear C₄- to C₁₂-alkyl groups, especially unsubstituted linear C₈-C₁₂-alkyl such as n-octyl, n-nonyl, n-decyl, n-undecyl, n-dodecyl.

In a further specific embodiment, the R^{II} radicals which have a definition other than hydrogen in the (IV.1) to (IV.12) groups are selected from radicals of the general formula (II)

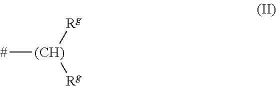

in which
# represents a bonding site, and
the R^{g} radicals are selected from C₁- to C₂₈-alkyl, where the sum of the carbon atoms of the R^{g} radicals is an integer from 2 to 29.

In the case that R^{II} is a radical of the general formula (II), reference is made completely to the suitable and preferred R^{g} radicals specified above.

In particular, R² and R³ may be independently of each other selected from phenyl, naphthyl, anthracyl, thiophen-2-yl, thiopheny-3-yl and oligothiophenyl, where phenyl, naphthyl, anthracyl, thiophen-2-yl and thiopheny-3-yl may be unsubstituted or may carry one or two C₁-C₁₂ alkyl radicals and where oligothiophenyl may carry one C₁-C₁₂ alkyl radical on each thiophene unit. A special embodiment relates to compounds of the formula I, wherein R² and/or R³ are thiophen-2-yl, thiopheny-3-yl or oligothiophenyl, where thiophen-2-yl and thiopheny-3-yl may be unsubstituted or may carry one C₁-C₁₂ alkyl radical and where oligothiophenyl may carry one C₁-C₁₂ alkyl radical on each thiophene unit.

More particularly, R² and R³ are each independently selected from hydrogen and groups of the general formulae (IV.1a), (IV.1b), (IV.1c), (IV.1d), (IV.2a), (IV.4a), (IV.7a), (IV.7b), (IV.8a), (IV.8b), (IV.9a), (IV.9b), (IV.9c), (IV.9d), (IV.10a), (IV.10b), (IV.10c), (IV.10d), (IV.11a), (IV.11b), (IV.11c), (IV.11d), (IV.12a), (IV.12b), (IV.12c) and (IV.11d):

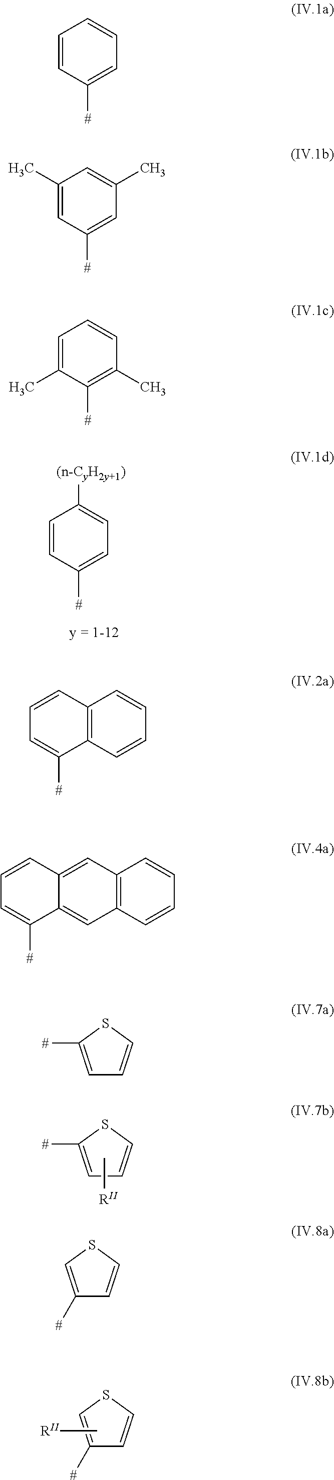

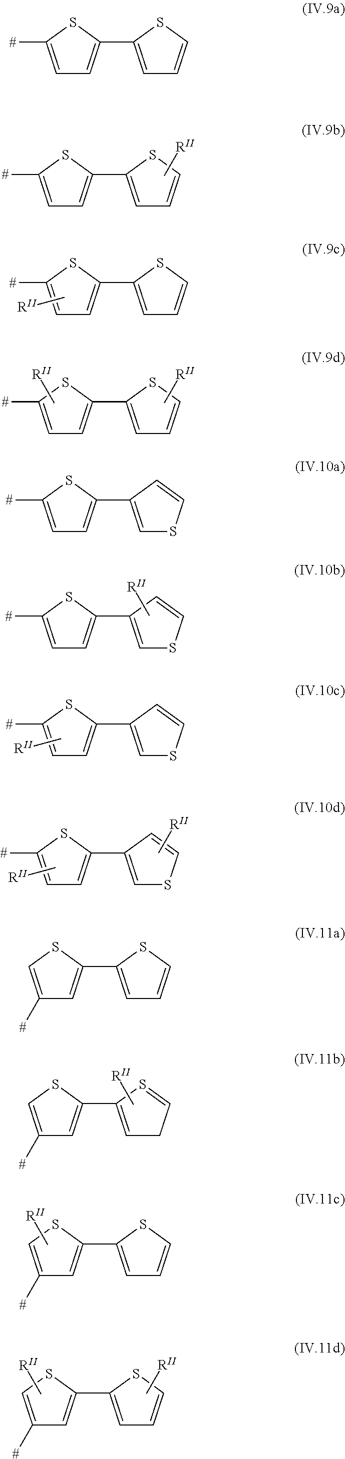

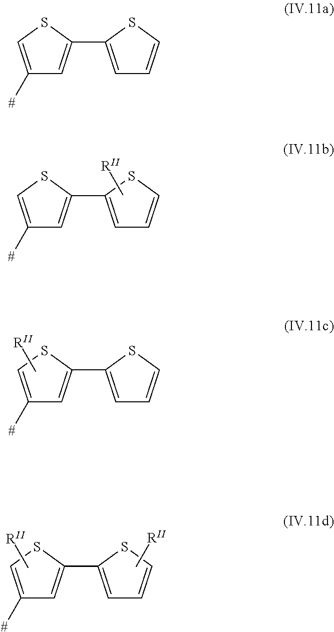

in which
# is the bonding site to the benzene ring; and
R^{II} is C₁-C₈-alkyl.

The R² and R³ groups are especially each independently selected from hydrogen and phenyl. R² and R³ are especially both hydrogen or both phenyl.

### Fused R² and R³ Radicals

In a specific embodiment, the two R² and R³ radicals in the compounds of the general formula (I), together with the carbon atoms of the benzene ring to which they are bonded, are a fused ring system having 1, 2, 3 or 4 further rings.

Preferably, R² and R³ in the compounds of the general formula (I) together are a group which is selected from groups of the general formulae (V.1), (V.2) and (V.3)

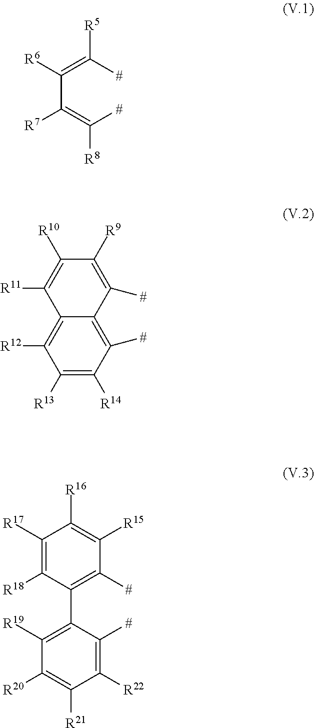

in which
# in each case is the bonding site to the benzene ring;
R⁵, R⁶, R⁷ and R⁸ are each independently hydrogen, C₁-C₂₀-alkyl, halogen or phenyl, where phenyl may be unsubstituted or may bear 1, 2 or 3 R^{a} radicals;
R⁹, R¹⁰, R¹¹, R¹², R¹³ and R¹⁴ are each independently hydrogen, C₁-C₂₀-alkyl, halogen or phenyl, where phenyl may be unsubstituted or may bear 1, 2 or 3 R^{a} radicals; and
R¹⁵, R¹⁶, R¹⁷, R¹⁸, R¹⁹, R²⁰, R²¹ and R²² are each independently hydrogen, C₁-C₂₀-alkyl, halogen or phenyl, where phenyl may be unsubstituted or may bear 1, 2 or 3 R^{a} radicals, in which R^{a} is C₁-C₁₀-alkyl or C₁-C₆-alkoxy.

More preferably, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R^{a} independently of one another and preferably in combination preferably have the meanings given below:
R⁵, R⁶, R⁷ and R⁸ are each independently hydrogen, C₁-C₂₀-alkyl, or phenyl, where phenyl may be unsubstituted or may bear 1, 2 or 3 R^{a} radicals;
R⁹, R¹⁰, R¹¹, R¹², R¹³ and R¹⁴ are each independently hydrogen, C₁-C₂₀-alkyl, or phenyl, where phenyl may be unsubstituted or may bear 1, 2 or 3 R^{a} radicals; and
R¹⁵, R¹⁶, R¹⁷, R¹⁸, R¹⁹, R²⁰, R²¹ and R²² are each independently hydrogen, C₁-C₂₀-alkyl, or phenyl, where phenyl may be unsubstituted or may bear 1, 2 or 3 R^{a} radicals, in which
R^{a} is C₁-C₁₀-alkyl.

In a preferred embodiment, R² and R³ together are a group of the formula (V.1). Preferably, R⁵, R⁶, R⁷ and R⁸ in the groups (V.1) are each hydrogen.

Preferably, 0, 1, 2, 3 or 4 of the R³ radicals in the groups of the formulae (V.2) and (V.3) have a definition other than hydrogen.

Preferably, the R⁵ to R²² radicals in the (V.2) and (V.3) groups are each independently selected from hydrogen, C₁- to C₂₀-alkyl, fluorine, chlorine and phenyl.

More preferably, the R⁵ to R²² radicals in the (V.1) to (V.3) groups are each independently selected from hydrogen, C₁- to C₁₂-alkyl, fluorine, chlorine and phenyl. Even more preferably, the R⁵ to R²² radicals in the (V.1) to (V.3) groups are each independently selected from hydrogen, C₁- to C₁₂-alkyl and phenyl.

In a specific embodiment, the R⁵ to R²² radicals which have a definition other than hydrogen in the (V.1) to (V.3) groups are selected from unsubstituted C₁- to C₄-alkyl groups, such as methyl, ethyl, n-propyl, isopropyl, n-butyl and tert-butyl.

In a further specific embodiment, the R⁵ to R²² radicals which have a definition other than hydrogen in the (V.1) to (V.3) groups are selected from unsubstituted linear C₄- to C₁₂-alkyl groups, such as n-octyl, n-nonyl, n-decyl, n-undecyl, n-dodecyl.

In a further specific embodiment, the R⁵ to R²² radicals which have a definition other than hydrogen in the (V.1) to (V.3) groups are selected from radicals of the general formula (II)

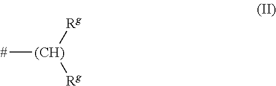

in which
# represents a bonding site, and
the R^{g} radicals are selected from C₁- to C₂₈-alkyl, where the sum of the carbon atoms of the R^{g} radicals is an integer from 2 to 29.

In the case that R⁵ to R²² is a radical of the general formula (II), reference is made completely to the suitable and preferred R^{g} radicals specified above.

More preferably, R² and R³ together are a (V.1), (V.2) or (V.3) group in which the R⁵ to R²² radicals are all hydrogen.

### Fused A Groups

Preferably, the A groups in the compounds of the general formula (I) are selected from groups of the general formulae (VI.1), (VI.2), (VI.3), (VI.4) and (VI.5)

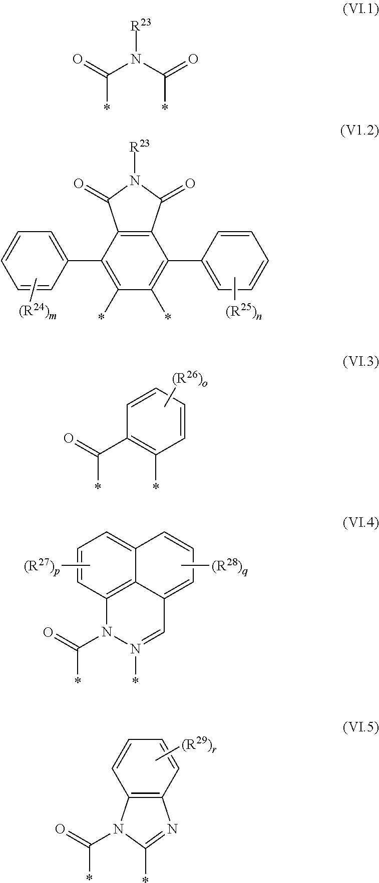

* in each case is the bonding site to the perylene base skeleton,
R²³ is hydrogen, and in each case unsubstituted or substituted alkyl, cycloalkyl, heterocyclyl, aryl or hetaryl,
R²⁴, R²⁵ are each independently halogen, unsubstituted alkyl, substituted alkyl, unsubstituted aryl or substituted aryl, and where two R²⁴ and/or R²⁵ radicals bonded to adjacent carbon atoms may also be a fused-on benzene ring,
R²⁶ is halogen, unsubstituted alkyl, substituted alkyl, unsubstituted aryl or substituted aryl, and where two R²⁶ radicals bonded to adjacent carbon atoms may be a fused-on benzene ring,
R²⁷, R²⁸ are each independently halogen, unsubstituted alkyl, substituted alkyl, unsubstituted aryl or substituted aryl, and where two R²⁷ and/or R²⁸ radicals bonded to adjacent carbon atoms may also be a fused-on benzene ring,
R²⁹ is halogen, unsubstituted alkyl, substituted alkyl, unsubstituted aryl or substituted aryl, and where two R²⁹ radicals bonded to adjacent carbon atoms may be a fused-on benzene ring,
m, n are each independently 0, 1, 2 or 3,
o, p, q, r are each 0, 1 or 2.

The A group is more preferably selected from groups of the general formulae (VI.1), (VI.2), (VI.3), (VI.4) and (VI.5) in which
* in each case is the bonding site to the perylene base skeleton,
R²³ is hydrogen, C₁-C₂₀-alkyl, C₃-C₇-cycloalkyl, C₃-C₇-heterocyclyl, C₆-C₁₄-aryl-C₁-C₂₀-alkyl, C₆-C₁₄-aryl, C₁-C₂₀-alkyl-C₆-C₁₄-aryl, or a 5- or 6-membered heteroaromatic radical which has 1, 2 or 3 heteroatoms selected from O, S and N as ring members, and in which the heteroaromatic radical may have a fused-on benzene ring or naphthalene ring and may have 1, 2, 3, 4, 5 or 6 R^{b} substituents, and the aryl group in C₆-C₁₄-aryl-C₁-C₂₀-alkyl, C₆-C₁₄-aryl, C₁-C₂₀-alkyl-C₆-C₁₄-aryl may have 1, 2, 3, 4, 5 or 6 R^{b} substituents,
R²⁴, R²⁵ are each independently C₁-C₂₀-alkyl, halogen or phenyl, where phenyl may be unsubstituted or may bear 1, 2 or 3 R^{c} radicals, and where two R²⁴ and/or R²⁵ radicals bonded to adjacent carbon atoms may also be a fused-on benzene ring,
R²⁶ is C₁-C₂₀-alkyl, halogen or phenyl, where phenyl may be unsubstituted or may bear 1, 2 or 3 R^{c} radicals, and where two R²⁶ radicals bonded to adjacent carbon atoms may be a fused-on benzene ring,
R²¹, R²⁸ are each independently C₁-C₂₀-alkyl, halogen or phenyl, where phenyl may be unsubstituted or may bear 1, 2 or 3 R^{c} radicals, and where two R²¹ and/or R²⁸ radicals bonded to adjacent carbon atoms may also be a fused-on benzene ring,
R²⁹ is C₁-C₂₀-alkyl, halogen or phenyl, where phenyl may be unsubstituted or may bear 1, 2 or 3 R^{c} radicals, and where two R²⁹ radicals bonded to adjacent carbon atoms may be a fused-on benzene ring,
m, n are each independently 0, 1, 2 or 3,
o, p, q, r are each 0, 1 or 2,
R^{b} is fluorine, chlorine, cyano, nitro or C₁-C₂₀-alkoxy, and
R^{c} is fluorine, chlorine, cyano, nitro, C₁-C₂₀-alkyl or C₁-C₂₀-alkoxy.

A is more preferably a group of the formula (VI.1) in which R²³ is phenyl, C₁-C₆-alkyl or hydrogen.

A specific embodiment is a perylene compound of the formula (I) in which
R¹ and R⁴ are both phenyl,
R² and R³ together are a radical of the formula (V.1) in which R⁵, R⁶, R⁷ and R⁸ are each hydrogen, and
A is a radical of the formula (VI.1) in which R²³ is phenyl or hydrogen.

A further specific embodiment is a perylene compound of the formula (I) in which
R¹ and R⁴ are both phenyl;
R² and R³ together are a radical of the formula (V.1) in which R⁵, R⁶, R⁷ and R⁸ are each hydrogen or R² and R³ together are a radical of the formula (V.2) in which R⁹, R¹⁰, R¹¹, R¹², R¹³ and R¹⁴ are each hydrogen; and
A is a radical of the formula (VI.1) in which R²³ is C₁-C₄-alkyl.

A further specific embodiment is a perylene compound of the formula (I) in which
R¹ and R⁴ are both phenyl;
R² and R³ together are a radical of the formula (V.1) in which R⁵, R⁶, R⁷, and R⁸ are each hydrogen or R² and R³ together are a radical of the formula (V.2) in which R⁹, R¹⁰, R¹¹, R¹², R¹³ and R¹⁴ are each hydrogen; and
A is a radical of the formula (VI.4) in which p is 0 and q is 0.

A further specific embodiment is a perylene compound of the formula (I) in which
R¹ and R⁴ are both phenyl;
R² and R³ together are a radical of the formula (V.1) in which R⁵, R⁶, R⁷ and R⁸ are each hydrogen or R² and R³ together are a radical of the formula (V.2) in which R⁹, R¹⁰, R¹¹, R¹², R¹³ and R¹⁴ are each hydrogen; and
A is a radical of the formula (VI.5) in which r is 0.

Examples of substituted perylenes which are preferentially suitable for use in organic solar cells are reproduced hereinafter.

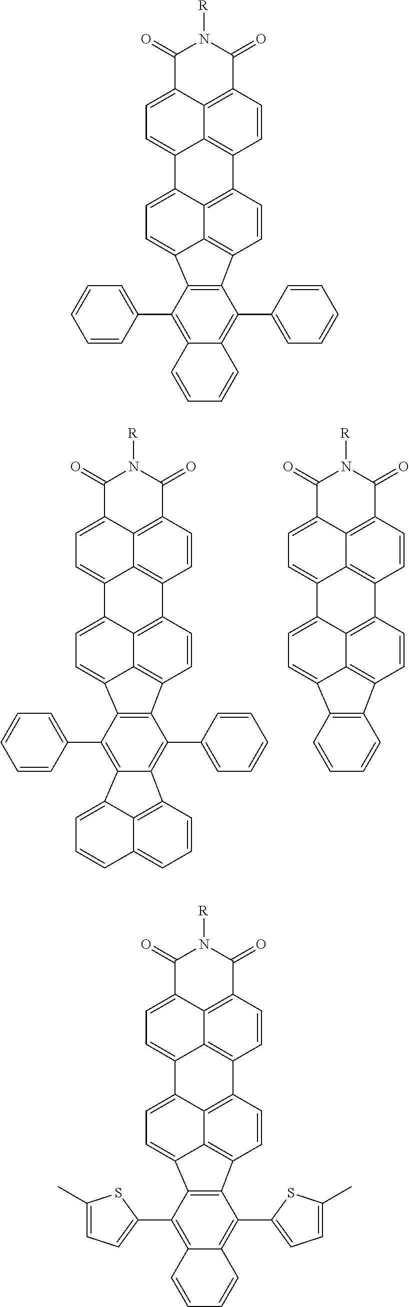

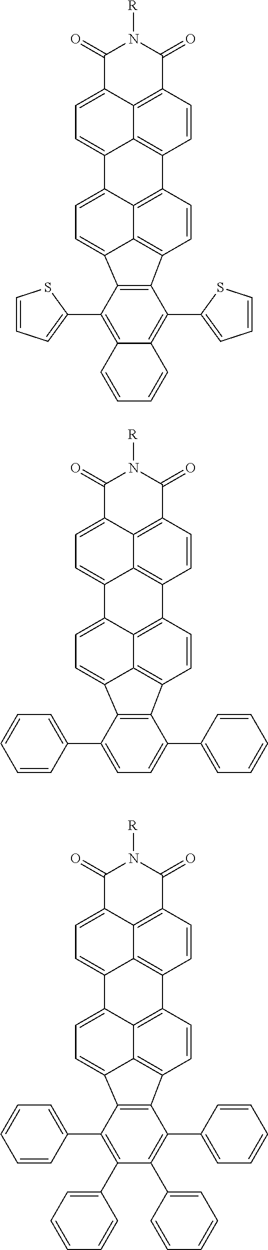

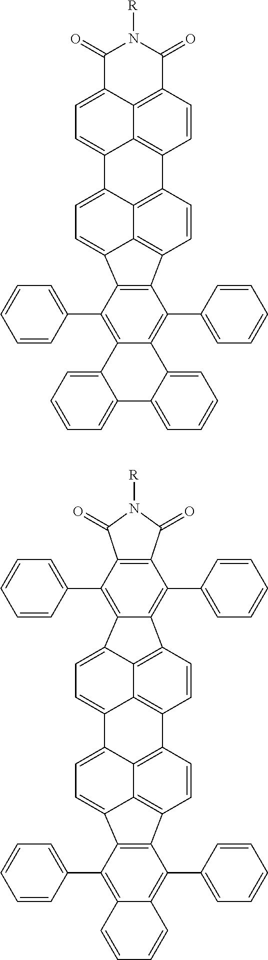

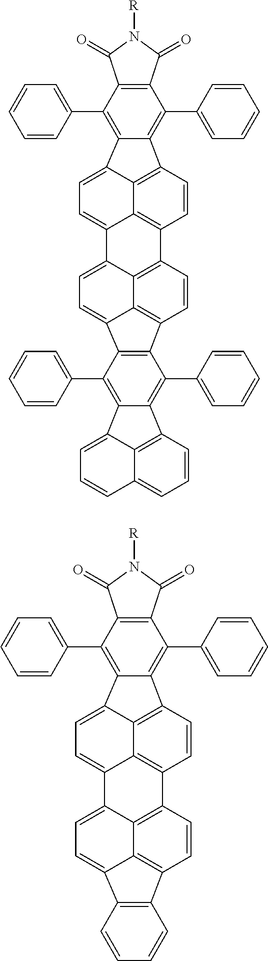

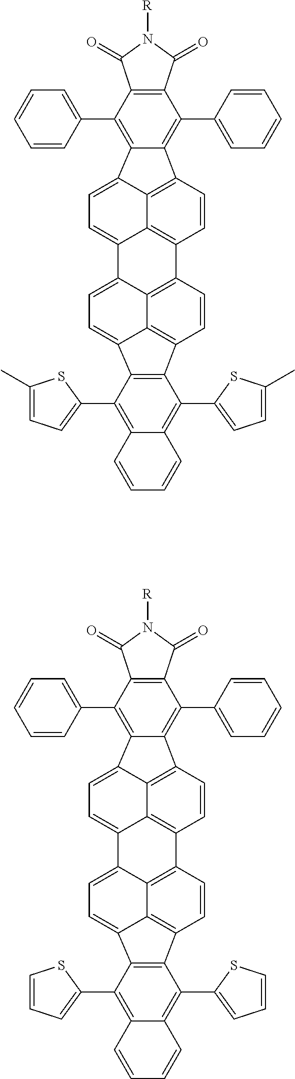

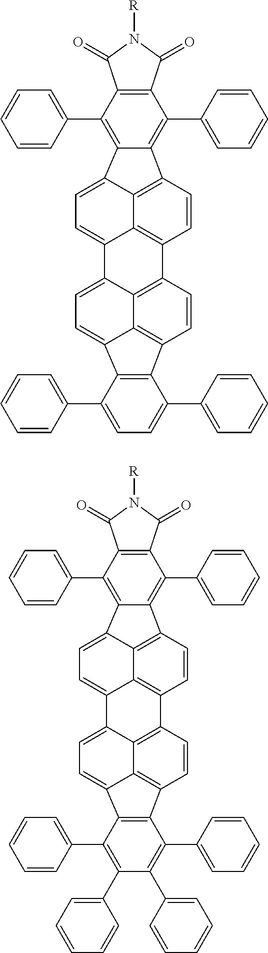

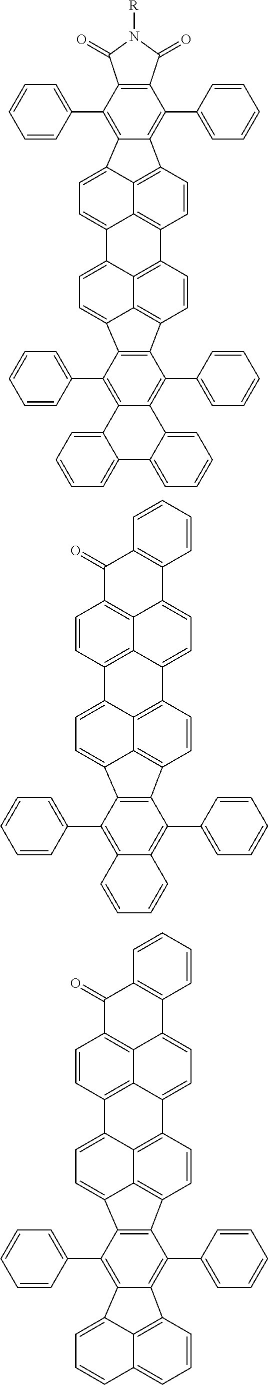

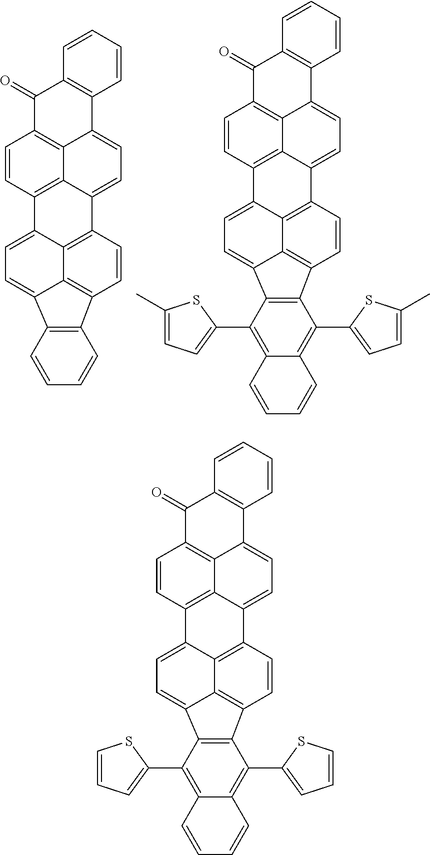

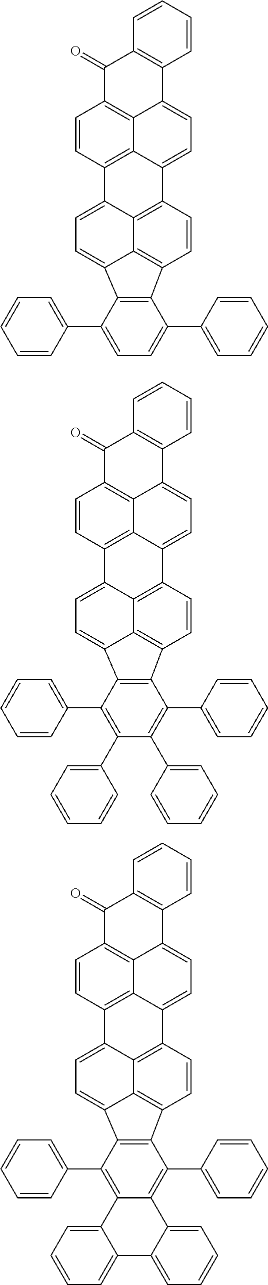

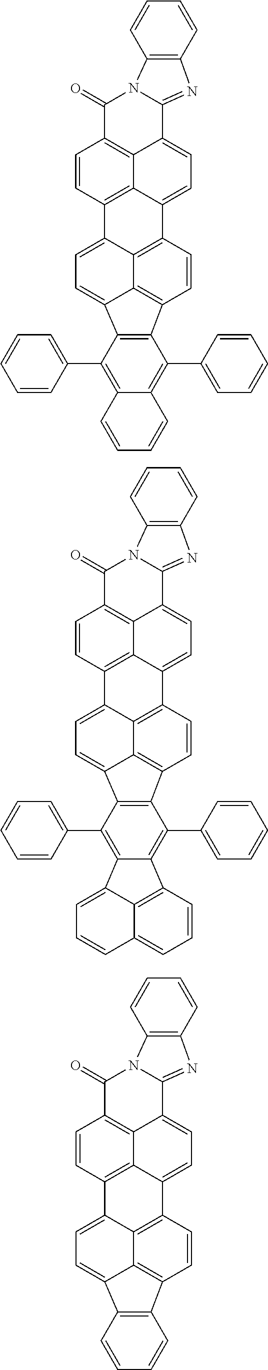

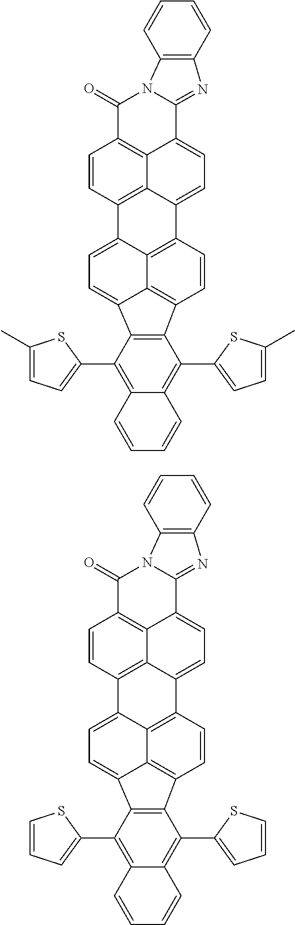

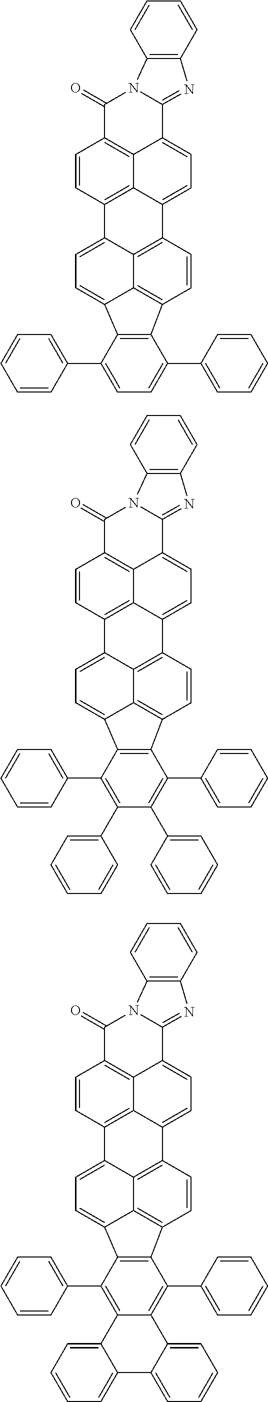

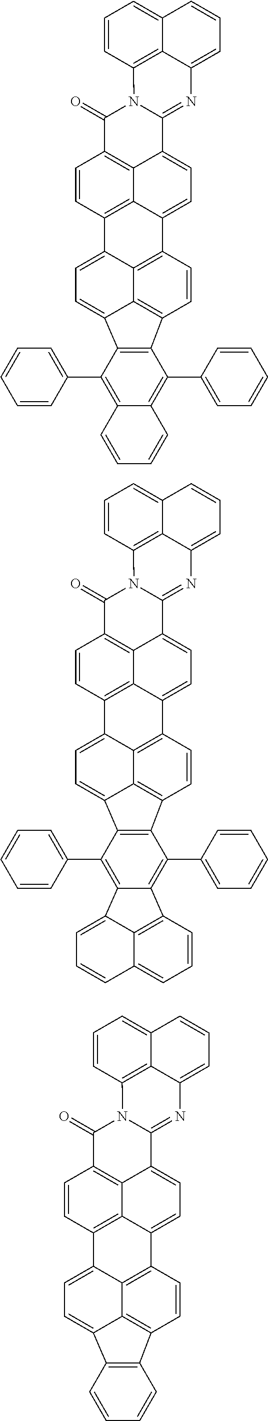

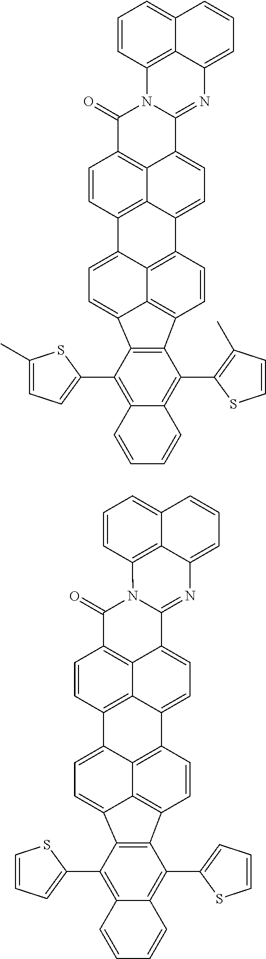

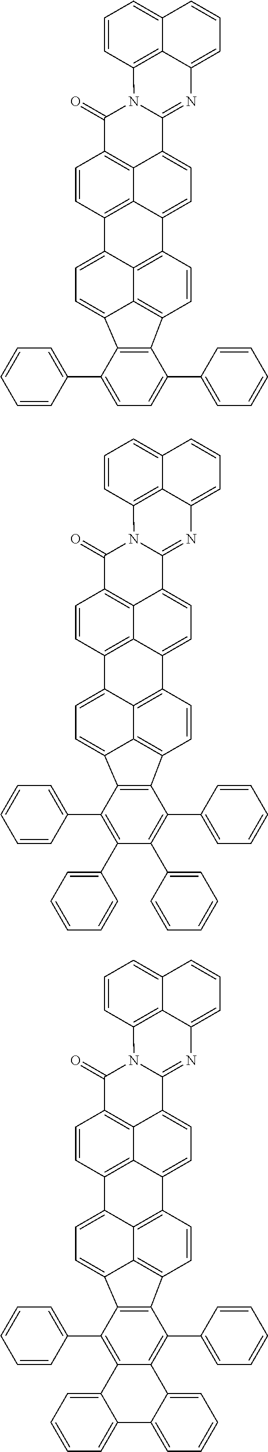

in which
R is hydrogen, C₁-C₁₈-alkyl, C₃-C₇-cycloalkyl, C₃-C₇-heterocyclyl, C₆-C₁₄-aryl-C₁-C₂₀-alkyl, C₆-C₁₄-aryl, C₁-C₂₀-alkyl-C₆-C₁₄-aryl, or a 5- or 6-membered heteroaromatic radical which has 1, 2 or 3 heteroatoms selected from O, S and N as ring members, and in which the heteroaromatic radical may have a fused-on benzene ring or naphthalene ring and may have 1, 2, 3, 4, 5 or 6 R^{b} substituents, and the aryl group in C₆-C₁₄-aryl-C₁-C₂₀-alkyl, C₆-C₁₄-aryl, C₁-C₂₀-alkyl-C₆-C₁₄-aryl may have 1, 2, 3, 4, 5 or 6 R^{b} substituents, and
R^{b} is fluorine, chlorine, cyano, nitro or C₁-C₆-alkoxy.

Especially, R is hydrogen, C₁-C₄-alkyl or phenyl.

The perylenes used in the inventive solar cells can be prepared by customary processes known to those skilled in the art. These include the buildup of the molecular skeleton from a bromaryl compound with an arylboronic acid derivative under the conditions of a Suzuki coupling, i.e. in the presence of a platinum metal catalyst and especially in the presence of a palladium catalyst, under reaction conditions known per se, as known, for example, from Acc. Chem. Res. 15, p. 178-184 (1982), Chem. Rev. 95, p. 2457-2483 (1995), and the literature cited therein, and also from J. Org. Chem. 68, p. 9412 (2003). Suitable catalysts are especially tetrakis(triphenyl-phosphine)palladium(0), bis(triphenylphosphine)palladium(II) chloride, bis(acetonitrile)-palladium(II) chloride, the [1,1′-bis(diphenylphosphino)ferrocene]palladium(II) chloride-dichloromethane complex, bis[1,2-bis(diphenylphosphine)ethane]palladium(0) and [1,4-bis(diphenylphosphine)butane]palladium(II) chloride. The amount of catalyst is typically 0.1 to 10 mol %.

Before use in an organic solar cell, the perylene compound can be subjected to purification. The purification can be effected by customary methods known to those skilled in the art, such as separation on suitable stationary phases, sublimation, extraction, distillation, recrystallization or a combination of at least two of these measures. Each purification may have a one-stage or multistage configuration. Individual purifying operations can be repeated twice or more. Different purifying operations can be combined with one another.

In a specific embodiment, the purification comprises a column chromatography method. To this end, the starting material present in a solvent or solvent mixture can be subjected to a separation or filtration on silica gel. Finally, the solvent is removed, for example by evaporation under reduced pressure. Suitable solvents are aromatics such as benzene, toluene, xylene, mesitylene, chlorobenzene or dichlorobenzene, hydrocarbons and hydrocarbon mixtures, such as pentane, hexane, ligroin and petroleum ether, halogenated hydrocarbons such as chloroform or dichloromethane, and mixtures of the solvents mentioned. For chromatography, it is also possible to use a gradient of at least two different solvents, for example a toluene/petroleum ether gradient.

In a further specific embodiment, the purification comprises a sublimation. This may preferably be a fractional sublimation. For fractional sublimation, it is possible to use a temperature gradient in the sublimation and/or the deposition of the substituted perylene. In addition, the purification can be effected by sublimation with the aid of a carrier gas stream. Suitable carrier gases are inert gases, for example nitrogen, argon or helium. The gas stream laden with the compound can subsequently be passed into a separating chamber. Suitable separating chambers may have a plurality of separation zones which can be operated at different temperatures. Preference is given, for example, to a so-called three-zone sublimation apparatus. A further process and an apparatus for fractional sublimation are described in U.S. Pat. No. 4,036,594.

Organic solar cells generally have a layer structure and generally comprise at least the following layers: anode, photoactive layer and cathode. These layers are generally applied to a substrate suitable for this purpose. The structure of organic solar cells is described, for example, in US 2005/0098726 and US 2005/0224905.

The invention provides an organic solar cell which comprises a substrate with at least one cathode and at least one anode, and at least one perylene compound of the general formula (I) as defined above as a photoactive material. The inventive organic solar cell comprises at least one photoactive region. A photoactive region may comprise two layers, each of which has a homogeneous composition and forms a flat donor-acceptor heterojunction. A photoactive region may also comprise a mixed layer and form a donor-acceptor heterojunction in the form of a donor-acceptor bulk heterojunction. Organic solar cells with photoactive donor-acceptor transitions in the form of a bulk heterojunction are a preferred embodiment of the invention.

Suitable substrates for organic solar cells are, for example, oxidic materials, polymers and combinations thereof. Preferred oxidic materials are selected from glass, ceramic, SiO₂, quartz, etc. Preferred polymers are selected from polyethylene terephthalates, polyolefins (such as polyethylene and polypropylene), polyesters, fluoropolymers, polyamides, polyurethanes, polyalkyl(meth)acrylates, polystyrene, polyvinyl chlorides and mixtures and composites.

Suitable electrodes (cathode, anode) are in principle metals, semiconductors, metal alloys, semiconductor alloys and combinations thereof. Preferred metals are those of groups 2, 8, 9, 10, 11 or 13 of the periodic table, e.g. Pt, Au, Ag, Cu, Al, In, Mg or Ca. Preferred semiconductors are e.g. doped Si, doped Ge, indium tin oxide (ITO), fluorinated tin oxide (FTO), gallium indium tin oxide (GITO), zinc indium tin oxide (ZITO), etc.). Preferred metal alloys are for example alloys based on Pt, Au, Ag, Cu, etc. A specific embodiment is that of Mg/Ag alloys).

The material used for the electrode facing the light (the anode in a normal structure, the cathode in an inverse structure) is preferably a material at least partly transparent to the incident light. This preferably includes electrodes which have glass and/or a transparent polymer as the carrier material. Transparent polymers suitable as carriers are those mentioned above, such as polyethylene terephthalate. The electrical contact connection is generally effected by means of metal layers and/or transparent conductive oxides (TCOs). These preferably include ITO, doped ITO, FTO (fluorine doped tin oxide), AZO (aluminum doped tin oxide), ZnO, TiO₂, Ag, Au, Pt. Particular preference is given to ITO for contact connection. It is also possible to use a conductive polymer for electrical contact connection, for example a poly-3,4-alkylenedioxy-thiophene, e.g. poly-3,4-ethyleneoxythiophene (PEDOT).

The electrode facing the light is configured such that it is sufficiently thin to bring about only minimal light absorption but thick enough to enable good charge transport of the extracted charge carriers. The thickness of the electrode layer (without carrier material) is preferably within a range from 20 to 200 nm.

In a specific embodiment, the material used for the electrode facing away from the light (the cathode in a normal structure, the anode in an inverse structure) is a material which at least partly reflects the incident light. This includes metal films, preferably of Ag, Au, Al, Ca, Mg, In, and mixtures thereof. Preferred mixtures are Mg/Al. The thickness of the electrode layer is preferably within a range from 50 to 300 nm.

The photoactive region comprises or consists of at least one layer which comprises at least one perylene compound of the general formula (I) as defined above. In addition, the photoactive region may have one or more further layer(s). These are, for example, selected from
layers with electron-conducting properties (electron transport layer, ETL),
layers which comprise a hole-conducting material (hole transport layer, HTL), which need not absorb any radiation,
exciton- and hole-blocking layers (e.g. EBLs), which must not absorb, and multiplication layers.

Suitable materials for these layers are described in detail hereinafter. Suitable exciton- and hole-blocking layers are described, for example, in U.S. Pat. No. 6,451,415. Suitable materials for exciton-blocking layers are, for example, bathocuproin (BCP), 4,4′,4″-tris-[3-methylphenyl-N-phenylamino]triphenylamine(m-MTDATA) or polyethylenedioxy-thiophene (PEDOT).

The inventive solar cells comprise at least one photoactive donor-acceptor heterojunction. Optical excitation of an organic material generates excitons. In order that a photocurrent occurs, the electron-hole pair has to be separated, typically at a donor-acceptor interface between two unlike contact materials. At such an interface, the donor material forms a heterojunction with an acceptor material. When the charges are not separated, they can recombine in a process also known as “quenching”, either radiatively by the emission of light of a lower energy than the incident light or nonradiatively by generation of heat. Both processes are undesired. According to the invention, at least one substituted perylene of the general formula (I) can be used as a charge generator (donor). In combination with an appropriate electron acceptor material (ETM, electron transport material), radiative excitation is followed by a rapid electron transfer to the ETM. Suitable ETMs are, for example, C60 and other fullerenes, perylene-3,4,9,10-bis(dicarboxim ides) (PTCDIs), etc. (as described hereinafter). Preferred ETMs are C60 and other fullerenes which are discussed below.

In a first embodiment, the heterojunction has a flat configuration (see: Two layer organic photovoltaic cell, C. W. Tang, Appl. Phys. Lett., 48 (2), 183-185 (1986) or N. Karl, A. Bauer, J. Holzäpfel, J. Marktanner, M. Möbus, F. Stölzle, Mol. Cryst. Liq. Cryst., 252, 243-258 (1994).).

In a second, preferred embodiment, the heterojunction is configured as a bulk (mixed) heterojunction, also referred to as an interpenetrating donor-acceptor network. Organic photovoltaic cells with a bulk heterojunction are described, for example, by C. J. Brabec, N. S. Sariciftci, J. C. Hummelen in Adv. Funct. Mater., 11 (1), 15 (2001) or by J. Xue, B. P. Rand, S. Uchida and S. R. Forrest in J. Appl. Phys. 98, 124903 (2005). Bulk heterojunctions are discussed in detail hereinafter.

The compounds of the formula (I) can be used as a photoactive material in cells with MiM, pin, pn, Mip or Min structure (M=metal, p=p-doped organic or inorganic semiconductor, n=n-doped organic or inorganic semiconductor, i=intrinsically conductive system of organic layers; see, for example, J. Drechsel et al., Org. Electron., 5 (4), 175 (2004) or Maennig et al., Appl. Phys. A 79, 1-14 (2004)).

The compounds of the formula (I) can also be used as a photoactive material in tandem cells. Suitable tandem cells are described, for example, by P. Peumans, A. Yakimov, S. R. Forrest in J. Appl. Phys, 93 (7), 3693-3723 (2003) (see also U.S. Pat. No. 4,461,922, U.S. Pat. No. 6,198,091 and U.S. Pat. No. 6,198,092) and are described in detail hereinafter. The use of perylene compounds of the general formula (I) in tandem cells is a preferred embodiment of the invention.

The compounds of the formula (I) can also be used as a photoactive material in tandem cells which are constructed from two or more than two stacked MiM, pin, Mip or Min structures (see DE 103 13 232.5 and J. Drechsel et al., Thin Solid Films, 451452, 515-517 (2004)).

The layer thickness M, n, i and p layers is typically within a range from 10 to 1000 nm, more preferably from 10 to 400 nm. The layers which form the solar cell can be produced by customary processes known to those skilled in the art. These include vapor deposition under reduced pressure or in an inert gas atmosphere, laser ablation or solution or dispersion processing methods such as spincoating, knifecoating, casting methods, spray application, dipcoating or printing (e.g. inkjet, flexographic, offset, gravure; intaglio, nanoimprinting). In a specific embodiment, the entire solar cell is produced by a gas phase deposition process.

In order to improve the efficiency of organic solar cells, it is possible to shorten the mean distance through which the exciton has to diffuse in order to arrive at the next donor-acceptor interface. To this end, it is possible to use mixed layers of donor material and acceptor material which form an interpenetrating network in which internal donor-acceptor heterojunctions are possible. This is known as a bulk heterojunction, a specific form of the mixed layer in which the excitons generated need only travel a very short distance before they arrive at a domain boundary, where they are separated.

In a preferred embodiment, the photoactive donor-acceptor transitions in the form of a bulk heterojunction are produced by a gas phase deposition process (physical vapor deposition, PVD). Suitable processes are described, for example, in US 2005/0227406, to which reference is made here. To this end, a perylene compound of the general formula (I) and a complementary semiconductor material can be subjected to a gas phase deposition in the manner of a cosublimation. PVD processes are performed under high-vacuum conditions and comprise the following steps: evaporation, transport, deposition. The deposition is effected preferably at a pressure within a range from about 10⁻² mbar to 10⁻⁷ mbar, for example from 10⁻⁵ to 10⁻⁷ mbar. The deposition rate is preferably within a range from 0.01 to 10 nm/s. The deposition can be effected in an inert gas atmosphere, for example under nitrogen, helium or argon. The temperature of the substrate during the deposition is preferably within a range from −100 to 300° C., more preferably from −50 to 250° C.

The other layers of the organic solar cell can be produced by known processes. These include vapor deposition under reduced pressure or in an inert gas atmosphere, laser ablation or solution or dispersion processing methods, such as spin-coating, knife-coating, casting processes, spray application, dip-coating or printing (e.g. inkjet, flexographic, offset, gravure; intaglio printing, nanoimprinting). In a specific embodiment, the entire solar cell is produced by a gas phase deposition process.

The photoactive layer (homogeneous layer or mixed layer) can be subjected to a thermal treatment directly after production thereof or after production of further layers which form the solar cell. Such a heat treatment can in many cases further improve the morphology of the photoactive layer. The temperature is preferably within a range from about 60° C. to 300° C. The treatment time is preferably within a range from 1 minute to 3 hours. In addition or alternatively to a thermal treatment, the photoactive layer (mixed layer) can be subjected to a treatment with a solvent-containing gas directly after production thereof or after production of further layers which form the solar cell. In a suitable embodiment, saturated solvent vapors in air are used at ambient temperature. Suitable solvents are toluene, xylene, chloroform, N-methylpyrrolidone, dimethylformamide, ethyl acetate, chlorobenzene, dichloromethane and mixtures thereof. The treatment time is preferably within a range from 1 minute to 3 hours.

In a preferred embodiment, the inventive solar cells are present as an individual cell with a flat heterojunction and normal structure. FIG. 1 shows an inventive solar cell with normal structure. In a specific embodiment, the cell has the following structure:
an at least partly transparent conductive layer (top electrode, anode) (11)
a hole-conducting layer (hole transport layer, HTL) (12)
a layer which comprises a donor material (13)
a layer which comprises an acceptor material (14)
an exciton-blocking and/or electron-conducting layer (15)
a second conductive layer (back electrode, cathode) (16)

The donor material preferably comprises at least one compound of the formula (I) or consists of a compound of the formula (I). The acceptor material preferably comprises at least one fullerene or fullerene derivative, or consists of a fullerene or fullerene derivative. The acceptor material preferably comprises C60 or PCBM ([6,6]-phenyl-C61-butyric acid methyl ester).

The essentially transparent conductive layer (11) (anode) comprises a carrier, such as glass or a polymer (e.g. polyethylene terephthalate) and a conductive material, as described above. Examples include ITO, doped ITO, FTO, ZnO, AZO, etc. The anode material can be subjected to a surface treatment, for example with UV light, ozone, oxygen plasma, Br₂, etc. The layer (11) should be sufficiently thin to enable maximum light absorption, but also sufficiently thick to ensure good charge transport. The layer thickness of the transparent conductive layer (11) is preferably within a range from 20 to 200 nm.

The solar cell with normal structure according to FIG. 1 optionally has a hole-conducting layer (HTL). This layer comprises at least one hole-conducting material (hole transport material, HTM). Layer (12) may be an individual layer of essentially homogeneous composition or may comprise two or more than two sublayers.

Hole-conducting materials (HTM) suitable for forming layers with hole-conducting properties (HTL) preferably comprise at least one material with high ionization energy. The ionization energy is preferably at least 5.0 eV, more preferably at least 5.5 eV. The materials may be organic or inorganic materials. Organic materials suitable for use in a layer with hole-conducting properties are preferably selected from poly(3,4-ethylene-dioxythiophene)poly(styrenesulfonate) (PEDOT-PSS), Ir-DPBIC (tris-N,N′-diphenyl-benzimidazol-2-ylideneiridium(III)), N,N′-diphenyl-N,N′-bis(3-methylphenyl)-1,1′-diphenyl-4,4′-diamine(α-NPD), 2,2′,7,7′-tetrakis(N,N-di-p-methoxyphenylamine)-9,9′-spirobifluorene (spiro-MeOTAD), etc. and mixtures thereof. The organic materials may, if desired, be doped with a p-dopant which has a LUMO within the same range as or lower than the HOMO of the hole-conducting material. Suitable dopants are, for example, 2,3,5,6-tetrafluoro-7,7,8,8-tetracyanoquinodimethane (F₄TCNQ), WO₃, MoO₃, etc. Inorganic materials suitable for use in a layer with hole-conducting properties are preferably selected from WO₃, MoO₃, etc.

If present, the thickness of the layers with hole-conducting properties is preferably within a range from 5 to 200 nm, more preferably 10 to 100 nm.

Layer (13) comprises at least one compound of the general formula (I). The thickness of the layer should be sufficient to absorb a maximum amount of light, but thin enough to enable effective dissipation of the charge. The thickness of the layer (13) is preferably within a range from 5 nm to 1 μm, more preferably from 5 to 80 nm.

Layer (14) comprises at least one acceptor material. The acceptor material preferably comprises at least one fullerene or fullerene derivative. Alternatively or additionally suitable acceptor materials are specified hereinafter. The thickness of the layer should be sufficient to absorb a maximum amount of light, but thin enough to enable effective dissipation of the charge. The thickness of the layer (14) is preferably within a range from 5 nm to 1 μm, more preferably from 5 to 80 nm.

The solar cell with normal structure according to FIG. 1 optionally comprises an exciton-blocking and/or electron-conducting layer (15) (EBL/ETL). Suitable materials for exciton-blocking layers generally have a greater band gap than the materials of layer (13) and/or (14). They are firstly capable of reflecting excitons and secondly enable good electron transport through the layer. The materials for the layer (15) may comprise organic or inorganic materials. Suitable organic materials are preferably selected from 2,9-dimethyl-4,7-diphenyl-1,10-phenanthroline (BCP), 4,7-diphenyl-1,10-phenanthroline (Bphen), 1,3-bis[2-(2,2′-bipyridin-6-yl)-1,3,4-oxadiazo-5-yl]benzene (BPY-OXD), etc. The organic materials may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. Inorganic materials suitable for use in a layer with electron-conducting properties are preferably selected from ZnO, etc. If present, the thickness of the layer is preferably within a range from 5 to 500 nm, more preferably 10 to 100 nm.

Layer 16 is the cathode and preferably comprises at least one compound with low work function, more preferably a metal such as Ag, Al, Mg, Ca, etc. The thickness of the layer (16) is preferably within a range from about 10 nm to 10 μm, e.g. 10 nm to 60 nm.

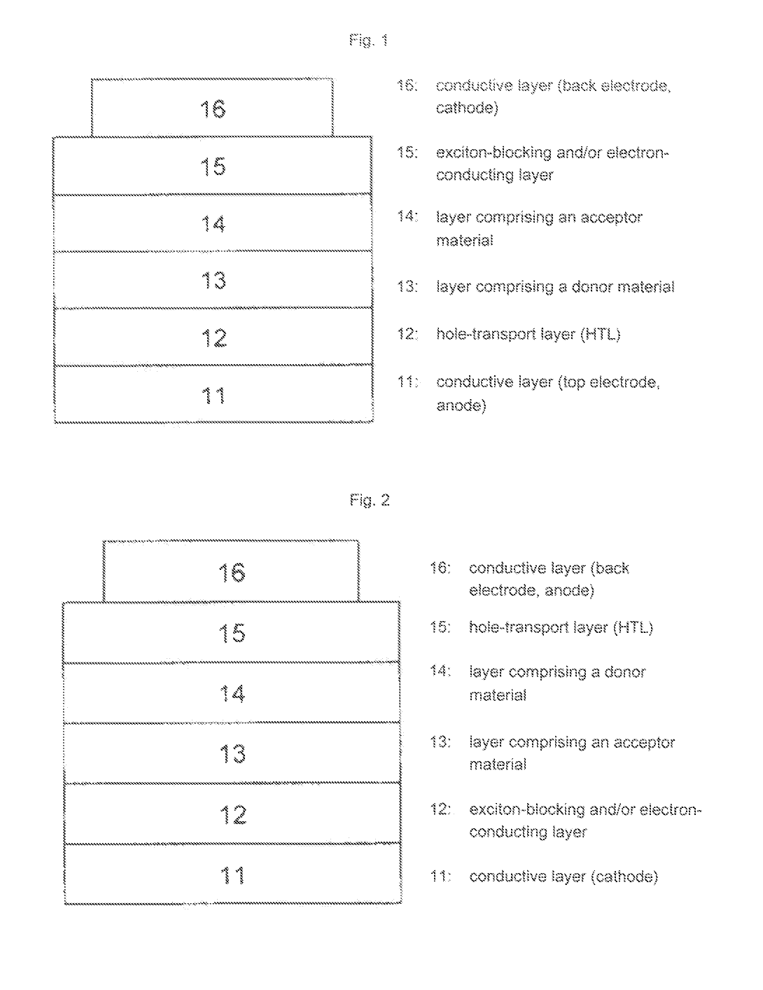

In a preferred embodiment, the inventive solar cells are present as an individual cell with a flat heterojunction and inverse structure. FIG. 2 shows a solar cell with inverse structure. In a specific embodiment, the cell has the following structure:
an at least partly transparent conductive layer (cathode) (11)
an exciton-blocking and/or electron-conducting layer (12)
a layer which comprises an acceptor material (13)
a layer which comprises a donor material (14)
a hole-conducting layer (hole transport layer, HTL) (15)
a second conductive layer (back electrode, anode) (16)

With regard to suitable and preferred materials for the layers (11) to (16), reference is made to the above remarks regarding the corresponding layers in solar cells with normal structure.

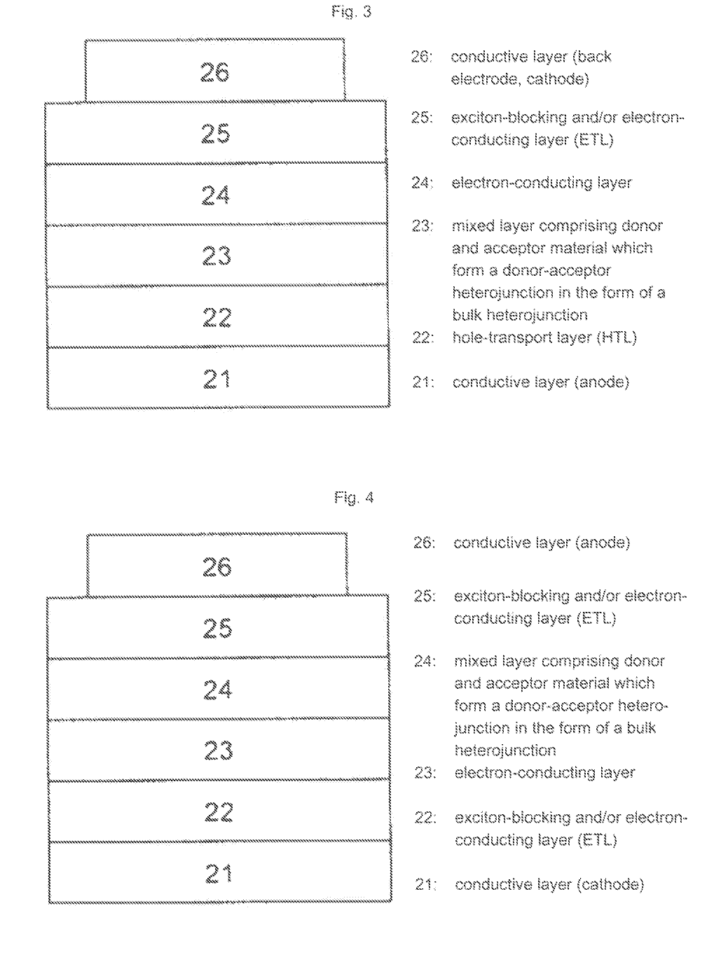

In a further preferred embodiment, the inventive solar cells are present as an individual cell with normal structure and have a bulk heterojunction. FIG. 3 shows a solar cell with a bulk heterojunction. In a specific embodiment, the cell has the following structure:
an at least partly transparent conductive layer (anode) (21)
a hole-conducting layer (hole transport layer, HTL) (22)
a mixed layer which comprises a donor material and an acceptor material, which
form a donor-acceptor heterojunction in the form of a bulk heterojunction (23)
an electron-conducting layer (24)
an exciton-blocking and/or electron-conducting layer (25)
a second conductive layer (back electrode, cathode) (26)

The layer (23) comprises at least one perylene compound of the general formula (I) as a photoactive material, especially as a donor material. The layer (23) additionally comprises preferably at least one fullerene or fullerene derivative as an acceptor material. The layer (23) comprises especially C60 or PCBM ([6,6]-phenyl-C61-butyric acid methyl ester) as an acceptor material.

With regard to layer (21), reference is made completely to the above remarks regarding layer (11).

With regard to layer (22), reference is made completely to the above remarks regarding layer (12).

Layer (23) is a mixed layer which comprises at least one compound of the general formula (I) as a donor material. In addition, layer (23) comprises at least one acceptor material. As described above, the layer (23) can be produced by coevaporation or by solution processing using customary solvents. The mixed layer comprises preferably 10 to 90% by weight, more preferably 20 to 80% by weight, of at least one compound of the general formula (I), based on the total weight of the mixed layer. The mixed layer comprises preferably 10 to 90% by weight, more preferably 20 to 80% by weight, of at least one acceptor material, based on the total weight of the mixed layer. The thickness of the layer (23) should be sufficient to absorb a maximum amount of light, but thin enough to enable effective dissipation of the charge. The thickness of the layer (23) is preferably within a range from 5 nm to 1 μm, more preferably from 5 to 200 nm, especially 5 to 80 nm.

The solar cell with a bulk heterojunction according to FIG. 3 comprises an electron-conducting layer (24) (ETL). This layer comprises at least one electron transport material (ETM). Layer (24) may be a single layer of essentially homogeneous composition or may comprise two or more than two sublayers. Suitable materials for electron-conducting layers generally have a low work function or ionization energy. The ionization energy is preferably not more than 3.5 eV. Suitable organic materials are preferably selected from the aforementioned fullerenes and fullerene derivatives, 2,9-dimethyl-4,7-diphenyl-1,10-phenanthroline (BCP), 4,7-diphenyl-1,10-phenanthroline (Bphen), 1,3-bis[2-(2,2-bipyridin-6-yl)-1,3,4-oxadiazo-5-yl]benzene (BPY-OXD), etc. The organic materials used in layer (24) may, if desired, be doped with an n-dopant which has a HOMO within the same range as or lower than the LUMO of the electron-conducting material. Suitable dopants are, for example, Cs₂CO₃, Pyronin B (PyB), Rhodamine B, cobaltocenes, etc. The thickness of the layer (23) is, if present, preferably within a range from 1 nm to 1 μm, particularly 5 to 60 nm.

With regard to layer (25), reference is made completely to the above remarks regarding layer (15).

With regard to layer (26), reference is made completely to the above remarks regarding layer (16).

The solar cell with a donor-acceptor heterojunction in the form of a bulk heterojunction can be produced by a gas phase deposition process as described above. With regard to deposition rates, substrate temperature during the deposition and thermal aftertreatment, reference is made to the above remarks.

In a further preferred embodiment, the inventive solar cells are present as an individual cell with inverse structure and have a bulk heterojunction. FIG. 4 shows a solar cell with a bulk heterojunction and inverse structure.

In a further, particularly preferred embodiment, the inventive solar cell is a tandem cell.

A tandem cell consists of two or more than two (e.g. 3, 4, 5, etc.) subcells. A single subcell, some of the subcells or all subcells may have photoactive donor-acceptor heterojunctions. Each donor-acceptor-heterojunction may be in the form of a flat heterojunction or in the form of a bulk hetero-junction. Preferably, at least one of the donor-acceptor heterojunctions is in the form of a bulk heterojunction. According to the invention, the photoactive layer of at least one subcell comprises a perylene compound periflanthene of the general formula (I). Preferably, the photoactive layer of at least one subcell comprises a perylene compound of the general formula (I) and at least one fullerene or fullerene derivative. More preferably, the semiconductor mixture used in the photoactive layer of at least one subcell consists of a perylene compound of the general formula (I) and C₆₀ or [6,6]-phenyl-C61-butyric acid methyl ester.

The subcells which form the tandem cell may be connected in parallel or in series. The subcells which form the tandem cell are preferably connected in series. There is preferably an additional recombination layer in each case between the individual subcells. The individual subcells have the same polarity, i.e. generally either only cells with normal structure or only cells with inverse structure are combined with one another.

FIG. 5 shows the basic structure of an inventive tandem cell. Layer 31 is a transparent conductive layer. Suitable materials are those specified above for the individual cells.

Layers 32 and 34 constitute subcells. “Subcell” refers here to a cell as defined above without cathode and anode. The subcells may, for example, either all have a perylene compound of the general formula (I) used in accordance with the invention in the photoactive layer (preferably in combination with a fullerene or fullerene derivative, especially C60) or have other combinations of semiconductor materials, for example C60 with zinc phthalocyanine, C60 with oligothiophene (such as DCV5T). In addition, individual subcells may also be configured as dye-sensitized solar cells or polymer cells. In all cases, preference is given to a combination of materials which exploit different regions of the spectrum of the incident light, for example of natural sunlight. For instance, the combination of perylene compound of the general formula (I) and fullerene or fullerene derivative used in accordance with the invention absorbs in the short-wave range of sunlight, e.g. in the range from 400 to 600 nm. According to a preferred embodiment, the tandem cell comprises at least one subcell with an absorption maximum in the relatively long-wave spectral region, e.g. in the range from 600 nm to 800 nm. Zinc phthalocyanine-C60 cells absorb primarily in the range from 600 nm to 800 nm. Thus, a tandem cell composed of a combination of these subcells should absorb radiation in the range from about 400 nm to 800 nm.

According to a further preferred embodiment, the tandem cell comprises at least one subcell with an absorption maximum in the range from 400 to 600 nm and at least one subcell with an absorption maximum in the range from 600 to 850 nm. According to a further preferred embodiment, the tandem cell comprises at least one subcell with an absorption maximum in the range from 400 to 550 nm and at least one subcell with an absorption maximum in the range from 550 to 900 nm. According to a further preferred embodiment, the tandem cell comprises at least one subcell with an absorption maximum in the range from 400 to 650 nm and at least one subcell with an absorption maximum in the range from 650 to 900 nm. Suitable combination of subcells should thus allow the spectral range utilized to be extended. For optimal performance properties, optical interference should be considered. For instance, subcells which absorb at relatively short wavelengths should be arranged closer to the metal top contact than subcells with longer-wave absorption. According to a preferred embodiment, the tandem cell additionally comprises at least one subcell with an absorption maximum in the relatively long-wave spectral region, preferably in the range from 600 to 900 nm.

With regard to layer (31), reference is made completely to the above remarks regarding layers (11) and (21).

With regard to layers (32) and (34), reference is made completely to the above remarks regarding layers (12) to (15) for flat heterojunctions and (22) to (25) for bulk heterojunctions.

Layer 33 is a recombination layer. Recombination layers enable the charge carriers from one subcell to recombine with those of an adjacent subcell. Small metal clusters are suitable, such as Ag, Au or combinations of highly n- and p-doped layers. In the case of metal clusters, the layer thickness is preferably within a range from 0.5 to 5 nm. In the case of highly n- and p-doped layers, the layer thickness is preferably within a range from 5 to 40 nm. The recombination layer generally connects the electron-conducting layer of a subcell to the hole-conducting layer of an adjacent subcell. In this way, further cells can be combined to form the tandem cell.

Layer 36 is the top electrode. The material depends on the polarity of the subcells. For subcells with normal structure, preference is given to using metals with a low work function, such as Ag, Al, Mg, Ca, etc. For subcells with inverse structure, preference is given to using metals with a high work function, such as Au or Pt, or PEDOT-PSS.

In the case of subcells connected in series the overall voltage corresponds to the sum of the individual voltages of all subcells. The overall current, in contrast, is limited by the lowest current of one subcell. For this reason, the thickness of each subcell should be optimized such that all subcells have essentially the same current.

Examples of different kinds of donor-acceptor heterojunctions are a donor-acceptor double layer with a flat heterojunction, or the heterojunction is configured as a hybrid planar-mixed heterojunction or gradient bulk heterojunction or annealed bulk heterojunction.

The production of a hybrid planar-mixed heterojunction is described in Adv. Mater. 17, 66-70 (2005). In this structure, mixed heterojunction layers which were formed by simultaneous evaporation of acceptor and donor material are present between homogeneous donor and acceptor material.

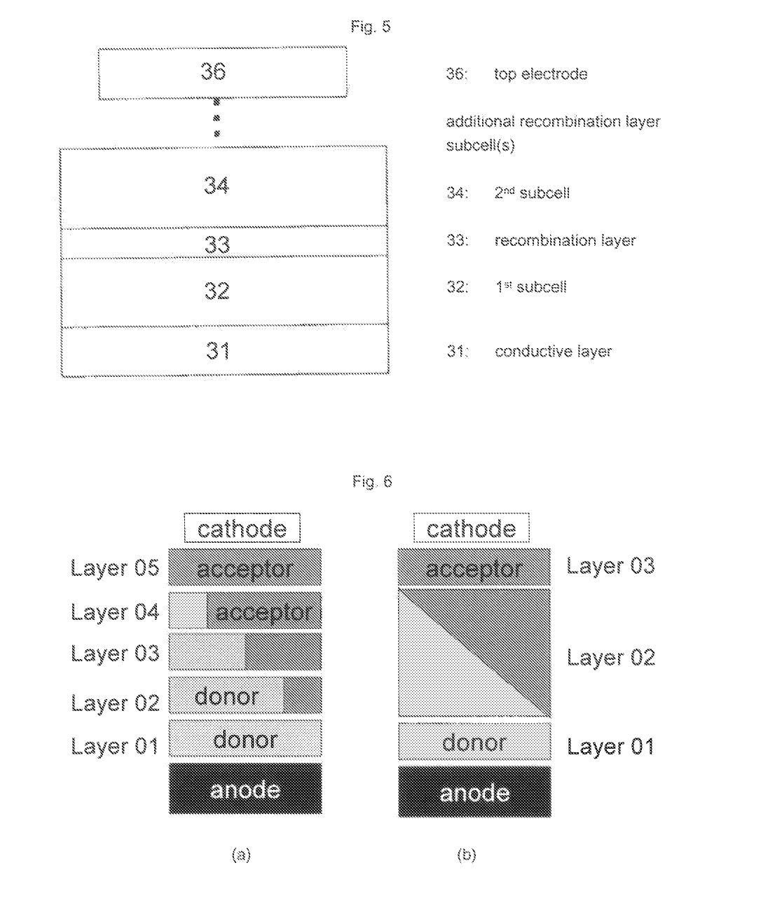

In a specific embodiment of the present invention, the donor-acceptor-heterojunction is in the form of a gradient bulk heterojunction. In the mixed layers composed of donor and acceptor materials, the donor-acceptor ratio changes gradually. The form of the gradient may be stepwise (FIG. 6(a)) or linear (FIG. 6(b)). In FIG. 6 (a), the layer 01 consists of 100% donor material, layer 02 has a donor/acceptor ratio >1, layer 03 has a donor/acceptor ratio=1, layer 04 has a donor/acceptor ratio <1, and layer 05 consists of 100% acceptor material. In FIG. 6 (b), layer 01 consists of 100% donor material, layer 02 has a decreasing ratio of donor/acceptor, i.e. the proportion of donor material decreases in a linear manner in the direction of layer 03, and layer 03 consists of 100% acceptor material. The different donor-acceptor ratios can be controlled by means of the deposition rate of each and every material. Such structures can promote the percolation path for charges.

In a further specific embodiment of the present invention, the donor-acceptor-heterojunction is configured as an annealed bulk heterojunction; see, for example, Nature 425, 158-162, 2003. The process for producing such a solar cell comprises an annealing step before or after the metal deposition. As a result of the annealing, donor and acceptor materials can separate, which leads to more extended percolation paths.

In a further specific embodiment of the present invention, the organic solar cells are produced by organic vapor phase deposition, either with a flat or a controlled heterojunction architecture. Solar cells of this type are described in Materials, 4, 2005, 37.

In a specific embodiment, at least one substituted perylene of the general formula (I) is used as the sole electron donor material.

The inventive organic solar cells comprise at least one photoactive region which comprises at least one perylene compound as a donor which is in contact with at least one acceptor. Preferred acceptors are fullerenes and fullerene derivatives, preferably selected from C₆₀, C₇₀, C₈₄, phenyl-C₆₁-butyric acid methyl ester ([60]PCBM), phenyl-C₇₁-butyric acid methyl ester ([71]PCBM), phenyl-C₈₄-butyric acid methyl ester ([84]PCBM), phenyl-C₆₁-butyric acid butyl ester ([60]PCBB), phenyl-C₆₁-butyric acid octyl ester ([60]PCBO), thienyl-C₆₁-butyric acid methyl ester ([60]ThCBM) and mixtures thereof. Particular preference is given to C₆₀, [60]PCBM and mixtures thereof. Likewise, particular preference is given to C₇₀.

In addition to perylene compounds of the formula (I) and fullerenes, the semiconductor materials listed hereinafter are suitable in principle for use in the inventive solar cells. They serve especially as donors or acceptors for subcells of a tandem cell, which are combined with a perylene/fullerene subcell used in accordance with the invention.

Suitable further semiconductors are phthalocyanines. These include phthalocyanines which are unhalogenated or which bear 1 to 16 halogen atoms. The phthalocyanines may be metal-free or comprise a divalent metal or a metal atom-containing group. Preference is given to phthalocyanines based on zinc, copper, iron, titanyloxy, vanadyloxy, etc. Particular preference is given to copper phthalocyanines, zinc phthalocyanines and metal-free phthalocyanines. In a specific embodiment, a halogenated phthalocyanine is used. These include:

2,6,10,14-tetrafluorophthalocyanines, e.g. copper 2,6,10,14-tetrafluorophthalocyanine and zinc 2,6,10,14-tetrafluorophthalocyanine;
1,5,9,13-tetrafluorophthalocyanines, e.g. copper 1,5,9,13-tetrafluorophthalocyanine and zinc 1,5,9,13-tetrafluorophthalocyanine;
2,3,6,7,10,11,14,15-octafluorophthalocyanine, e.g. copper 2,3,6,7,10,11,14,15-octafluorophthalocyanine and zinc 2,3,6,7,10,11,14,15-octafluorophthalocyanine;

Phthalocyanines which are suitable as acceptors, are, for example, hexadecachlorophthalocyanines and hexadecafluorophthalocyanines, such as copper hexadecachlorophthalocyanine, zinc hexadecachlorophthalocyanine, metal-free hexadecachlorophthalocyanine, copper hexadecafluorophthalocyanine, zinc hexadecafluorophthalocyanine or metal-free hexadecafluorophthalocyanine.

Suitable further semiconductors which are used predominantly as acceptors are rylenes other than the perylene compounds of the formula (I) used in accordance with the invention. In the context of the invention, rylenes are generally understood to mean compounds with a molecular structure of peri-linked naphthalene units. According to the number of naphthalene units, the compounds may, for example, be perylenes (n=2), terrylenes (n=3), quaterrylenes (n=4) or higher rylenes. Accordingly, they may be perylenes, terrylenes or quaterrylenes of the following formulae.

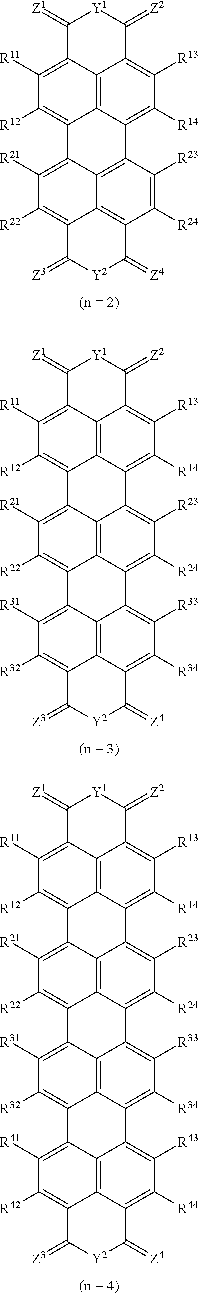

in which
the Rⁿ¹, Rⁿ², Rⁿ³ and Rⁿ⁴ radicals for n=1 to 4 may each independently be hydrogen, halogen or groups other than halogen,
Y¹ is O or NR^{a} where R^{a} is hydrogen or an organyl radical,
Y² is O or NR^{b} where R^{b} is hydrogen or an organyl radical,
Z¹, Z², Z³ and Z⁴ are each O,
where, in the case that Y¹ is NR^{a}, one of the Z¹ and Z² radicals may also be NR^{c}, where the R^{a} and R^{c} radicals together are a bridging group having 2 to 5 atoms between the flanking bonds, and
where, in the case that Y² is NR^{b}, one of the Z³ and Z⁴ radicals may also be NR^{d}, where the R^{b} and R^{d} radicals together are a bridging group having 2 to 5 atoms between the flanking bonds.

Suitable rylenes are, for example, described in WO 2007/074137, WO 2007/093643 and WO 2007/116001, to which reference is made here.

Also suitable are the following donor semiconductor materials, which can be used, for example, in a tandem cell as described hereinafter, in a further subcell in place of the perylene compounds (I).

Semiconductors suitable as donors are porphyrins, for example 5,10,15,20-tetra-(3-pyridyl)porphyrin (TpyP), or else tetrabenzoporphyrins, for example metal-free tetrabenzoporphyrin, copper tetrabenzoporphyrin or zinc tetrabenzoporphyrin. Especially preferred are tetrabenzoporphyrins. These can be processed from solution as soluble precursors and are converted to the photoactive component by thermolysis on the substrate.

Further semiconductors suitable as donors are acenes. These are preferably selected from anthracene, tetracene, pentacene, each of which may be unsubstituted or substituted. Substituted acenes preferably comprise at least one substituent which is selected from electron-donating substituents, electron-withdrawing substituents and combinations thereof. Suitable electron-donating substituents are, for example, alkyl, alkoxy, ester, carboxylate or thioalkoxy. Suitable electron-withdrawing substituents are, for example, halogen, nitro or cyano. Preferred acenes are selected from 2,9-dialkyl-pentacenes and 2,10-dialkylpentacenes, 2,10-dialkoxypentacenes, 1,4,8,11-tetra-alkoxypentacenes and rubrene (5,6,11,12-tetraphenylnaphthacene). Suitable substituted pentacenes are described in US 2003/0100779 and U.S. Pat. No. 6,864,396, to which reference is made here. A particularly preferred acene is rubrene.

Further semiconductors suitable as donors are liquid-crystalline materials (LC materials). These are preferably selected from coronenes and triphenylenes. Preferred liquid-crystalline materials are hexabenzocoronene (HBC-PhC₁₂), coronenediimides, 2,3,6,7,10,11-hexahexylthiotriphenylene (HTT₆), 2,3,6,7,10,11-hexakis-(4-n-nonyl-phenyl)triphenylene (PTP₉) or 2,3,6,7,10,11-hexakis(undecyloxy)triphenylene (HAT₁₁). Particular preference is given to liquid-crystalline materials which are discotic.

Further semiconductors suitable as donors are thiophene compounds. These are preferably selected from thiophenes, oligothiophenes and substituted derivatives thereof. Suitable oligothiophenes are quaterthiophenes, quinquethiophenes, sexithiophenes, α,ω-di(C₁-C₈)-alkyloligothiophenes, such as α,ω-dihexylquarter-thiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers.

Further thiophene compounds suitable as semiconductors are preferably selected from compounds of the type

α,α′-bis(2,2-dicyanovinyl)quinquethiophene (DCV5T),
(3-(4-octylphenyl)-2,2′-bithiophene) (PTOPT),
poly-3-(4′-(1,4,7-trioxaoctyl)-phenyl)thiophene (PEOPT),
(poly(3-(2′-methoxy-5′-octylphenyl)thiophene)) (POMeOPT),
poly(3-octylthiophene) (P30T),
poly[2,6-(4,4-bis(2-ethylhexyl)-4H-cyclopenta[2,1b; 3,4b′]dithiophene)-4,7-(2,1,3-benzothiadiazole) (PCPDTBT), and
poly(pyridopyrazinevinylene)-polythiophene blends, such as EHH-PpyPz, PTPTB copolymers, BBL, F₈BT, PFMO (see Brabec C., Adv. Mater., 2996, 18, 2884).

Further semiconductors suitable as donors are paraphenylenevinylene and oligomers or polymers comprising paraphenylenevinylene units. The latter are preferably selected from polyparaphenylenevinylene, MEH-PPV (poly(2-methoxy-5-(2′-ethylhexyloxy)-1,4-phenylenevinylene, MDMO-PPV (poly(2-methoxy-5-(3′,7′-dimethyloctyloxy)-1,4-phenylenevinylene)), PPV, CN-PPV (with various alkoxy derivatives) and phenyleneethynylene/phenylenevinylene hybrid polymers (PPE-PPV).

Further semiconductors suitable as donors are polyfluorenes and alternating polyfluorene copolymers. These are preferably selected from 4,7-dithien-2′-yl-2,1,3-benzothiadiazole, poly(9,9′-dioctylfluorene-co-benzothiadiazole) (F₈BT), and poly(9,9′-dioctylfluorene-co-bis-N,N′-(4-butylphenyl)-bis-N,N′-phenyl-1,4-phenylene-diamine (PFB).

Further semiconductors suitable as donors are polycarbazoles, i.e. oligomers and polymers comprising carbazole.

Further semiconductors suitable as donors are polyanilines, i.e. oligomers and polymers comprising aniline.

Further semiconductors suitable as donors are triarylamines, polytriarylamines, polycyclopentadienes, polypyrroles, polyfurans, polysiloles, polyphospholes, TPD, CBP, spiro-MeOTAD.

In a preferred embodiment, the inventive solar cell has the following layers:

### ITO

perylene compound of the formula (I)

### C60

BPhen (=4,7-diphenyl-1,10-phenanthroline)

### Ag

The inventive solar cell is more preferably a tandem cell. In that case, one subcell preferably has a photoactive region which comprises at least one perylene compound of the formula (I) and C60. According to a further preferred embodiment, one subcell preferably has a photoactive region which comprises at least one perylene compound of the formula (I) and C70. In particular, the subcell has a photoactive region which comprises at least one perylene compound of the formula (I) and C60.

All aforementioned semiconductors may be doped. The conductivity of semiconductors can be increased by chemical doping techniques using dopants. An organic semiconductor material can be doped with an n-dopant which has a HOMO energy level which is close to or higher than the LUMO energy level of the electron-conducting material. An organic semiconductor material may also be doped with a p-dopant which has a LUMO energy level which is close to or higher than the HOMO energy level of the hole-conductive material. In other words, in the case of n-doping, an electron is released from the dopant which acts as a donor, whereas, in the case of p-doping, the dopant acts as an acceptor which absorbs an electron.

Suitable dopants for the perylene compounds used in accordance with the invention and for p-semiconductors in general are, for example, selected from WO₃, MoO₃, 2,3,5,6-tetrafluoro-7,7,8,8-tetracyanoquinodimethane (F₄-TCNQ), 3,6-difluoro-2,5,7,7,8,8-hexacyanoquinodimethane, dichlorodicyanoquinone (DDQ) or tetra-cyanoquinodimethane (TCNQ). A preferred dopant is 3,6-difluoro-2,5,7,7,8,8-hexa-cyanoquinodimethane.

Suitable dopants for the p-semiconductors used in accordance with the invention are, for example, selected from Cs₂CO₃, LiF, pyronin B (PyB), rhodamine derivatives, cobaltocenes, etc. Preferred dopants are pyronin B and rhodamine derivatives, especially rhodamine B.

Typically, the dopants are used in an amount of up to 10 mol %, preferably up to 5 mol %, based on the amount of the semiconductor to be doped.

The invention is illustrated in detail with reference to the nonlimiting examples which follow.

### EXAMPLES

### I. Preparation of Precursors

### Example I.a

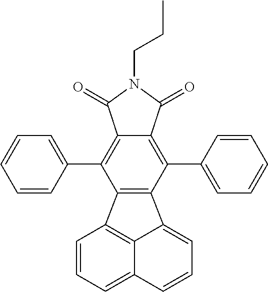

7,9-Diphenyl-8H-cyclopenta[1]acenaphthylene-8-one was prepared according to Müllen et al. Chem. Eur. J. 2001, 7, 10 2197-2205. A mixture of 10.0 g (28 mmol) of 7,9-diphenyl-8H-cyclopenta[1]acenaphthylene-8-one, 9.8 g (52 mmol) of n-propyl-maleimide in 140 ml of chlorobenzene was heated at reflux for three hours. After cooling to room temperature, a solution of 5.8 g (37 mmol) of potassium permanganate and 9.7 mg (37 mmol) of 18-crown-6 was added. The mixture was heated at reflux for 2.5 hours. After cooling to approx. 120° C., the mixture was filtered and the residue was washed repeatedly with chlorobenzene. After cooling to room temperature, 9.70 g (74%) of the product were obtained from the filtrate.

### Example I.b

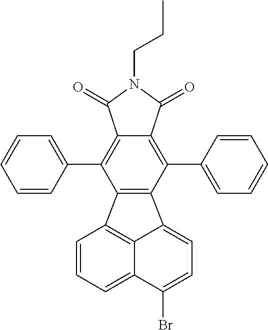

0.41 g (0.9 mmol) of the compound obtained in example I.a was suspended in 30 ml of glacial acetic acid. To this were added 0.46 ml (9 mmol) of bromine and one grain of iodine. The mixture was stirred at 30° C. for 28 hours. Then another 0.12 ml (2.25 mmol) of bromine was added and the mixture was stirred at room temperature for six hours. Subsequently, the bromine was expelled by bubbling, and the residue was filtered off and washed with water and ethanol. This gave 362 mg (75%) of the title compound.

### Example I.c

### N-Phenyl-4-bromonaphthalene-1,8-dicarboxylic monoimide

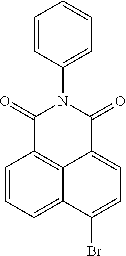

A mixture of 10.0 g (34 mmol) of 4-bromo-1,8-naphthalenedicarboxylic monoanhydride, 4.4 g (5.7 mmol) of aniline and 100 ml of propionic acid was heated to reflux overnight. After cooling to room temperature, the precipitate was filtered off with suction, washed with water and dried. This gave 10.1 g (68%) of the title compound as a colorless compound.

### Example I.d

### N-Phenyl-4-(pinacolatoboron)naphthalene-1,8-dicarboxylic monoimide

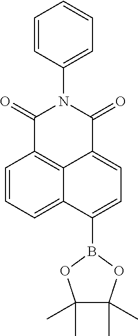

A mixture of 3.0 g (8.5 mmol) of the compound from example I.c, 3.2 g (12.8 mmol) of bispinacolatodiborane, 624 mg (0.8 mmol) of (1,1-bis(diphenylphosphino)ferrocene)-dichloropalladium, 2.5 g (25.6 mmol) of potassium acetate was heated in 100 ml of toluene to 70° C. overnight. The salts were filtered off and the filtrate, after removing the solvent, was purified by chromatography with 5:1 cyclohexane/ethyl acetate. This gave 2.5 g (73%) of the title compound as a beige solid.

### Example I.e

### N-(1′-Heptyloctyl)-4-bromonaphthalene-1,8-dicarboxylic monoimide

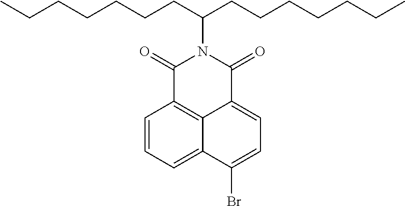

A mixture of 10.0 g (36 mmol) of 4-bromonaphthalenedicarboxylic monoimide, 14.0 g (43.3 mmol) of 1-heptyloctylamine and 5.3 g (29 mmol) of zinc acetate in 250 ml of quinoline was heated at reflux for five hours. After cooling to room temperature, the reaction mixture was poured onto 200 ml of 1M hydrochloric acid and then extracted with dichloromethane. The organic solvent was removed and the crude product was purified on silica gel using toluene. This gave 16.4 g (93%) of an oily product.

R_{f}(1:1 toluene:petroleum ether)=0.55

### Example I.f

### N-(1′-Heptyloctyl)-4-(pinacolatoboron)naphthalene-1,8-dicarboxylic monoimide

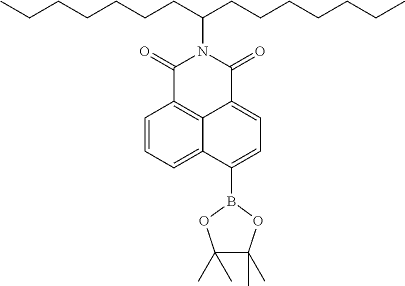

A mixture of 8.0 g (16.5 mmol) of the compound from example 1.e, 6.26 g (25 mmol) of bispinacolatodiborane, 1.2 g (1.6 mmol) of (1,1′-bis(diphenylphosphino)ferrocene)-dichloropalladium and 4.8 g (49 mmol) of potassium acetate in 200 ml of toluene was heated to 70° C. for 16 hours. After cooling to room temperature, insoluble constituents were filtered off and the product was purified on silica gel with 1:2 toluene/petroleum ether. This gave 6.4 g (73%) of a yellowish oil.

R_{f} (1:1 toluene:petroleum ether)=0.16

### Example I.g

Mixture of

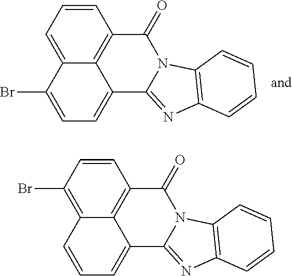

A mixture of 10.0 g (36 mmol) of 4-bromo-1,8-naphthalic acid anhydride, 4.68 g (43.3 mmol) of 1,2-phenylenediamine, 6.62 g of zinc(II) acetate (36 mmol) and 100 ml of quinoline were refluxed at 145° C. for 5 hours. The reaction mixture was poured onto 500 ml of 1M hydrochloric acid. The precipitate was sucked off, washed with hot water and then crystallized from toluene to give 10.6 g (84%) of the title compound as a yellow compound.

R_{f}(2:1 cyclohexane:ethyl acetate)=0.29

### Example I.h

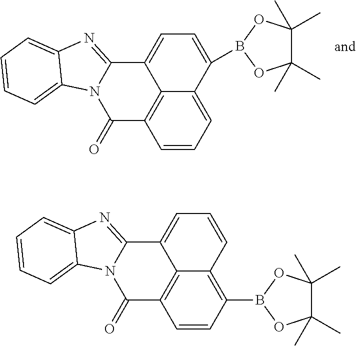

A mixture of 5.0 g (14.3 mmol) from example I.g, 10.9 g (43 mmol) of bis(pinacolato)diborone, 1.05 g of (1.43 mmol) of 1,1′-bis(diphenylphosphino)ferrocene-dichloropalladium, 4.22 g of potassium acetate (43 mmol) and 180 ml of toluene were stirred at 70° C. for 20 hours under nitrogen. The solvent was removed under reduced pressure. After cooling to room temperature, the product was purified on silica gel with dichloromethane and then crystallized from toluene/petrol ether. This gave 4.8 g (85%) of a yellow compound

R^{f} (dichloromethane)=0.35

### Example I.i

### 7,9-Diphenyl-6b,7-dihydro-cyclopenta[a]acenaphthylen-8-one

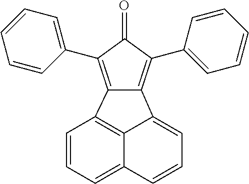

A suspension of 16.2 g (77 mmol) of 1,3-diphenyl-propan-2-one and 14.2 g of acenaphtenquinone in 8.2 ml of toluene and 82 ml of ethanol was refluxed at 78° C. under nitrogen. A solution of 1.35 g of KOH in 27 ml of ethanol was added dropwise over 15 minutes, and then refluxing was continued for an additional hour. The reaction mixture was cooled to 0-5° C. The black precipitate was sucked off, washed with ethanol (3×100 ml) and dried. This gave 26.2 g of the title compound (yield: 96%).

R_{f} (10:1 toluene:ethyl acetate): 0.8

### Example I.k

### 7,11-Diphenyl-9-propyl-9-aza-cyclopenta[k]fluoranthene-8,10-dione

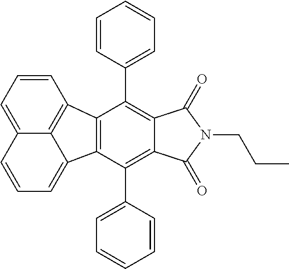

A mixture of 5.0 g (14 mmol) of 7,9-diphenyl-6b,7-dihydro-cyclopenta[a]acenaphthylen-8-one from example I.i, 2.93 g (21.5 mmol) of N-propylmaleimide and 70 ml of chlorobenzene was refluxed at 132° C. for 3 hours and then cooled to 70° C. To this mixture 2.9 g (18.25 mmol) of potassium permanganate and 4.9 g (18.25 mmol) of 18-crown-6 were added. The reaction mixture was stirred for another 2.5 hours under reflux, filtered while still hot and washed with chlorobenzene (3×10 ml). After standing overnight at room temperature, the precipitate thus formed was sucked off. This gave 3.1 g (yield: 47.5%) of a yellow compound.

R_{f} (10:1 toluene:petroleum ether)=0.11

### Example I.l

### 3-Bromo-7,11-diphenyl-9-propyl-9-aza-cyclopenta[k]fluoranthene-8,10-dione

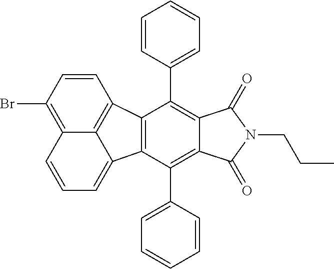

To a suspension of 2.5 g (5.37 mmol) of 7,11-diphenyl-9-propyl-9-aza-cyclopenta[k]fluoranthene-8,10-dione from example I.k, a small amount of iodine and 150 ml of glacial acetic acid and 5.5 ml (110 mmol) of bromine were added dropwise. The mixture was stirred at 30° C. for another 20 hours. Excess bromine was removed by bubbling nitrogen through the reaction mixture followed by the addition of 500 ml of a sodium thiosulphate solution (1% by weight). The reaction mixture was stirred for 30 minutes. The precipitate was sucked off and washed with water. The title compound was crystallized from toluene: petroleum ether. This gave 3.1 g (100% yield) of the title compound as a yellow compound.

R_{f} (dichloromethane): 0.42

### Example I.m

### 3-Pinacolatoborylo-7,11-diphenyl-9-propyl-9-aza-cyclopenta[k]fluoranthene-8,10-dione

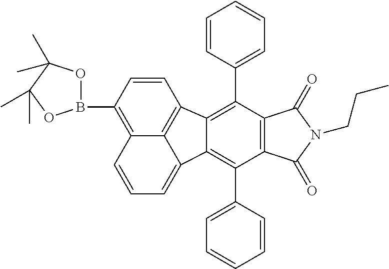

A mixture of 3.0 g (5.51 mmol) of the compound from example I.l, 4.0 g (16.5 mmol) of bispinacolatodiborane, 0.41 g (0.55 mmol) of (1,1′-bis(diphenylphosphino)ferrocene)-dichloropalladium and 1.62 g (43 mmol) of potassium acetate in 70 ml of toluene was heated to 70° C. for 20 hours. The solvent was removed under reduced pressure. The product was purified on silica gel with dichloromethane and crystallized from toluene/petroleum ether. This gave 2.5 g (76%) of the title compound as yellow compound.

R_{f}(dichloromethane)=0.34

### Example I.n

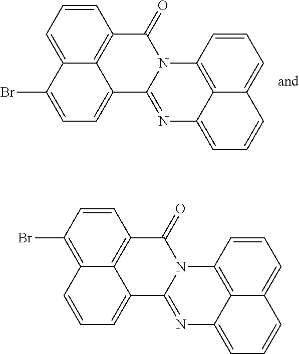

A mixture of 3.0 g (10.8 mmol) 4-bromo-1,8-naphthalic acid anhydride, 2.1 g (13.0 mmol) of 1,8-diaminonaphthalene, 1.99 g of zinc(II) acetate (10.8 mmol) and 300 ml of quinoline were refluxed at 145° C. for 5 hours. The reaction mixture was poured onto 500 mL of 1M hydrochloric acid. The precipitate was sucked off, washed with hot water and then crystallized from toluene to give 3.2 g (74%) of a violet-red compound.

R_{f} (dichloromethane)=0.80

### Example I.1

### 3-Bromo-7,14-diphenylbenzofluoranthene

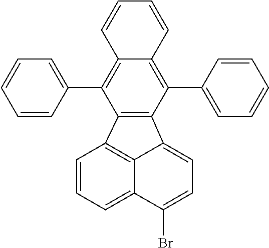

A mixture of 50 ml of N-methylpyrrolidone (NMP), 500 ml of dichloromethane, 10.0 g (25 mmol) of 7,14-diphenylbenzofluoranthene (prepared as described in WO 2010/031833) and 6.45 g (36 mmol) of N-bromosuccinimide was stirred at room temperature for 20 hours. Subsequently, the dichloromethane was removed under reduced pressure, and the product was precipitated by adding water, filtered off and dried. This gave 12.1 g (quant.) of the title compound as a beige solid.

R_{f} (2:1 petroleum ether:toluene)=0.9

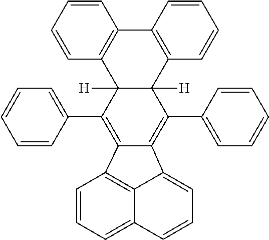

### Example I.2

A solution of 15 g (40 mmol) of phencyclone and 7.6 g (40 mmol) of acenaphthylene in 300 ml of xylene was heated at reflux overnight. After cooling, the title compound was precipitated by adding ethanol. This gave 20.6 g (quant) of a white solid.

R_{f} (5:1 toluene:petroleum ether)=0.5

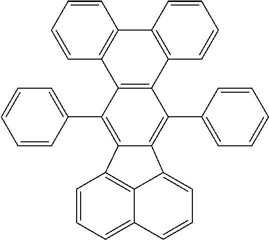

### Example I.3

A mixture of 4.5 g (8.9 mmol) of the compound from example I.2, 13.0 g (57 mmol) of DDQ in 1500 ml of chlorobenzene was heated to 134° C. for 60 hours. A solution of sodium thiosulfate was added, the chlorobenzene was distilled off and the mixture was admixed with dichloromethane, which gave a solid. The chromatographic purification of the crude product on silica gel with 10:1 petroleum ether/toluene gave 1.4 g (31%) of the title compound.

R_{f} (1:5 toluene:petroleum ether)=0.32

### Example I.4

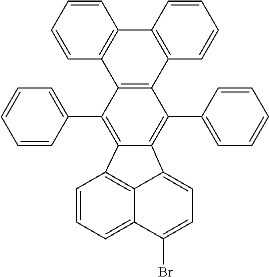

A mixture of 100 mg (0.2 mmol) of the compound from example I.3, 0.3 g (2 mmol) of bromine and 5 ml of glacial acetic acid was stirred at room temperature for two hours. Subsequently, the bromine was expelled by bubbling with N2 and the product was isolated by filtration.

R^{f}(1:5 toluene:petroleum ether)=0.59

### Example I.5

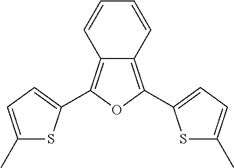

To 63 ml of a 1M solution of 2-methyl-thiophenyl magnesium bromide in diethyl ether 4.92 g (30 mmol) of 3-methoxy-3H-isobenzofuran-1-one in 50 ml of diethyl ether was added dropwise for 1 h at −5° C. while stirring under protective atmosphere. The suspension was stirred for another hour at this temperature and then overnight at room temperature. The suspension was cooled to −5° C. The product was treated with ice water and then 1 M HCl was added. The reaction mixture was extracted with dichloromethane and the organic phase was dried over sodium sulfate. The product was used in solution without further purification for the next step.

R_{f} (10:1 toluene: ethyl acetate)=0.82

### Example I.6

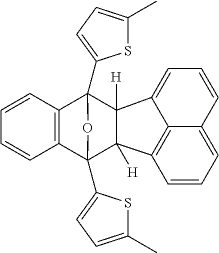

To a solution of 4.46 g (29.3 mmol) of acenaphthylene in 150 ml of o-xylene 9.1 g (29.33 mmol) of the compound from example I.5 in 700 ml of dichloromethane and diethyl ether was added at 100° C. The low boilers were then separated and the mixture was refluxed (140° C.) for another 2 hours while stirring. The product was poured slowly into 1 l of petroleum ether and the mixture was stirred for another 2 hours. The formed precipitate was filtered off and the filtrate was concentrated. This gave 16.33 g of the title compound (yield: 100%).

R_{f} (100:1 toluene:acetone)=0.53

### Example I.7

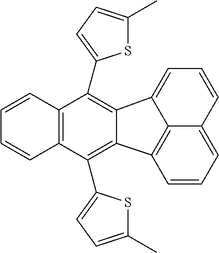

To a solution of 13.55 g (29.3 mmol) of the compound from example 1.6 in 100 ml of acetic acid 14.83 g (88 mmol) of 48% HBr was added. The mixture was refluxed (ca. 110° C.) for 4 hours. The reaction mixture was poured into water and then extracted with dichloromethane and toluene. The organic phase was dried and concentrated. This gave 14.7 g of the crude product. The blue fluorescent product was isolated by thin layer chromatography (TLC).

R_{f} (1:2 toluene: petroleum ether)=0.6

### II. Preparation of Compounds I

### Example 1

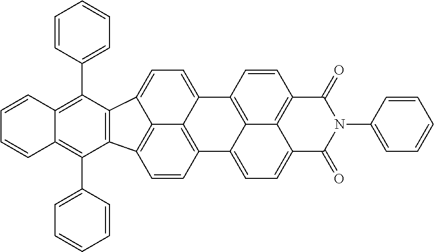

### Example 1.1

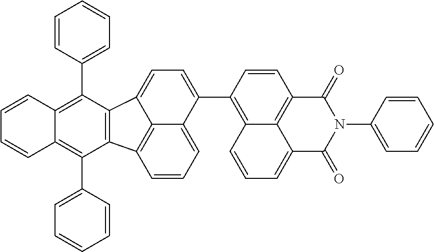

A mixture of 150 ml of toluene, 2.0 g (5 mmol) of the compound from example I.d (N-phenyl-4-(pinacolatoboron)napthalene-1,8-dicarboxylic monoimide), 1.44 g (3 mmol) of the compound from example I.1 (3-bromo-7,14-diphenylbenzofluoranthene), 4.4 g of potassium carbonate, 120 ml of water and 12 ml of ethanol, and also 72 mg (0.4 mmol) of tetrakistriphenylphosphinepalladium, was stirred at room temperature for 18 hours. Thereafter, a further 0.5 g (12.5 mmol) of the compound from example I.d was added and the mixture was stirred at room temperature for a further three hours. Subsequently, the phases were separated, the organic phase was washed and dried, and the residue was purified by chromatography using toluene. This gave 1.47 g (73%) of a yellow oil.

R_{f} (10:1 toluene:ethyl acetate)=0.52

### Example 1.2

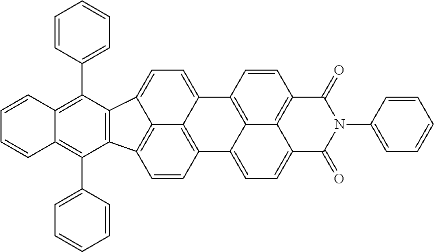

A mixture of 1.47 g (22 mmol) of the compound from example 1.1 and 4.3 g (33 mmol) of aluminum trichloride in 250 ml of chlorobenzene was heated to 120° C. for 18 hours. After cooling to room temperature, the aluminum chloride was hydrolyzed cautiously with water. The phases were separated and dried, and the solvent was removed under reduced pressure. The product was purified by chromatography with toluene. This gave a solid which was blue in the solid state and fluoresced red in solution.

R_{f} (10:1 toluene/ethyl acetate)=0.35.

### Example 2

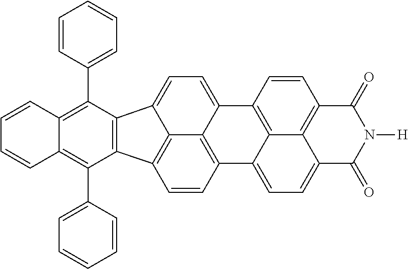

### Example 2.1

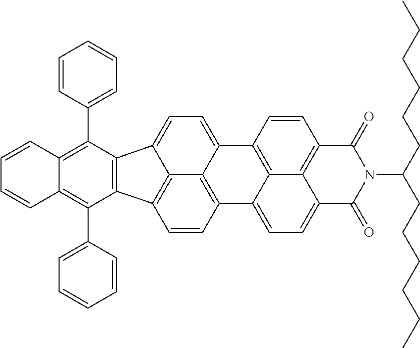

A mixture of 20 ml of toluene, 0.483 g (1 mmol) of the compound from example I.1 (3-bromo-7,14-diphenylbenzofluoranthene) and 1.0 g (1.24 mmol) of the borane compound from example 1.f, 3.0 g of potassium carbonate, 20 ml of water, 8 ml of ethanol and 0.5 g (0.04 mmol) of tetrakistriphenylphosphinepalladium was heated to 40° C. for 2 hours. The solvents were removed under reduced pressure and the residue was purified by chromatography on silica gel with toluene/petroleum ether mixtures. This gave 0.7 g (86%) of a yellow compound.

R_{f} (1:2 toluene:petroleum ether)=0.32

### Example 2.2

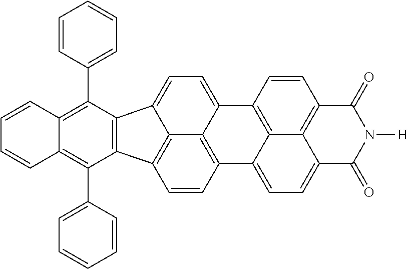

A mixture of 70 ml of chlorobenzene, 0.7 g (0.9 mmol) of the compound from example 2.1 and 2.13 g (13 mmol) of aluminum trichloride was heated to 120° C. for 16 hours. The solvent was removed under reduced pressure, taken up with dichloromethane, filtered and washed with water. This gave 0.39 g (56%) of the title compound as a blue solid.

### Example 3

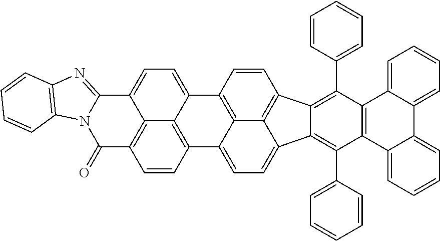

### Example 3.1

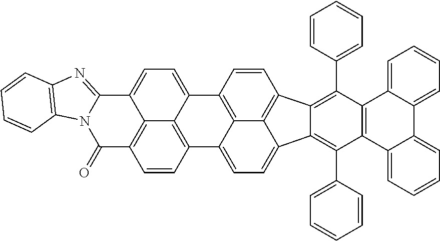

A mixture of 100 ml of toluene, 2.00 g (3.43 mmol) of the compound from example 1.4, 2.04 g (5.14 mmol) of the borane compound from example 1.h, 12.5 g of potassium carbonate, 50 ml of water, 20 ml of ethanol and 1.98 g (1.71 mmol) of tetrakistriphenylphosphinepalladium was heated to 70° C. for 16 hours. The organic phase was dried over magnesium sulfate and purified by chromatography on silica gel with toluene. This gave 1.5 g (56%) of a yellow compound. R^{f} (dichloromethane)=0.37

### Example 3.2

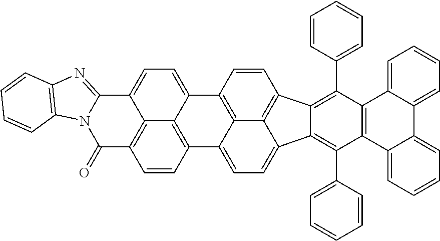

A mixture of 15.8 g (104 mmol) diazabicyclo[5.4.0]undec-7ene, 15.8 g (259 mmol) of ethanolamine and 10.5 g (93.2 mmol) of potassium tert-butoxide was heated to 120° C. for 30 minutes. To this mixture, 1.00 g (1.3 mmol) of the compound from example 3.1 was added at 50° C. The reaction mixture was heated to 160° C. for another 48 hours while stirring. The crude product was precipitated by pouring in diluted hydrochloric acid and the filtration residue was mixed with dichloromethane several times. This gave 410 mg (40%) of the title compound as violet solid.

R_{f} (1:5 trichloroacetic acid: toluene)=0.42

### Example 4

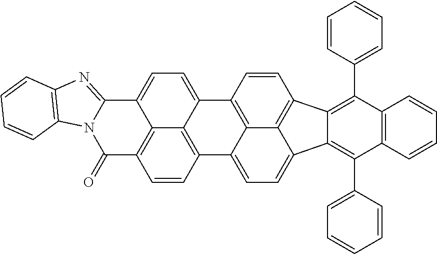

### Example 4.1

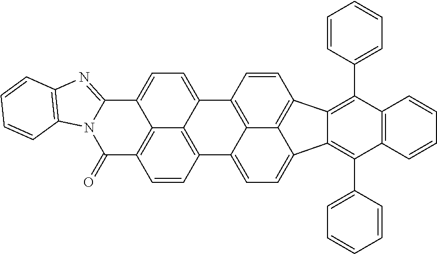

A mixture of 160 ml of toluene, 1.76 g (3.64 mmol) of the compound from example I.1, 1.60 g (4.00 mmol) of the borane compound from example 1.h, 13.1 g of potassium carbonate, 80 ml of water, 32 ml of ethanol and 2.1 g (1.82 mmol) of tetrakistriphenylphosphinepalladium was heated to 65° C. for 16 hours. The organic phase was separated off, dried over magnesium sulfate and purified by chromatography on silica gel with toluene. This gave 2.0 g (82%) of a yellow compound.

R^{f} (dichloromethane)=0.30

### Example 4.2

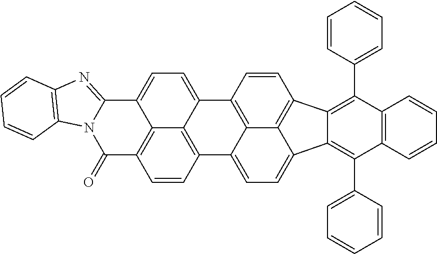

A mixture of 9.00 g (60 mmol) diazabicyclo[5.4.0]undec-7ene, 9.0 g (150 mmol) of ethanolamine and 6.00 g (54 mmol) of potassium tert-butoxide was heated to 120° C. for 30 minutes. To this mixture, 500 mg (0.74 mmol) of the compound from example 4.1 was added at 50° C. The reaction mixture was heated to 160° C. for another 48 hours while stirring. The crude product was precipitated by pouring in diluted hydrochloric acid and the filtration residue was mixed with dichloromethane several times. This gave 170 mg (33%) of the title compound as blue compound.

R_{f} (1:5 trichloroacetic acid: toluene)=0.3

### Example 5

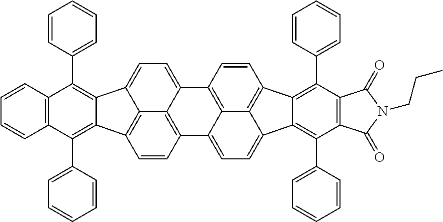

### Example 5.1

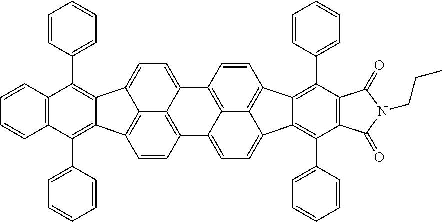

A mixture of 66 ml of toluene, 1.00 g (2.07 mmol) of the compound from example I.1, 1.35 g (2.28 mmol) of the borane compound from example 1.m, 7.43 g (54 mmol) of potassium carbonate, 44 ml of water, 18 ml of ethanol and 2.0 g (1.03 mmol) of tetrakistriphenylphosphinepalladium was heated to 70° C. for 16 hours. The organic phase was separated off, dried over magnesium sulfate and purified by chromatography on silica gel with toluene. This gave 0.67 g (37%) of a yellow compound.

R^{f} (dichloromethane)=0.55

### Example 5.2

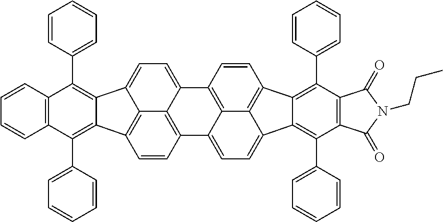

A mixture of 50 ml of chlorobenzene, 0.5 g (0.58 mmol) of the compound from example 4.1 and 1.83 g (13 mmol) of aluminum trichloride was heated to 110° C. for 3 hours. 100 ml of dichloromethane was added and the reaction mixture was extracted with water. The organic phase was purified by chromatography on silica gel with dichloromethane, This gave 100 mg (20%) of the title compound as a blue solid.

### III. Production of the Cells

### III.1 Production of the Cells for the Compound of Example 1 and Results:

Substrate: ITO was sputtered onto the glass substrate in a thickness of 100 nm. The specific resistivity was 200μΩ cm, and the mean roughness (RMS; roughness mean square) was less than 5 nm. Before the deposition of the further layers, the substrate was treated with ozone under UV light for 20 minutes (UV-ozone cleaning).

Bilayer cells (cells of two-layer construction) and bulk heterojunction cells (BHJ cells) were produced under high vacuum (pressure <10⁻⁶ mbar).

Bilayer Cell (ITO/MoO₃/Inventive Compound of the Formula I/C₆₀/Bphen/Ag):

The bilayer cell was produced by successive deposition of an inventive compound of the formula I and C₆₀ (layer thickness 40 nm) onto MoO₃-coated ITO substrate. The deposition rate for both layers was 0.1 nm/second. The evaporation temperature of the inventive compound of the formula (I) is reproduced in table 1 below.

TABLE 1

| Inventive compound | Evaporation temperature |
|---|---|
| of the formula (I) | [° C.] |
| Example 1 | 340 |

C₆₀ was deposited at 410° C. Once the Bphen layer (layer thickness 6 nm) had been applied, a 100 nm thick Ag layer was finally applied by vapor deposition as the top electrode. The cell had an area of 0.04 cm².

BHJ Cell (ITO/MoO₃/(Inventive Compound of the Formula (I):C₆₀)/C₆₀/Bphen/Ag):

To produce the BHJ cell (bulk heterojunction cell), an inventive compound of the formula (I) and C₆₀ were coevaporated. The Bphen and Ag layers were deposited as described for the bilayer cell. The layer thicknesses were 6 nm for BPhen and 100 nm for Ag.

### Tests:

The solar simulator used was an AM 1.5 Simulator from Solar Light Co. Inc. with a xenon lamp (model 16S-150 V3). The UV range below 380 nm was filtered and the current-voltage measurements were made under ambient conditions. The intensity of the solar simulator was calibrated with a monocrystalline FZ solar cell (FRAUNHOFER ISE), and the deviation factor was determined to be approximately 1.0.

### Bilayer Cell:

TABLE 2

| | Layer | | | | |
|---|---|---|---|---|---|
| | thickness | Voc | I_{SC} | FF | η |
| Compound | [nm] | [mV] | [mA/cm²] | [%] | [%] |
| Example 1 | 10 | 1110 | 2.5 | 54 | 1.5 |

### BHJ Cell:

TABLE 3

| | | Weight ratio | | | | |
|---|---|---|---|---|---|---|
| | Layer | (inventive | | | | |
| | thickness | compound | V_{OC} | I_{SC} | FF | η |
| Compound | [nm] | I:C60) | [mV] | [mA/cm²] | [%] | [%] |
| Example 1 | 40 | 1:1 | 1120 | 5.9 | 41 | 2.7 |
| | 45 | 1:1.5 | 1120 | 5.9 | 40 | 2.7 |
| | 30 | 1:2 | 1100 | 5.7 | 40 | 2.5 |

η efficiency
FF fill factor
I_{sc} short-circuit current
V_{oc} open-circuit voltage

### III.2 Production of the Cells for the Compound of Examples 3 and 4

### Materials:

C60: obtained from CreaPhys;
Bphen: obtained from Fluka;
Compound from example 3 and example 4, respectively;
MoO₃: obtained from Merck

ITO was sputtered onto the glass substrate in a thickness of 120 nm. The specific resistivity was 15Ω cm, and the mean roughness (RMS; roughness mean square) was less than 2 nm. Before the deposition of the further layers, the substrate was treated with ozone under UV light for 15 minutes (UV-ozone cleaning).

Bilayer cells (cells of two-layer construction) and bulk heterojunction cells (BHJ cells) were produced under high vacuum (pressure: ca 2×10⁻⁶ mbar).

Bilayer Cell (ITO/MoO₃/Inventive Compound of the Formula I/C₆₀/Bphen/Ag):

The bilayer cell was produced by successive deposition of an inventive compound of the formula I and C₆₀ onto MoO₃-coated ITO substrate. The deposition rate for both layers was 0.3 nm/second. 060 was evaporated at 340° C. (compound from example 3) and 380° C. (compound from example 4), respectively. The evaporation temperature of the inventive compound of the formula (I) is reproduced in table 4 below. Bphen was evaporated at 150° C. (compound from example 3) and 130° C. (compound from example 4), respectively. Finally 100 nm of Ag was evaporated for the top contact. The device had an area of 0.04 cm². The cell structure of the bilayer cell is shown in table 5. The compound of the formula I is either the compound of example 3 or of example 4

TABLE 4

| Inventive compound | Evaporation temperature |
|---|---|
| of the formula (I) | [° C.] |
| Example 3 | 380 |
| Example 4 | 350 |

TABLE 5
cell structur of the bilayer cell

| | | compound of | | |
|---|---|---|---|---|
| ITO | MoO₃ [nm] | formula (I) [nm] | C60 [nm] | Bphen/Ag [nm] |
| | 5 | 10 | 35 | 5/100 |
| | | 20 | | |

### BHJ Cell (ITO/MoO₃/(Inventive Compound of the Formula (I):C₆₀)/C₆₀/Bphen/Ag):

To produce the BHJ cell (bulk heterojunction cell), an inventive compound of the formula (I) and C₆₀ were coevaporated. The inventive compound of the formula (I) to C₆₀ weight ratio was 1:1.5. The Bphen and Ag layers were deposited as described for the bilayer cell. The layer thicknesses were 5 nm for BPhen and 100 nm for Ag. The cell structure of the BHJ cell is shown in table 6. The compound of the formula I is either the compound of example 3 or of example 4.

TABLE 6
cell structure of the BHJ cell

| | MoO₃ | thickness: compound of the | C60 | Bphen/Ag |
|---|---|---|---|---|
| ITO | [nm] | formula (I) + C₆₀ [nm] | [nm] | [nm] |
| | 5 | 15 | 25 | 5/100 |
| | | 30 | | |
| | | 45 | | |
| | | 60 | | |

### Measurements:

The solar simulator used was an AM 1.5 Simulator from Solar Light Co. Inc. with a xenon lamp (model 16S-150 V3). The UV range below 380 nm was filtered and the current-voltage measurements were made under ambient conditions. The intensity of the solar simulator was calibrated with a monocrystalline FZ solar cell (Fraunhofer ISE), and the deviation factor was determined to be approximately 1.0.

### Results:

TABLE 7
bilayer cell: ITO/MoO₃/compound of formula I/C60/Bphen/Ag)

| | thickness | η | FF | I_{SC} | Voc |
|---|---|---|---|---|---|
| compound | [nm] | [%] | [%] | [mA/cm²] | [mV] |
| example 3 | 10 | 0.24 | 29 | −1.7 | 500 |
| | 20 | 0.04 | 21 | −0.4 | 640 |
| example 4 | 10 | 1.04 | 41 | −2.6 | 1000 |
| | 20 | 0.3 | 15 | −1.8 | 1000 |

TABLE 8
BHJ cell: (ITO/MoO₃/(compound of formula I:C60)/C60/Bphen/Ag)

| | thickness | η | FF | I_{SC} | Voc |
|---|---|---|---|---|---|
| compound | [nm] | [%] | [%] | [mA/cm²] | [mV] |
| example 3 | 15 | 0.3 | 37 | −1.9 | 500 |
| | 30 | 0.3 | 39 | −1.3 | 480 |
| | 45 | 0.2 | 37 | −0.9 | 480 |
| | 60 | 0.1 | 36 | −0.6 | 460 |
| example 4 | 15 | 1.6 | 50 | −4.3 | 820 |
| | 30 | 1.5 | 37 | −5.0 | 840 |
| | 45 | 1.2 | 35 | −4.2 | 840 |
| | 60 | 0.8 | 33 | −3.1 | 820 |

1. An organic solar cell with a photoactive region which comprises at least one organic donor material in contact with at least one organic acceptor material, wherein the donor material and the acceptor material form a donor-acceptor heterojunction and wherein the photoactive region comprises at least one substituted perylene of the formula (I) 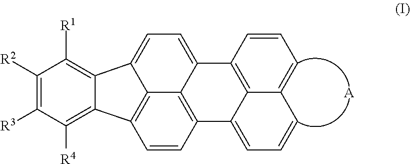
in which
R¹ and R⁴ are independently selected from hydrogen and in each case unsubstituted or substituted alkyl, aryl, heteroaryl or oligo(het)aryl,
R² and R³ are independently selected from hydrogen and in each case unsubstituted or substituted alkyl, aryl, heteroaryl or oligo(het)aryl,
where in each case at least two adjacent radicals, selected from the R¹, R², R³ and R⁴ radicals, together with the carbon atoms of the benzene ring to which they are bonded, may also be a fused ring system having 1, 2, 3, 4, 5, 6, 7 or 8 further rings, and
A together with the carbon atoms to which it is bonded is a fused monocyclic, dicyclic, tricyclic, tetracyclic, pentacyclic or hexacyclic ring system having at least one exocyclic keto group, where the ring system optionally bears one or more substituents bonded via a single bond.

2. The organic solar cell according to claim 1, wherein R¹, R², R³ and R⁴ are each independently selected from hydrogen, unsubstituted alkyl, aralkyl, unsubstituted aryl, alkaryl, halogen-substituted aryl, heteroaryl or oligo(het)aryl.

3. The organic solar cell according to either of claims 1 and 2, wherein R¹ and R⁴ are each independently selected from hydrogen and groups of the general formulae (III.1) to (III.12) 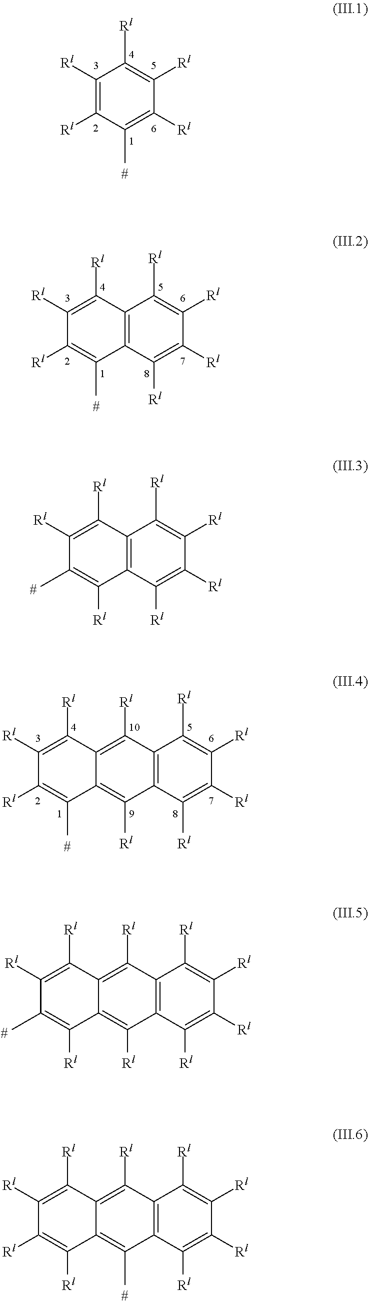 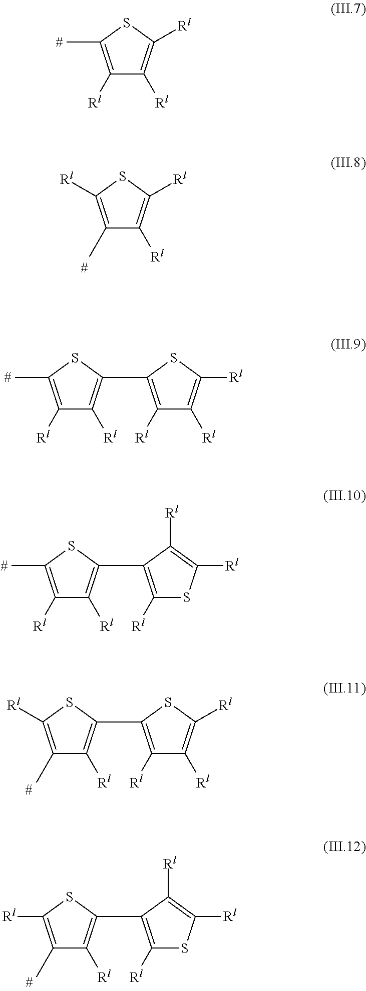
in which
# is the bonding site to the benzene ring, and
R^{I} are each independently selected from hydrogen, fluorine, chlorine, unsubstituted alkyl and substituted alkyl.

4. The organic solar cell according to any of the preceding claims, wherein R¹ and R⁴ are each independently selected from hydrogen and groups of the general formulae (III.1a), (III.1b), (III.1c), (III.1d), (III.2a), (III.4a), (III.7a), (III.7b), (III.8a), (III.8b), (III.9a), (III.9b), (III.9c), (III.9d), (III.10a), (III.10b), (III.10c), (III.10d), (III.11a), (III.11b), (III.11c), (III.11d), (III.12a), (III.12b), (III.12c) and (III.12d): 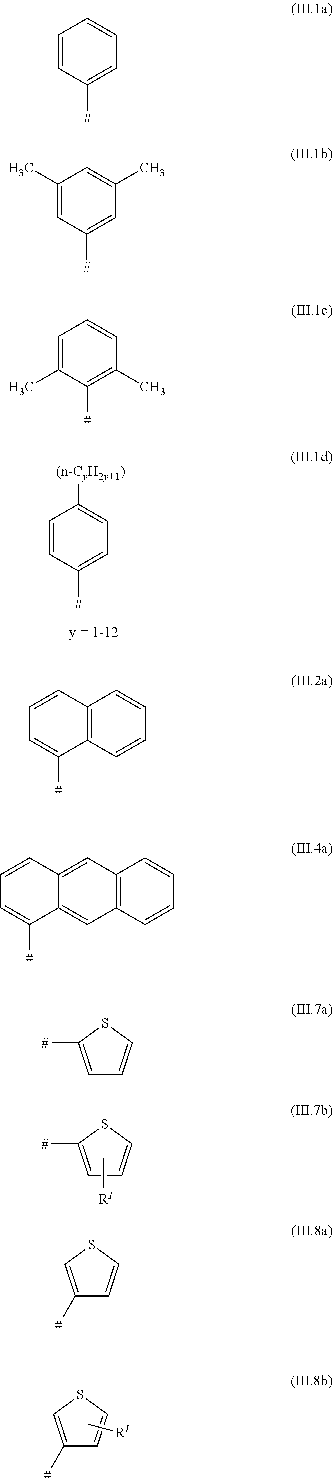 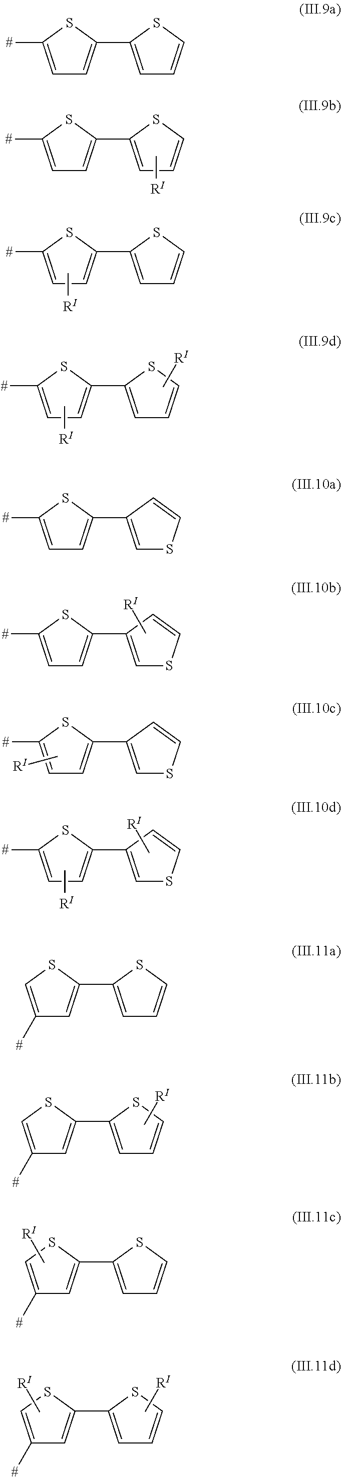 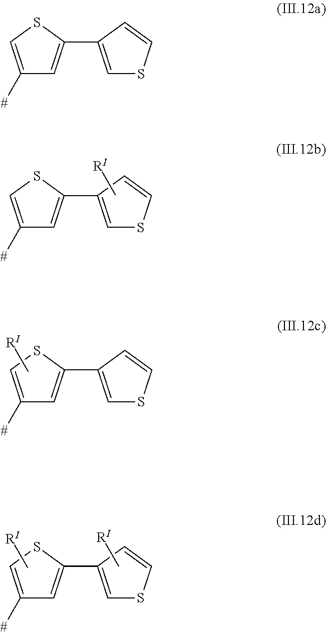
in which
# is the bonding site to the benzene ring; and
R^{I} is C₁-C₈-alkyl.

5. The organic solar cell according to any of the preceding claims, wherein the R¹ and R⁴ groups are each independently selected from hydrogen and phenyl.

6. The organic solar cell according to any of the preceding claims, wherein the R² and R³ groups in the compounds of the general formula (I) are each independently selected from hydrogen and groups of the general formulae (IV.1) to (IV.12) 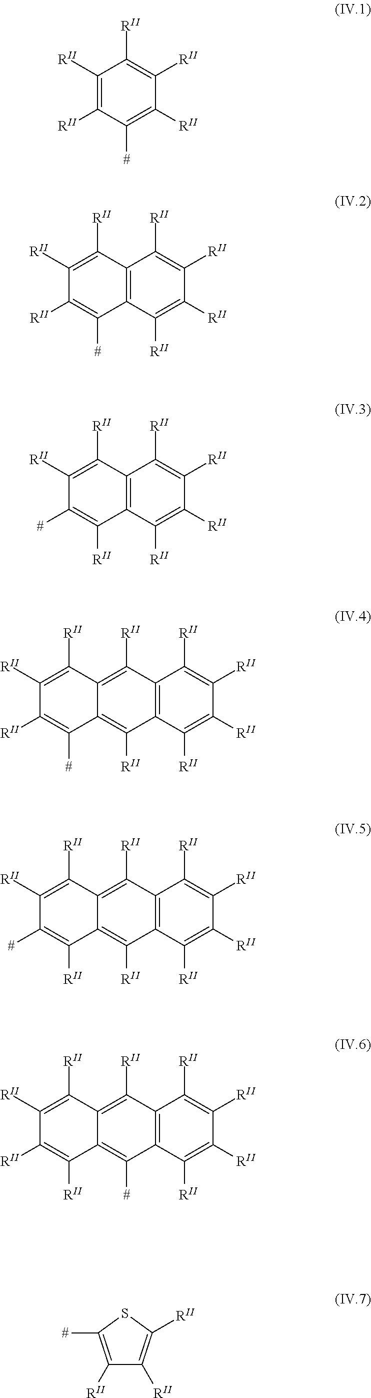 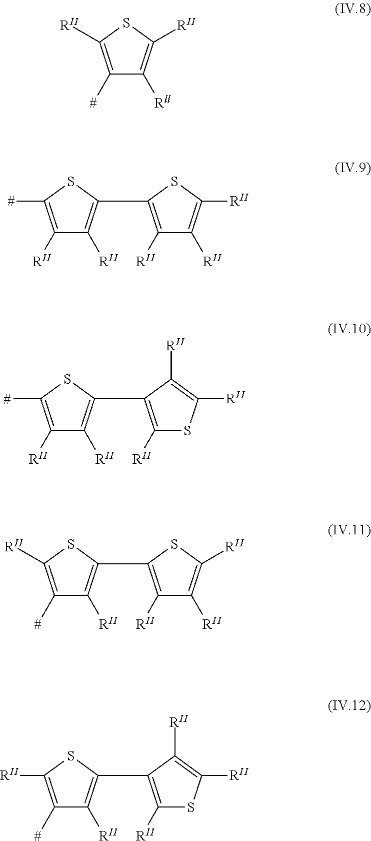
in which
# is the bonding site to the benzene ring, and
R^{II} are each independently selected from hydrogen, fluorine, chlorine, unsubstituted alkyl and substituted alkyl.

7. The organic solar cell according to any of the preceding claims, wherein R² and R³ are each independently selected from hydrogen and groups of the general formulae (IV.1a), (IV.1b), (IV.1c), (IV.1d), (IV.2a), (IV.4a), (IV.7a), (IV.7b), (IV.8a), (IV.8b), (IV.9a), (IV.9b), (IV.9c), (IV.9d), (IV.10a), (IV.10b), (IV.10c), (IV.10d), (IV.11a), (IV.11b), (IV.11c), (IV.11d), (IV.12a), (IV.12b), (IV.12c) and (IV.12d): 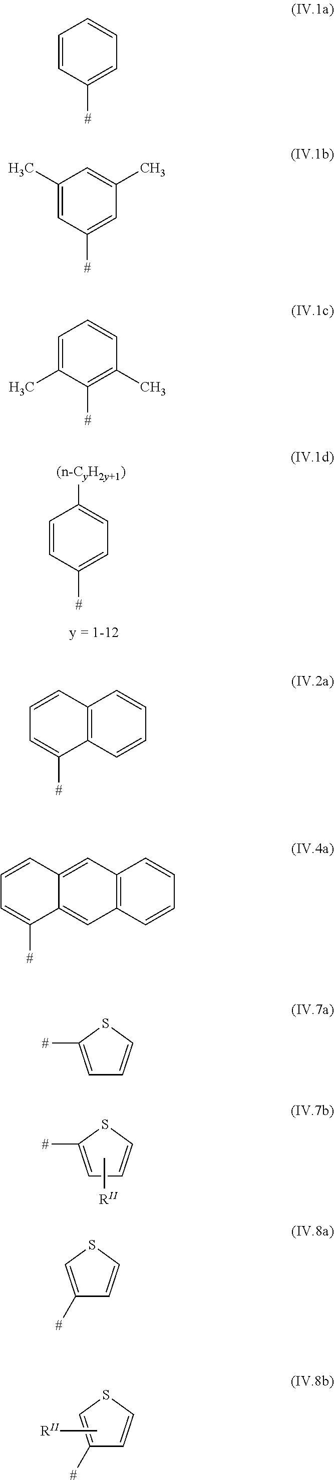 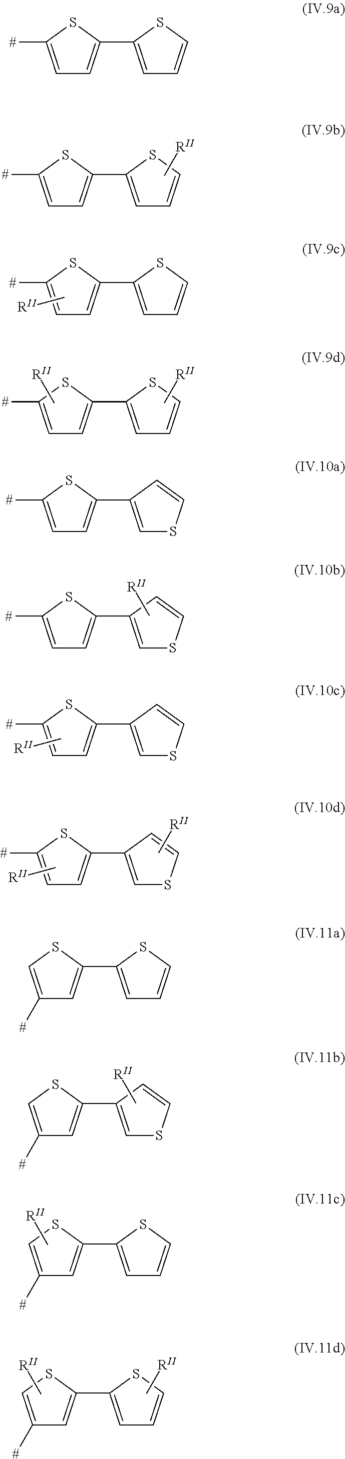 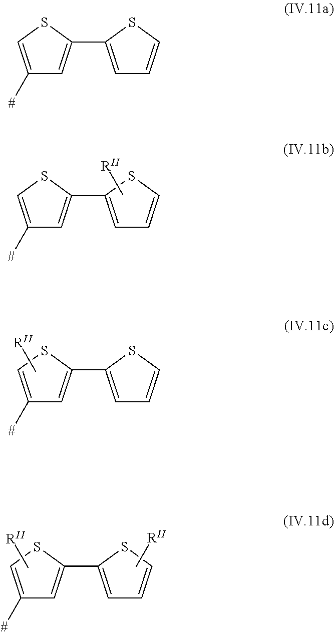
in which
# is the bonding site to the benzene ring; and
R^{II} is C₁-C₈ alkyl.

8. The organic solar cell according to any of the preceding claims, wherein the R² and R³ groups are each independently selected from hydrogen and phenyl.

9. The organic solar cell according to any of claims 1 to 5, wherein the R² and R³ groups together are a group which is selected from groups of the general formulae (V.1), (V.2) and (V.3) 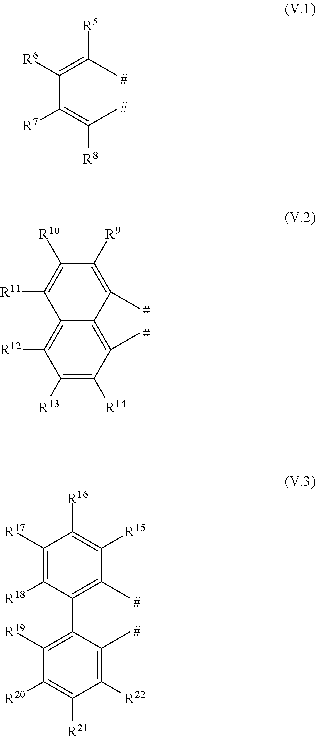
in which
# in each case is the bonding site to the benzene ring;
R⁵, R⁶, R⁷ and R⁸ are each independently hydrogen, C₁-C₂₀-alkyl, halogen or phenyl, where phenyl may be unsubstituted or may bear 1, 2 or 3 R^{a} radicals;
R⁹, R¹⁰, R¹¹, R¹², R¹³ and R¹⁴ are each independently hydrogen, C₁-C₂₀-alkyl, halogen or phenyl, where phenyl may be unsubstituted or may bear 1, 2 or 3 R^{a} radicals; and
R¹⁵, R¹⁶, R¹⁷, R¹⁸, R¹⁹, R²⁰, R²¹ and R²² are each independently hydrogen, C₁-C₂₀-alkyl, halogen or phenyl, where phenyl may be unsubstituted or may bear 1, 2 or 3 R^{a} radicals, in which
R^{a} is C₁-C₁₀-alkyl or C₁-C₆-alkoxy.

10. The organic solar cell according to claim 9, wherein the R² and R³ groups together are a group of the formula V.1.

11. The organic solar cell according to claim 10, wherein R⁵, R⁶, R⁷ and R⁸ are each hydrogen.

12. The organic solar cell according to any of the preceding claims, in which A is selected from groups of the general formulae (VI.1), (VI.2), (VI.3), (VI.4) and (VI.5) 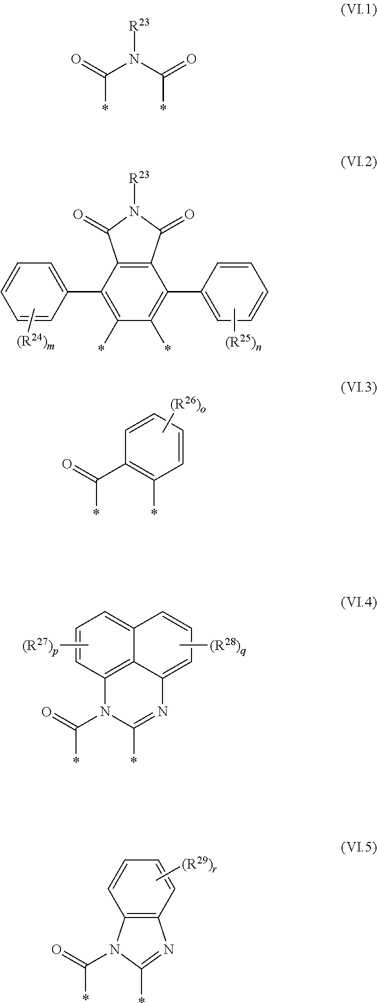
in which
* in each case is the bonding site to the perylene base skeleton,
R²³ is hydrogen, and in each case unsubstituted or substituted alkyl, cycloalkyl, heterocyclyl, aryl or hetaryl,
R²⁴, R²⁵ are each independently halogen, unsubstituted alkyl, substituted alkyl, unsubstituted aryl or substituted aryl, and where two R²⁴ and/or R²⁵ radicals bonded to adjacent carbon atoms may also be a fused-on benzene ring,
R²⁶ is halogen, unsubstituted alkyl, substituted alkyl, unsubstituted aryl or substituted aryl, and where two R²⁶ radicals bonded to adjacent carbon atoms may be a fused-on benzene ring,
R²⁷, R²⁸ are each independently halogen, unsubstituted alkyl, substituted alkyl, unsubstituted aryl or substituted aryl, and where two R²⁷ and/or R²⁸ radicals bonded to adjacent carbon atoms may also be a fused-on benzene ring,
R²⁹ is halogen, unsubstituted alkyl, substituted alkyl, unsubstituted aryl or substituted aryl, and where two R²⁹ radicals bonded to adjacent carbon atoms may be a fused-on benzene ring,
m, n are each independently 0, 1, 2 or 3,
o, p, q, r are each 0, 1 or 2.

13. The organic solar cell according to claim 12, in which, in the groups of the general formulae (VI.1), (VI.2), (VI.3), (VI.4) and (VI.5)
* in each case is the bonding site to the perylene base skeleton,
R²³ is hydrogen, C₁-C₂₀-alkyl, C₃-C₇-cycloalkyl, C₃-C₇-heterocyclyl, C₆-C₁₄-aryl-C₁-C₂₀-alkyl, C₆-C₁₄-aryl, C₁-C₂₀-alkyl-C₆-C₁₄-aryl, or a 5- or 6-membered heteroaromatic radical which has 1, 2 or 3 heteroatoms selected from O, S and N as ring members, and in which the heteroaromatic radical may have a fused-on benzene ring or naphthalene ring and may have 1, 2, 3, 4, 5 or 6 R^{b} substituents, and the aryl group in C₆-C₁₄-aryl-C₁-C₂₀-alkyl, C₆-C₁₄-aryl, C₁-C₂₀-alkyl-C₆-C₁₄-aryl may have 1, 2, 3, 4, 5 or 6 R^{b} substituents,
R²⁴, R²⁵ are each independently C₁-C₂₀-alkyl, halogen or phenyl, where phenyl may be unsubstituted or may bear 1, 2 or 3 R^{c} radicals, and where two R²⁴ and/or R²⁵ radicals bonded to adjacent carbon atoms may also be a fused-on benzene ring,
R²⁶ is C₁-C₂₀-alkyl, halogen or phenyl, where phenyl may be unsubstituted or may bear 1, 2 or 3 R^{c} radicals, and where two R²⁶ radicals bonded to adjacent carbon atoms may be a fused-on benzene ring,
R²⁷, R²⁸ are each independently C₁-C₂₀-alkyl, halogen or phenyl, where phenyl may be unsubstituted or may bear 1, 2 or 3 R^{c} radicals, and where two R²⁷ and/or R²⁸ radicals bonded to adjacent carbon atoms may also be a fused-on benzene ring,
R²⁹ is C₁-C₂₀-alkyl, halogen or phenyl, where phenyl may be unsubstituted or may bear 1, 2 or 3 R^{b} radicals, and where two R²⁹ radicals bonded to adjacent carbon atoms may be a fused-on benzene ring,
m, n are each independently 0, 1, 2 or 3,
o, p, q, r are each 0, 1 or 2,
R^{b} is fluorine, chlorine, cyano, nitro or C₁-C₂₀-alkoxy, and
R^{c} is fluorine, chlorine, cyano, nitro, C₁-C₂₀-alkyl or C₁-C₂₀-alkoxy.

14. The organic solar cell according to claim 13, where A is a group of the formula (VI.1) in which R²³ is phenyl, C₁-C₆-alkyl or hydrogen.

15. The organic solar cell according to any of the preceding claims, in which, in the perylene compound of the formula (I)
R¹ and R⁴ are each phenyl,
R² and R³ together are a radical of the formula (V.1) in which R⁵, R⁶, R⁷ and R⁸ are each hydrogen, and
A is a radical of the formula (VI.1) in which R²³ is phenyl or hydrogen.

16. An organic solar cell, wherein at least one substituted perylene of the formula (I) as defined in any of claims 1 to 15 is used as an electron donor material.

17. The organic solar cell according to any of the preceding claims, wherein the photoactive region comprises at least one substituted perylene of the formula (I) in combination with at least one further, semiconductor material different therefrom.

18. The organic solar cell according to claim 17, wherein the photoactive region comprises, as the further semiconductor material, at least one fullerene and/or fullerene derivative.

19. The organic solar cell according to claim 18, wherein the photoactive region comprises, as the further semiconductor material, C60 or [6,6]-phenyl-C61-butyric acid methyl ester.

20. The organic solar cell according to any of the preceding claims comprising photoactive donor-acceptor transitions in the form of a bulk heterojunction.

21. The organic solar cell according to any of the preceding claims in the form of a single cell or in the form of a tandem cell or in the form of a multijunction cell.

22. The organic solar cell according to any of the preceding claims in the form of a tandem cell, in which one subcell comprises a substituted perylene of the general formula (I) and at least one fullerene or fullerene derivative.

23. The tandem cell according to claim 22, which additionally comprises at least one subcell with an absorption maximum in the relatively long-wave spectral region, preferably in the range from 600 to 900 nm.

24. The use of at least one substituted perylene of the formula (I) as defined in any of claims 1 to 15 as an electron donor material in organic solar cells.

25. A substituted perylene of the formula (I) as defined in any of claims 1 to 15, excluding the compound of the formula (I) in which R¹, R², R³ and R⁴ are all hydrogen, A is a radical of the formula (VI.1) in which R⁵, R⁶, R⁷ and R⁸ are each hydrogen, and R²³ is 2,6-diisopropylphenyl.
